SEQUENCE LISTING (17 sequences)

SEQ ID NO: 1 - DNA, 23 bp, Artificial Sequence
  first constant sequence
acctgatcaccctgtgcgcgtca

SEQ ID NO: 2 - DNA, 21 bp, Artificial Sequence
  second constant sequence
gtacggctcgataagctagca

SEQ ID NO: 3 - DNA, 24 bp, Artificial Sequence
  first constant sequence
agctagccactcggccatgccgcc

SEQ ID NO: 4 - DNA, 24 bp, Artificial Sequence
  second nucleic acid
ggcggcatggccgagtggctagct

SEQ ID NO: 5 - DNA, 16 bp, Artificial Sequence
  third nucleic acid
tgctagcttatcgagc

SEQ ID NO: 6 - DNA, 16 bp, Artificial Sequence
  second nucleic acid
cacagggtgatcaggt

SEQ ID NO: 7 - DNA, 17 bp, Artificial Sequence
  SSP3c
ctgcgcgaactggctag

SEQ ID NO: 8 - DNA, 22 bp, Artificial Sequence
  U2
gatcacactcggccatgccgcc

SEQ ID NO: 9 - DNA, 20 bp, Artificial Sequence
  BclI_U2c
atggccgagtgtgatcaggt

SEQ ID NO: 10 - DNA, 15 bp, Artificial Sequence
  BmtI_SSP3
tgctagccagttcgc

SEQ ID NO: 11 - DNA, 60 bp, Artificial Sequence
  localisation nucleic acid
acctgatcacactcggccatgccgccgcatagacgcatgctctgcgcgaactggctagca

SEQ ID NO: 12 - DNA, 60 bp, Artificial Sequence
  localization nucleic acid
aattccagttcgcgcaggcatagacgcatgctaacgatccgcggctgccgctagcatcga

SEQ ID NO: 13 - DNA, 60 bp, Artificial Sequence
  Localization nucleic acid
ctgtctcttatacacatctgcatagacgcatgctaacgatccgcggctgccgctagcatc

SEQ ID NO: 14 - DNA, 15 bp, Artificial Sequence
  refA
agcatgcgtctatgc

SEQ ID NO: 15 - DNA, 48 bp, Artificial Sequence
  Ran2_BclI_U3
caatcactgaccgccctgaatgatcactagcggcagccgcggatcgtt

SEQ ID NO: 16 - DNA, 16 bp, Artificial Sequence
  BmtI_U3_3ddC
tcgatgctagcggcag

SEQ ID NO: 17 - DNA, 22 bp, Artificial Sequence
  U3
aacgatccgcggctgccgctag

(19) United States
(12) Patent Application Publication
(10) Pub. No.: US 2024/0060122 A1
(43) Pub. Date: Feb. 22, 2024

(54) METHOD FOR SPATIAL MAPPING AND SEQUENCING OF CELLS OR ORGANELLES

(71) Applicants: PARIS SCIENCES ET LETTRES, PARIS (FR); ECOLE SUPERIEURE DE PHYSIQUE ET DE CHIMIE INDUSTRIELLES DE LA VILLE DE PARIS, PARIS (FR); CENTRE NATIONAL DE LA RECHERCHE SCIENTIFIQUE, PARIS (FR)

(72) Inventors: Andrew GRIFFITHS, PARIS (FR); Gaël BLIVET-BAILLY, PARIS (FR); Aurélien DE REYNIÈS, PARIS (FR); Hubert GEISLER, PARIS (FR); Pablo IBANEZ, ARGENTEUIL (FR); Philippe NGHE, SAINT-MANDE (FR)

(21) Appl. No.: 18/260,902

(22) PCT Filed: Jan. 12, 2022
(86) PCT No.: PCT/EP2022/050521

§ 371 Date: Jul. 10, 2023

(30) Foreign Application Priority Data
Jan. 12, 2021 (EP) 21305023.0

(51) Int. Cl. C12Q 1/6841

(52) U.S. Cl. CPC C12Q 1/6841

(57) ABSTRACT

Method for determining tissue positions using an array, which comprise location sequences, the location sequences bind to the tissue samples via ligands. The tissue section which allows localisation tags to be assigned to areas of the tissue comprising a single or plurality of cells or organelles, e.g. about two to ten cells. By triangulation of the localisation tags it is possible to assign a unique relative position in the tissue per cell or organelle.

The present invention concerns a microprinting solution on the surface of a tissue section which allows localisation tags to be assigned to areas of the tissue comprising a single or plurality of cells or organelles, e.g. about two to ten cells.

### TECHNOLOGICAL BACKGROUND

Current sequencing techniques of single cells are very powerful because they allow, for example, access to “omic” information (genomic, epigenomic transcriptomic, proteomic, etc.) for each of the cells in a sample, but they require the dissociation of the cells leading to the loss of localisation information of each of the cells in the starting sample. The invention thus makes it possible for cells of a tissue to be dissociated, analysed and sequenced individually (single cell) using current techniques (droplet microfluidics, valve microfluidics, microplates, thermoactuable hydrogels), with the possibility of combining the localisation tag and the identity tag of each cell in order to determine their position in the starting sample.

Indeed, a major issue e.g., in oncology is how to accurately characterize the tumour/microenvironment interactions, which requires access to both molecular signals and the intra-tissue spatial localisation of the cells that exchange these signals. These interactions may play a major role in the survival or death of tumour cells. A better understanding of these interactions would help to overcome the adaptive mechanisms taking place in the tumour system.

Although “omics” technologies have revolutionized molecular biology by making genomic, transcriptomic, epigenomic and proteomic analyses possible on a very large scale, they have until recently made it possible to obtain only “average” profiles of multiple cells that do not take into account the cellular heterogeneity present in healthy and pathological tissues. Recently, however, a number of technologies for single cell analysis have been developed. In particular, droplet microfluidic systems in which individual cells are co-encapsulated in droplets with beads harbouring barcoded primers allow the sequencing of RNA at the single-cell level (scRNA-seq) from several thousands of cells.

However, in these systems, the cells in the tissue are dissociated prior to analysis and there is no correlation between the single cell sequencing data and the localisation of these same cells in the original tissue.

Single-cell analysis technologies allowing spatial localisation of the measured signals are currently very limited, both in terms of number of targets (immunohistochemistry, in situ RNA hybridization) and number of cells (e.g. sequencing of individual cells after laser capture microdissection (LCM)). Although “spatial transcriptomics” (Vickovic, Sanja, et al. “High-definition spatial transcriptomics for in situ tissue profiling.” Nature methods 16.10 (2019): 987-990.) and “Slide-seq” (Rodrigues, Samuel G., et al. “Slide-seq: A scalable technology for measuring genome-wide expression at high spatial resolution.” Science 363.6434 (2019): 1463-1467) systems allow spatial resolution in individual tissue sections, they do not provide access to single cell data. Fluorescence in situ sequencing (FISSEQ) uses in situ sequencing to spatially localise the expression of multiple genes in fixed tissues, with a short reading (30 bases) and with only about 200 mRNA readings per cell (compared to about 40,000 in scRNA-seq). Digital Spatial Profiler (DSP) is a platform developed by Nanostring based on the sequencing of photocleavable oligonucleotide markers released from a targeted tissue area by UV exposure. Data on the localisation of cells in the tissue provide a numerical and spatial profile of RNA or target abundance. However, this technique does not provide access to the complete transcriptome, does not have unicellular resolution, and allows analysis of only a small number of areas.

In summary, the current technological tools to study cellular interactions in tissues interaction at the single-cell level are still limited because they only allow spatial localisation of signals from a limited number of molecular targets for a large number of cells, or measurement of a large number of molecular targets for a limited number of localised (microdissected) cells, or measurement of a large number of molecular targets on thousands of non-localised cells.

Currently, the coupling, for thousands of individual cells, of both (i) intra-tissue spatial localisation information of each cell to (ii) the measurement of molecular signals from each of these same cells is clearly identified as a major technological challenge and as a clinical need in order to better understand, for example, the adaptation mechanisms occurring in tumours and to adjust patient treatment accordingly.

The methods and kits provided by the invention fulfil this need.

### SUMMARY OF THE INVENTION

The invention relates to a method for labelling individual cells or organelles within a tissue sample with a localisation nucleic acid, the method comprising:
a) providing a nucleic acid array comprising a substrate and localisation nucleic acids, wherein each localisation nucleic acid comprises, from the 5′ end to the 3′ end:
   i. a first constant sequence;
   ii. a localisation sequence that is indicative of the position of the nucleic acid on the nucleic acid array; and
   iii. a second constant sequence;
wherein the localisation nucleic acid is attached, covalently or non-covalently, to the substrate of the nucleic acid array;
b) contacting the nucleic acid array with a tissue sample so that the localisation nucleic acids are at the interface between the nucleic acid array and the tissue sample:
c) releasing all or part of the localisation nucleic acids comprising all or part of the first constant sequence, the localisation sequence, and all or part of the second constant sequence or a nucleic acid complementary thereto;
d) binding of the released nucleic acids comprising the localisation sequence or its complementary sequence, to cells or organelles of the tissue sample via a ligand bound to a receptor or receptors of cells or organelles in the tissue sample;
e) dissociating the tissue sample and recovering individualized cells or organelles, wherein at least a sub-population of the individualized cells or organelles is labelled with the nucleic acid comprising the localisation sequence or its complementary sequence.

The invention also relates to a method for labelling individual cells or organelles within a tissue sample with a localisation nucleic acid, the method comprising:
a) providing a nucleic acid array comprising a substrate and localisation nucleic acids, wherein each localisation nucleic acid comprises, from the 5′ end to the 3′ end:
   i. a first constant sequence;
   ii. a localisation sequence that is indicative of the position of the nucleic acid on the nucleic acid array; and
   iii. a second constant sequence;
wherein the localisation nucleic acid is attached, covalently or non-covalently, to the substrate of the nucleic acid array;
b) contacting the nucleic acid array with a polymer stamp so that the localisation nucleic acids are at the interface between the nucleic acid array and the polymer stamp;
c) releasing all or part of the localisation nucleic acids comprising all or part of the first constant sequence, the localisation sequence, and all or part of the second constant sequence or a nucleic acid complementary thereto;
d) contacting the polymer stamp with a tissue sample:
e) binding of the released nucleic acids comprising the localisation sequence or its complementary sequence, to cells or organelles of the tissue sample via a ligand bound to a receptor or receptors of cells or organelles in the tissue sample;
f) dissociating the tissue sample and recovering individualized cells or organelles, wherein at least a sub-population of the individualised cells or organelles is labelled with the nucleic acid comprising the localisation sequence or its complementary sequence.

The invention also relates to a method for labelling individual cells or organelles within a tissue sample with a localisation nucleic acid, the method comprising:
A) providing a nucleic acid array comprising a substrate and localisation nucleic acids, wherein each localisation nucleic acid comprises, from the 5′ end to the 3′ end:
   i. a first (5′) constant sequence;
   ii. a localisation sequence that is indicative of the position of the nucleic acid on the nucleic acid array; and
   iii. a second (3′) constant sequence;
wherein the localisation nucleic acid is attached, covalently or non-covalently, to the substrate of the nucleic acid array;
B) providing a tissue sample labelled with a ligand which binds to a receptor or receptors of cells or organelles in the tissue sample, wherein the ligand is attached, covalently or non-covalently, to a capture nucleic acid which comprises at its 3′ end a sequence complementary to all or part of said second (3′) constant sequence;
C) contacting the nucleic acid array with the tissue sample so that the localisation nucleic acids are at the interface between the nucleic acid array and the tissue sample;
D) hybridising the localisation nucleic acid and capture nucleic acid, and extending the 3′ end of capture nucleic acid by polymerisation, thereby synthesising a sequence complementary to said localisation nucleic acid;
E) releasing all or part of the localisation nucleic acids comprising all or part of the first (5′) constant sequence, the localisation sequence, and all or part of the second (3′) constant sequence;
F) dissociating the tissue sample and recovering individualized cells or organelles, wherein at least a sub-population of the individualised cells or organelles is labelled with the capture nucleic acid comprising a sequence complementary to the localisation sequence.

The invention also provides for a kit for labelling individual cells or organelles within a tissue sample with a nucleic acid localisation sequence, which comprises:
a) A nucleic acid array comprising localisation nucleic acids, wherein each localisation nucleic acid comprises, from the 5′ end to the 3′ end:
   a first constant sequence;
   a localisation sequence that is indicative of the position of the nucleic acid on the nucleic acid array; and
   a second constant sequence;
wherein the localisation nucleic acid is attached, covalently or non-covalently, to the substrate of the nucleic acid array;
b) at least one type of ligand (for example an antibody) specific for a receptor of cells or organelles in the tissue, preferably a receptor ubiquitously present at the surface of the cells of a tissue sample, wherein the at least one type of ligand is optionally bound to a member of a non-covalent interacting pair (for example streptavidin/biotin); and

c) optionally both members of the non-covalent interacting pair.

The invention further relates to a method of mapping and sequencing individual cells or organelles of a tissue sample, the method comprising:
a) providing individualized cells or organelles labelled with a nucleic acid comprising a localisation sequence as obtainable or obtained by the method of labelling of the invention;
b) trapping the individualized cells or organelles labelled with the nucleic acid comprising the localisation sequence in a compartment, wherein the compartment comprises a compartment-specific nucleic acid comprising a compartment-specific sequence and one or any combination of the following sequences for nucleic acid labeling and further sequencing: hybridization site, ligation site or recombination site;
c) Optionally analysing captured cells, organelles and/or molecules they secrete, using optical detection;
d) Optionally, lysing trapped cells, or cells and organelles, thereby releasing nucleic acids from the cells or organelles in the compartments;
e) Associating the compartment-specific sequence with the nucleic acids released from the cell or organelle in the compartments and the nucleic acids comprising the localisation sequence;
f) Recovering the nucleic acids produced at step e) from the compartments and sequencing recovered nucleic acids; and
g) Defining sequenced nucleic acid comprising the same compartment-specific sequence as originating from the same single cell, and mapping the position of the single cell originally on the tissue sample based on the localisation sequence(s) contained in part of the nucleic acid that comprises said same compartment-specific sequence, thereby combining mapping and sequencing information of the individual cells of the tissue sample.

The invention also relates to a kit for mapping and sequencing individual cells or organelles of a tissue sample which comprises, as constituents of the kit:
a) the constituents of the kit for labelling individual cells or organelles within a tissue sample;
b) a compartment as defined in the method of mapping and sequencing individual cells or organelles.

### DETAILED DESCRIPTION OF THE INVENTION

The inventors have designed a method allowing cells from a specific region of a tissue section to be labelled with ligands themselves labelled with localisation nucleic acids, comprising a localisation sequence that is indicative of position, using microcontact printing prior to tissue dissociation and single-cell sequencing to map the information from sequencing onto the position of each cell in the tissue section of origin (see exemplary embodiment of the method in FIG. 1).

A protocol for labelling tissue sections has been developed using nucleic acid arrays, for instance commercial DNA arrays. These nucleic acid arrays consist of a large number (up to 1,000,000 spots) of small (down to 30 μm diameter), dense spots comprised of short nucleic acids (up to 10⁹ nucleic acids per spot, each nucleic acid being up to 60-80 nucleotides long), anchored to the substrate at their 3′ end (and with a free 5′ end), or anchored at their 5′ end (and with a free 3′ end). According to the invention, the nucleic acid array comprises localisation nucleic acids, each of which carries a barcode with a sequence specific to each spot (localisation sequence') flanked by two constant regions that are used in the method to release the localisation nucleic acids from the array and to bind to the cells (or organelles thereof) of the tissue through a ligand having specificity for a receptor or receptors of cells in the tissue sample (see principle of the method in FIG. 1).

It is thus provided a method for labelling individual cells or organelles within a tissue sample with a localisation nucleic acid, the method comprising:
a) providing a nucleic acid array comprising a substrate and localisation nucleic acids, wherein each localisation nucleic acid comprises, from the 5′ end to the 3′ end:
   i. a first (5′) constant sequence;
   ii. a localisation sequence that is indicative of the position of the nucleic acid on the nucleic acid array; and
   iii. a second (3′) constant sequence;
wherein the localisation nucleic acid is attached, covalently or non-covalently, to the substrate of the nucleic acid array;
b) contacting the nucleic acid array with a tissue sample so that the localisation nucleic acids are at the interface between the nucleic acid array and the tissue sample:
c) releasing all or part of the localisation nucleic acids comprising all or part of the first constant sequence, the localisation sequence, and all or part of the second constant sequence, or nucleic acids complementary thereto;
d) binding of the released nucleic acids comprising the localisation sequence or its complementary sequence, to cells of the tissue sample via a ligand bound to a receptor or receptors of cells in the tissue sample; and
e) dissociating the tissue sample and recovering individualized cells or organelles, wherein at least a sub-population, preferably substantially all, of the individualized cells or organelles is labelled with the nucleic acid comprising the localisation sequence or its complementary sequence.

Depending on the 5′ or 3′ anchoring of the localisation nucleic acids to the substrate of the array, step c) comprises releasing all or part of the localisation nucleic acids comprising (1) all or part of the first (5′) constant sequence, and the localisation sequence, and the second (3′) constant sequence (5′ anchoring), or (2) the first (5′) constant sequence, the localisation sequence, and all or part of the second (3′) constant sequence (3′ anchoring).

In certain embodiments of the method, prior to step b), the tissue sample is incubated with the ligand that binds to a receptor or receptors of cells, in particular to the surface or inside all cells, or a sub-set of cells, in the tissue sample, and which is bound possibly on organelles.

Step c) of the method may further comprise depositing an aqueous solution comprising soluble monomers and/or polymers at the interface between the nucleic acid array and the tissue sample, at the interface of the nucleic acid array and the labelled tissue sample, and preferably at the surface of the tissue sample, before contacting the nucleic acid array and the tissue sample. The monomers and/or polymers may be reticulated or not. This aqueous solution serves to reduce the rate of diffusion of the released localisation nucleic acids, for example by increasing viscosity, and to maximise the surface of contact between the nucleic acid array and the tissue sample.

Where the method involves the use of an endonuclease for releasing all or part of the localisation nucleic acids, step a) of the method preferably further comprises, depositing an aqueous solution comprising soluble monomers and/or polymers comprising said endonuclease at the surface of the nucleic acid array and optionally lyophilizing the solution/endonuclease layer, before contacting the nucleic acid array and the tissue sample at step b). Depending on the embodiment of the method, said endonuclease may be the so-called “first endonuclease” or “second endonuclease” as defined afterwards.

Where the method involves the use of nucleic acids complementary to the nucleic acid array for modifying the nucleic acids array before contacting the nucleic acid array and the tissue sample at step b), step a) of the method preferably further comprises, depositing a solution comprising nucleic acids, polymerase, ligase, and/or exonuclease at the surface of the nucleic acid array.

Where the method involves the use of a polymerase and/or exonuclease, or any other enzyme, step a) of the method may further comprises, depositing an aqueous solution comprising soluble monomers and/or polymers comprising polymerase and/or exonuclease at the surface of the nucleic acid array and lyophilizing the solution/endonuclease layer, before contacting the nucleic acid array and the tissue sample at step b).

In a particular embodiment of the method:
at step a), the provided nucleic acid array further comprises a layer comprising an endonuclease used for releasing all or part of the localisation nucleic acids (a so-called “first endonuclease”, or “second endonuclease” as defined afterwards), a polymerase and/or exonuclease, or any other enzyme, deposited and lyophilised onto the surface of the nucleic acid array harbouring the nucleic acids, and the nucleic array is in lyophilized form; and
step c) further comprises depositing a layer of an aqueous solution soluble comprising monomers and/or polymers at the interface of the nucleic acid array and the tissue sample, preferably on top of the nucleic acid array, before contacting the nucleic acid array and the tissue sample.

Step c) of contacting is preferably performed at 0-40° C., preferably 25-37° C., still preferably 37° C.

Between step d) of binding and step e) of dissociating, a washing step may be implemented to increase the signal/noise ratio by limiting diffusion of released localisation nucleic acids.

In an exemplary embodiment of the method, the 3′-end of the localisation nucleic acids is attached to the substrate of the nucleic acid array, and the sequence of the 5′-constant region is used to hybridize a second oligonucleotide with a biotin moiety at its 3′ end which allows the localisation nucleic acids to be coupled to ligands used to label cells or organelles in tissue sections, by virtue of binding of the biotin moiety to avidin or streptavidin, which is in turn coupled to the ligand(s), either via covalently or via non-covalent binding to ligand(s). It has been shown that the barcoded (localisation) nucleic acid can be effectively released from the nucleic acid array by hybridizing a third oligonucleotide into the 3′-constant regions, thereby creating a target restriction site (e.g. for Bmtl) and cleaving with said restriction endonuclease (FIG. 2). The released barcoded nucleic acids diffuse in the space between the nucleic array and the tissue and bind to cells or cell organelles of the tissue sample, thereby labelling the cells or organelles with localisation sequences and mapping cells or organelles on the tissue. The tissue sample is then dissociated and the dissociated cells or organelles can be analysed by single-cell or single organelle sequencing: by simultaneously sequencing the localisation sequence and nucleic acids released from the cells or organelles, and/or nucleic acid tags attached to ligands, such as antibodies, bound to cellular markers, thereby making it possible to map the single-cell or organelle “omics” information (genomic, epigenomic, transcriptomic, proteomic etc.) to the original position of each cell or organelle in the tissue sample before tissue dissociation.

In the method for labelling individual cells or organelles within a tissue sample, the localisation nucleic acid is
i) either attached (at step a), covalently or non-covalently, to the ligand which binds to the receptor or receptors of cells,
ii) or becomes attached (after release at step c) to the ligand which binds to the receptor or receptors of cells, at step d), indirectly via a non-covalent interacting pair (such as biotin-avidin or biotin-streptavidin), or via a capture nucleic acid attached, covalently or non-covalently, to the ligand.

Four modes of implementation of the method are therefore encompassed:

### I—Method for Labelling Individual Cells of a Tissue Sample: Localisation Nucleic Acid Attached, Covalently or Non-Covalently, to the Ligand before Contacting with Tissue

It is provided a method for labelling individual cells or organelles within a tissue sample with a nucleic acid localization sequence, the method comprising:
a) providing a nucleic acid array comprising a substrate and localisation nucleic acids, wherein each localisation nucleic acid comprises, from the 5′ end to the 3′ end:
   i. a first (5′) constant sequence;
   ii. a localisation sequence that is indicative of the position of the nucleic acid on the nucleic acid array; and
   iii. a second (3′) constant sequence;
wherein the localisation nucleic acid is attached, covalently or non-covalently, to the substrate of the nucleic acid array;
wherein the localisation nucleic acid is attached, covalently or non-covalently to the ligand which binds to a receptor or receptors of cells or organelles in the tissue sample;
b) contacting the nucleic acid array with the tissue sample so that the localisation nucleic acids are at the interface between the nucleic acid array and the tissue sample:
c) releasing all or part of the localisation nucleic acids comprising all or part of the first constant sequence, and the localisation sequence and second constant sequence, and the ligand, covalently or non-covalently, attached to the released localisation nucleic acids;
d) binding of the released nucleic acids comprising the localisation sequence to cells of the tissue sample via the ligand;
e) dissociating the tissue sample and recovering individualized cells, wherein at least a sub-population, preferably substantially all, of the individualized cells is labelled with a nucleic acid comprising the localisation sequence

### II—Method for Labelling Individual Cells of a Tissue Sample: Localisation Nucleic Acid Attached, Covalently or Non-Covalently, to a Member of a Non-Covalent Interacting Pair before Contacting with Tissue

It is provided a method for labelling individual cells or organelles within a tissue, the method comprising:
a) providing a nucleic acid array comprising a substrate and localisation nucleic acids, wherein each localisation nucleic acid comprises, from the 5′ end to the 3′ end:
   i. a first (5′) constant sequence;
   ii. a localisation sequence that is indicative of the position of the nucleic acid on the nucleic acid array; and
   iii. a second (3′) constant sequence;
wherein the localisation nucleic acid is attached, covalently or non-covalently, to the substrate of the nucleic acid array;
wherein the localisation nucleic acid is attached, covalently or non-covalently to a first member of a non-covalent interacting pair;
b) providing a tissue sample labelled with a ligand which binds to a receptor or receptors of cells or organelles in the tissue sample, wherein the ligand is attached to the second member of the non-covalent interacting pair;
c) contacting the nucleic acid array with the tissue sample so that the localisation nucleic acids are at the interface between the nucleic acid array and the tissue sample:
d) releasing all or part of the localisation nucleic acids comprising all or part of the first constant sequence, and the localisation sequence and second constant sequence and the first member of the non-covalent interacting pair, covalently or non-covalently, bound to the released localisation nucleic acids;
e) binding of the released nucleic acids comprising the localisation sequence to the ligands bound a receptor or receptors of cells of the tissue sample, via the non-covalent binding of the first and second members of the non-covalent interacting pair;
f) dissociating the tissue sample and recovering individualized cells, wherein at least a sub-population, preferably substantially all, of the individualized cells is labelled with a nucleic acid comprising the localisation sequence.

### III—Method for Labelling Individual Cells of a Tissue Sample: Capture of the Released Localisation Nucleic Acid by a Capture Nucleic Acid Attached, Covalently or Non-Covalently, to the Ligand

It is provided a method for labelling individual cells or organelles within a tissue, the method comprising:
a) providing a nucleic acid array comprising a substrate and localisation nucleic acids, wherein each localisation nucleic acid comprises, from the 5′ end to the 3′ end:
   i. a first (5′) constant sequence;
   ii. a localisation sequence that is indicative of the position of the nucleic acid on the nucleic acid array; and
   iii. a second (3′) constant sequence;
wherein the localisation nucleic acid is attached, covalently or non-covalently, to the substrate of the nucleic acid array;
b) providing a tissue sample labelled with a ligand which binds to a receptor or receptors of cells or organelles in the tissue sample, wherein the ligand is attached, covalently or non-covalently, to a capture nucleic acid;
c) contacting the nucleic acid array with the tissue sample so that the localisation nucleic acids are at the interface between the nucleic acid array and the tissue sample;
d) releasing all or part of the localisation nucleic acids comprising all or part of the first constant sequence, the localisation sequence, and the second constant sequence, or comprising all or part of the localisation nucleic acids comprising the first constant sequence, the localisation sequence, and all or part of the second constant sequence, or releasing a nucleic acid complementary thereto;
e) binding of the released nucleic acids comprising the localisation sequence or its complementary sequence to the ligands bound to a receptor or receptors of cells of the tissue sample, via the capture nucleic acid;
f) dissociating the tissue sample and recovering individualized cells, wherein at least a sub-population, preferably substantially all, of the individualized cells is labelled with a nucleic acid comprising the localisation sequence or its complementary sequence.

The capture nucleic acid captures the released part or entire localisation nucleic acid, or its complementary nucleic acid, by hybridisation or ligation.

### IV—Method for Labelling Individual Cells of a Tissue Sample: Capture of the Stamp-Released Localisation Nucleic Acid by a Capture Nucleic Acid Attached, Covalently or Non-Covalently, to the Ligand

It is provided a method for labelling individual cells or organelles within a tissue sample with a localisation nucleic acid, the method comprising:
a) providing a nucleic acid array comprising a substrate and localisation nucleic acids, wherein each localisation nucleic acid comprises, from the 5′ end to the 3′ end:
   i. a first constant sequence;
   ii. a localisation sequence that is indicative of the position of the nucleic acid on the nucleic acid array; and
   iii. a second constant sequence;
wherein the localisation nucleic acid is attached, covalently or non-covalently, to the substrate of the nucleic acid array;
b) contacting the nucleic acid array with a polymer stamp so that the localisation nucleic acids are at the interface between the nucleic acid array and the polymer stamp;
c) releasing all or part of the localisation nucleic acids comprising all or part of the first constant sequence, the localisation sequence, and all or part of the second constant sequence or a nucleic acid complementary thereto;
d) contacting the polymer stamp with a tissue sample:
e) binding of the released nucleic acids comprising the localisation sequence or its complementary sequence, to cells or organelles of the tissue sample via a ligand bound to a receptor or receptors of cells or organelles in the tissue sample;
f) dissociating the tissue sample and recovering individualized cells or organelles, wherein at least a sub-population of the individualised cells or organelles is labelled with the nucleic acid comprising the localisation sequence or its complementary sequence.

The capture nucleic acid captures the released part or entire localisation nucleic acid, or its complementary nucleic acid, by hybridisation or ligation.

Non-covalent binding of the localisation nucleic acid, or of the capture nucleic acid, to the ligand, can typically be implemented using a non-covalent interacting pair, such as biotin-avidin or biotin-streptavidin. In an embodiment a first member of the non-covalent interacting pair, e.g. avidin or streptavidin, is covalently bound to the ligand, and the localisation nucleic acid is covalently or non-covalently (e.g. by hybridizing to a biotin labelled complementary nucleic acid) attached to the other member of the non-covalent interacting pair, e.g. biotin. In another embodiment, a first member of the non-covalent interacting pair, e.g. avidin or streptavidin, is covalently or non-covalently bound to the ligand, and the capture nucleic acid is attached to the other member of the non-covalent interacting pair, e.g. biotin.

The localisation nucleic acid is attached to the substrate of the nucleic acid array either on its 5′ side or on its 3′ side. Depending on the orientation (5′-3′ or 3′-5′) of the localisation nucleic acids attached to the substrate, and of the mode of biding of the release localisation nucleic acid to the receptor(s) of cells or cell organelles, different modes of implementation of the method for labelling individual cells within a tissue sample are available.

According to an embodiment, all nucleic acids used in the method or kit are DNAs.

### Polymer Stamp

The polymer stamp consists of a cross-linked polymer matrix carrying a relief pattern that allows the transfer of localisation nucleic acids from the substrate to the tissue via microcontact printing (D. Qin, Y. Xia, and G. M. Whitesides. 2010. “Soft Lithography for Micro- and Nanoscale Patterning.” Nature Protocol, 5, Pp. 491-502.). The stamp may, for example be manufactured in poly(dimethyl siloxane) using soft lithography. The stamp may also, for example, form a highly hydrophilic network (for example, a polymer that can contain over 80% water). Prior contacting the nucleic acid array, the polymer stamp may be wetted with an aqueous solution comprising a restriction enzyme, a nicking enzyme, or a polymerase, or combinations thereof, allowing release of all or part of the localisation nucleic acids comprising all or part of the first constant sequence, the localisation sequence, and all or part of the second constant sequence or a nucleic acid complementary thereto.

### Tissue Sample

Preferably, the tissue sample comprises a planar surface, and is for instance a tissue section. The tissue can be either fresh, frozen or fixed.

According to an embodiment, the tissue sample is a tumour tissue sample.

Dissociation of tissue sample can be achieved using for instance collagenase I, Dnase I and hyaluronidase.

### Nucleic Acid Array

The nucleic acid array is typically an RNA or DNA array, which comprises a large number (up to 1,000,000 spots) of small (down to 30 μm diameter), dense spots comprising (up to 10⁹ nucleic acids per spot) nucleic acids anchored to the substrate at their 3′ or 5′ end. Preferably, the nucleic acid array is a DNA array.

According to an embodiment, the localisation nucleic acids form spots at the surface of the nucleic acid array, and all localisation nucleic acids of a same spot comprise a same localisation sequence which is specific for one or more spots. According to another embodiment, the localisation nucleic acids form spots at the surface of the nucleic acid array, and localisation nucleic acids of a same spot comprise altogether more than one localisation sequence which is specific for one or more spots.

In some instances, multiple spots or all of the spots of the nucleic acid array comprise a nucleic acid fluorescently labelled to provide visual reference points that can be used to map the bio-informatically reconstructed map of the cells to a fluorescent picture of the original tissue. The fluorescently labelled nucleic acids may be either attached to the nucleic acid array, or hybridised to unlabelled nucleic acids attached to the nucleic acid array. A single fluorescently labelled nucleic acid may be spatially repeated in different spots of the nucleic acid array, or alternatively different fluorescently labelled nucleic acids are attached to the spots of the nucleic acid array.

### Localisation Nucleic Acids

The localisation nucleic acid may be RNA and/or DNA, and preferably consists of a DNA sequence.

In the localisation nucleic acids, the localisation sequence is flanked by the first constant sequence, at its 5′ end, and by the second constant sequence, at its 3′ end.

The stretch consisting of the first constant sequence, localisation sequence, and second constant sequence is preferably at most 80, 70 or preferably 60 nucleotide long (e.g. when nucleic acids are synthesised on the nucleic acid array), but longer sequences can also be used (e.g. when nucleic acids are synthesised off the nucleic acid array and then spotted). Typically, each of the first constant sequence, localisation sequence, and second constant sequence is 15 to 30 nucleotides long, preferably 18 to 25 nucleotide long.

Preferably, the sequence of the first constant sequence is identical in all localisation nucleic acids. Preferably, the sequence of the second constant sequence is identical in all localisation nucleic acids. The sequences of the first constant sequence and second constant sequences differ from each other.

For example, the first constant sequence (at the 5′ end of the localisation sequence) comprises or consists of ACCTGATCACCCTGTGCGCGTCA (SEQ ID NO: 1).

For example, the second constant sequence (at the 3′ end of the localisation sequence) comprises or consists of GTACGGCTCGATAAGCTAGCA (SEQ ID NO: 2).

Localisation nucleic acids generally form spots at the surface of the substrate of the nucleic acid array. According to an embodiment, all localisation nucleic acids of a same spot on the substrate of the nucleic acid array comprise the same localisation sequence which is specific for one or more spots. In an embodiment, the localisation sequence is specific for a single spot. In another embodiment, the localisation sequence is specific for multiple spots (e.g. 2, 3, 4, 5 or more spots), preferably multiple contiguous spots (e.g. 2, 3, 4 or 5 or more contiguous spots). The different localisation sequences can be designed randomly or by a method which guarantees a minimum distance in sequence space between two localisation sequences.

The localisation nucleic acids may further comprise a spacer sequence at its 5′ or 3′ end. Examples of spacer sequences that can be used in the frame of the invention typically include polydT, PEG, carbon chain C6 or C12 amino spacer.

According to an embodiment, the 5′ end of the localisation nucleic acid is attached, covalently or non-covalently, optionally via a spacer sequence and/or linker, to the substrate of the nucleic acid array. In an embodiment, the 5′ end of the localisation nucleic acid is attached, covalently or non-covalently, via a spacer sequence and/or linker to the substrate of the nucleic acid array.

According to an embodiment, the 3′ end of the localisation nucleic acid is attached, covalently or non-covalently, optionally via a spacer sequence and/or linker, to the substrate of the nucleic acid array. In an embodiment, the 3′ end of the localisation nucleic acid is attached, covalently or non-covalently, via a spacer sequence and/or linker to the substrate of the nucleic acid array.

The spacer sequence is preferably at most 20 nucleotide long.

Examples of linkers that can be used in the frame of the invention typically include polydT, PEG, carbon chain C6 or C12 amino spacer.

### Ligand

The ligand binds to a receptor or to receptors of cells in the tissue sample.

According to an embodiment, the ligand binds to a receptor or receptors at the surface or inside the cells. According to another embodiment, the ligand binds to a receptor or receptors at the surface of or inside an organelle of the cells. As used herein an organelle includes, without limitation, mitochondria, chloroplast, endoplasmic reticulum, flagellum, Golgi apparatus, nucleus, and vacuole.

For instance, the receptor or receptors present at the surface or inside the cells or organelles of the tissue sample is selected from the group consisting of a cell or organelles surface protein (e.g. CD45, CD3, CD19, CD98, CD298, β2 microglobulin), a carbohydrate (e.g. Mannose, Galactose, N-acetylglucosamine), and the lipid bilayer of cells or organelles.

Preferably the ligand is selected from the group consisting of an antibody, an aptamer, a lectin, and a peptide.

According to an embodiment, the receptor or receptors at the surface or inside the cells or any organelles of the cells bound by the ligand is ubiquitously present at the surface or inside all or most of the cells or organelles of the cells in the tissue sample (e.g. CD98, CD298, β2 microglobulin, Mannose, Galactose).

According to another embodiment, the receptor or receptors at the surface of cells bound by the ligand is present inside or at the surface of only a sub-set of the cells or organelles in the tissue sample (e.g. CD45, CD3, CD19).

### 5′ Anchoring

The 5′ end of the localisation nucleic acid is attached, covalently or non-covalently, optionally via a spacer sequence and/or linker, to the substrate of the nucleic acid array. In an embodiment, the 5′ end of the localisation nucleic acid is attached, covalently or non-covalently, via a spacer sequence and/or linker to the substrate of the nucleic acid array.

In an embodiment of the method, the spacer sequence, linker or 5′ end of the localisation nucleic acid comprises a photo-cleavable or chemically-cleavable link and in step c) of the method, all or part of the localisation nucleic acid comprising i) all or part of the first constant sequence, ii) the localisation sequence, and iii) the second constant sequence is released from the nucleic acid array by photo- or chemically-induced cleavage, as appropriate, of said photo-cleavable or chemically-cleavable link.

In another embodiment of the method, step a) preferably comprises hybridizing a “second nucleic acid” by complementarity to all or part of the first constant sequence (5′ constant sequence). For the purpose of differentiating the different nucleic acids implemented in the method, said nucleic acid having complementarity with, or complementary to, all or part of the first constant sequence is called “second nucleic acid”. The second nucleic acid is preferably a DNA. Preferably, the hybridized second nucleic acid forms, together with the first constant sequence, a stretch of double stranded DNA of at least 10 base pairs. Preferably, the hybridized second nucleic acid forms, together with the first constant sequence, a stretch of double stranded DNA containing a first restriction site for a first endonuclease (see e.g. FIGS. 12-13, 15-16, 17-18, 20-22). The endonuclease may generate sticky ends, blunt ends, or hydrolyse only the strand of first constant sequence in the double stranded DNA (generating nicked DNA).

For example, where the first constant sequence comprises or consists of AGCTAGC CACTCGGCCATGCCGCC (SEQ ID NO: 3), the second nucleic acid may comprise or consist of sequence GGCGGCATGGCCGAGTGGCTAGCT (SEQ ID NO: 4). Said pair of first constant sequence and second nucleic acid, when hybridised, form a stretch of double stranded DNA containing a restriction site for Nhel.

The first endonuclease is used in the method to release all or part of the localisation nucleic acid from the substrate of the nucleic acid array. Accordingly, in step c) in method I or step d) in methods II and III, all or part of the localisation nucleic acid comprising i) all or part of the first (5′) constant sequence, ii) the localisation sequence, and iii) the second (3′) constant sequence is released from the nucleic acid array by cleavage catalysed by the first endonuclease. In embodiments wherein the hybridised second nucleic acid is attached at its 3′ end to a first member of a non-covalent interacting pair, or to the ligand, the released localisation nucleic acid remains hybridised to the second nucleic acid bound to the first member of a non-covalent interacting pair or to the ligand.

According to a specific aspect of the method, the second nucleic acid is covalently bound at its 3′ end to the ligand which binds to the receptor or receptors of cells or organelles (see e.g. FIG. 11). Alternatively, the second nucleic acid is covalently bound at its 3′ end to a first member of a non-covalent interacting pair, such a first member of the biotin-streptavidin pair, preferably biotin, while the ligand which binds to the receptor(s) of cells or organelles is attached to the other member of the non-covalent interacting pair, such a second member of the biotin-streptavidin pair, preferably streptavidin (see e.g. FIG. 12). According to these aspects of the method, the hybridized second nucleic acid forms, together with the first (5′) constant sequence, a stretch of double stranded DNA containing a first restriction site, which is a nicking site for a nicking endonuclease when the second nucleic acid is covalently bound at its 3′ end to a first member of a non-covalent interacting pair or to the ligand. When a nicking endonuclease is involved, it is used in the method to release, from the substrate of the nucleic acid array, a part of the localisation nucleic acid hybridised to the second nucleic acid bound to the ligand or member of the non-covalent interacting pair.

According to another specific aspect of the method, a ligand is used which is covalently or non-covalently attached to a capture nucleic acid comprising a region fully or partly complementary to the second (3′) constant sequence (see e.g. FIG. 13). In step d), the released localisation nucleic acid comprises i) all or part of the first (5′) constant sequence, ii) the localisation sequence, and iii) the second (3′) constant sequence hybridised to the complementary region of the capture nucleic acid. Step d) further includes (1) extending the 3′ end of the second (3′) constant sequence and the 3′ end of the complementary region of the capture nucleic acid, using a template-dependent DNA polymerase (e.g. phi29), thereby forming an extended double-stranded nucleic acid, and (2) removing 5′ mononucleotides from the extended double-stranded nucleic acid, using an exonuclease acting in the 5′ to 3′ direction (e.g. T7 exonuclease).

According to another specific aspect of the method, a ligand is used which is covalently or non-covalently attached to a capture nucleic acid comprising a strand complementary to part of the first (5′) constant sequence (see e.g. FIG. 15). In step d) the released localisation nucleic acids comprising i) all or part of the first (5′) constant sequence, ii) the localisation sequence, and iii) the second (3′) constant sequence hybridise to the complementary region of the capture nucleic acid. Step d) further includes (1) using a template-dependant non-strand displacing DNA polymerase (e.g. Phusion) to synthesise a strand complementary to the capture nucleic acids and (2) ligating the localisation nucleic acids with the capture nucleic acids.

In an embodiment (see e.g. FIG. 16), the first endonuclease is used in step c) to release all or part of the localisation nucleic acids from the substrate of the nucleic acid array, wherein released localisation nucleic acid comprises i) all or part of the first (5′) constant sequence, ii) the localisation sequence, and iii) the second (3′) constant sequence. For binding the released localisation nucleic acids to cells of the tissue sample via the ligand bound to a receptor or receptors of cells, a ligand is used which is covalently or non-covalently attached to a partly double stranded capture nucleic acid, wherein the protruding strand is complementary to 3′ end of the second constant sequence. Step d) further includes repairing with a ligase the nick between partly double stranded capture nucleic acid and the partly doubled stranded nucleic acid formed by the protruding strand of the capture nucleic acid hybridised to the second constant sequence of the released localisation nucleic acids.

According to a variant of the above embodiments, a photo- or chemically-cleavable link is used instead of a stretch of double stranded DNA containing a restriction site for an endonuclease .

In an embodiment, the capture nucleic acid forms, together with the second (3′) constant sequence or first (5′) constant sequence, a stretch of double stranded DNA containing a second restriction site for a second endonuclease. This second restriction site for a second endonuclease makes it possible to release from the labelled cells, or organelles, the nucleic acids comprising the localisation sequences or its complementary sequence that are bound thereto, by cleavage with said second endonuclease. This release may be implemented in a subsequent method of analysing the labelled individualised cells or organelles (see following section “Method of mapping and sequencing individual cells or organelles of a tissue sample”).

In the embodiment of the method involving 5′ anchoring of the localisation nucleic acid, step a) may alternatively or also comprise hybridizing a third nucleic acid by complementarity to all or part of the second constant sequence (3′ constant sequence). For the purpose of differentiating the different nucleic acids implemented in the method, said nucleic acid having complementarity with, or complementary to, all or part of the second constant sequence is called “third nucleic acid”, although there might be no “second nucleic acid” implemented in the method. The third nucleic acid is preferably a DNA.

The third nucleic acid does not hybridise to the localisation sequence.

In an embodiment, the hybridized third nucleic acid forms, together with the second (3′) constant sequence, a stretch of double stranded DNA containing a second restriction site for a second endonuclease. This second restriction site for a second endonuclease makes it possible to release from the labelled cells, or organelles, the nucleic acids comprising the localisation sequences or its complementary sequence that are bound thereto, by cleavage with said second endonuclease. This release may be implemented in a subsequent method of analysing the labelled individualised cells or organelles (see following section “Method of mapping and sequencing individual cells or organelles of a tissue sample”).

For example, where the second constant sequence comprises or consists of GTACGGCTCGATAAGCTAGCA (SEQ ID NO: 2), the third nucleic acid may comprise or consist of sequence TGCTAGCTTATCGAGC (SEQ ID NO: 5). Said pair of second constant sequence and third nucleic acid, when hybridised, form a stretch of double stranded DNA containing a restriction site for Bcll.

Preferably, in this embodiment, the stretch of double stranded DNA formed by the first (5′) constant sequence and second nucleic acid contains no restriction site for the second endonuclease, and the stretch of double stranded DNA formed by the second (3′) constant sequence and third nucleic acid contains no restriction site for the first endonuclease. For instance, the first constant sequence comprises or consists of ACCTGATCACCCTGTGCGCGTCA (SEQ ID NO: 1), the second nucleic acid comprises or consists of sequence CACAGGGTGATCAGGT (SEQ ID NO: 6), the second constant sequence comprises or consists of GTACGGCTCGATAAGCTAGCA (SEQ ID NO: 2), and the third nucleic acid comprises or consists of sequence TGCTAGCTTATCGAGC (SEQ ID NO: 5).

In some embodiments, the third nucleic acid is attached at its 5′ end, covalently or non-covalently (preferably covalently), to the ligand which binds to the receptor or receptors of cells or organelles. In some other embodiments, the third nucleic acid is attached at its 5′ end, covalently or non-covalently (preferably covalently), to a first member of a non-covalent interacting pair, such as biotin-streptavidin pair, preferably to biotin; in such as case the ligand which binds to the receptor or receptors of cells or organelles is attached to the other member of the non-covalent interacting pair, such as the biotin-streptavidin pair, preferably streptavidin.

In some embodiments where the second or the third nucleic acid are hybridized to the second constant region (3′ constant sequence), the second or the third nucleic comprises a hairpin-loop.

According to another aspect, and alternative method is implemented which involves 5′ anchoring of localisation nucleic acids. According to this aspect (see e.g. FIG. 14), the method for labelling individual cells or organelles within a tissue sample with a localisation nucleic acid comprises:
A) providing a nucleic acid array comprising a substrate and localisation nucleic acids, wherein each localisation nucleic acid comprises, from the 5′ end to the 3′ end:
   i. a first (5′) constant sequence;
   ii. a localisation sequence that is indicative of the position of the nucleic acid on the nucleic acid array; and
   iii. a second (3′) constant sequence;
wherein the localisation nucleic acid is attached, covalently or non-covalently, to the substrate of the nucleic acid array;
B) providing a tissue sample labelled with a ligand which binds to a receptor or receptors of cells or organelles in the tissue sample, wherein the ligand is attached, covalently or non-covalently, to a capture nucleic acid which comprises at its 3′ end a sequence complementary to all or part of said second (3′) constant sequence;
C) contacting the nucleic acid array with the tissue sample so that the localisation nucleic acids are at the interface between the nucleic acid array and the tissue sample;
D) hybridising the localisation nucleic acid and capture nucleic acid, and extending the 3′ end of capture nucleic acid by polymerisation, thereby synthesising a sequence complementary to said localisation nucleic acid;
E) releasing all or part of the localisation nucleic acids comprising all or part of the first (5′) constant sequence, the localisation sequence, and all or part of the second (3′) constant sequence;
F) dissociating the tissue sample and recovering individualized cells or organelles, wherein at least a sub-population of the individualised cells or organelles is labelled with the capture nucleic acid comprising a sequence complementary to the localisation sequence.

According to this aspect of the method, the hybridised capture nucleic acid forms, together with the first (3′) constant sequence of the localisation nucleic acid, a stretch of double stranded DNA containing a first restriction site for an endonuclease. The endonuclease is used in the method to release, from the substrate of the nucleic acid array, a part of the localisation nucleic acid hybridised to the capture nucleic acid bound to the ligand or member of the non-covalent interacting pair.

### 3′ Anchoring

The 3′ end of the localisation nucleic acid is attached, covalently or non-covalently, optionally via a spacer sequence and/or linker, to the substrate of the nucleic acid array. In an embodiment, the 3′ end of the localisation nucleic acid is attached, covalently or non-covalently, via a spacer sequence and/or linker, to the substrate of the nucleic acid array.

In the method for labelling individual cells within a tissue sample, the localisation nucleic acid is attached at step a), covalently or non-covalently, to the ligand which binds to the receptor or receptors of cells, or become attached at step d), covalently or non-covalently, to the ligand which binds to the receptor or receptors of cells or organelles.

In an embodiment, in step a), the localisation nucleic acid is attached at its 5′ end to the ligand which binds to the receptor or receptors of cells or organelles.

In another embodiment, in step a), the localisation nucleic acid is attached at its 5′ end to a first member of a non-covalent interacting pair, such as biotin-streptavidin pair, preferably to biotin (see e.g. FIG. 3). Step c) then comprises releasing all or part of the localisation nucleic acids comprising i) the first (5′) constant sequence, ii) the localisation sequence, (iii) all or part of the second (3′) constant sequence, and the first member of the non-covalent interacting pair attached at the 5′ end of the first (5′) constant sequence. For binding to the ligand which binds to the receptor or receptors of cells or organelles, at step d), a ligand is used which is attached to the other member of the non-covalent interacting pair, such as the biotin-streptavidin pair, preferably streptavidin.

In the method, step a) may comprise hybridizing a second nucleic acid by complementarity to all or part of the first constant sequence (5′ constant sequence). For the purpose of differentiating the different nucleic acids implemented in the method, said nucleic acid having complementarity with, or complementary to, all or part of the first constant sequence is called “second nucleic acid”. The second nucleic acid is preferably a DNA.

Preferably, the hybridized second nucleic acid forms, together with the first constant sequence, a stretch of double stranded DNA of at least 10 base pairs.

In an embodiment of the method, step a) comprises hybridizing a second nucleic acid by complementarity to all or part of the first constant sequence (5′ constant sequence), wherein the second nucleic acid is attached, covalently or non-covalently (preferably covalently), to the ligand which binds to a receptor or receptors of the cells or organelles in the tissue sample (see e.g. FIG. 11). Step c) then comprises releasing all or part of the localisation nucleic acids comprising i) the first (5′) constant sequence, ii) the localisation sequence, (iii) all or part of the second (3′) constant sequence, and the hybridised second nucleic acid attached to the ligand.

In another embodiment of the method, step a) comprises hybridizing a second nucleic acid by complementarity to all or part of the first constant sequence (5′ constant sequence), wherein the second nucleic acid is attached, covalently or non-covalently (preferably covalently), to a first member of a non-covalent interacting pair, such as biotin-streptavidin pair, preferably to biotin (see e.g. FIGS. 2 and 10). The hybridized second nucleic acid preferably forms, together with the first (5′) constant sequence, a stretch of double stranded DNA containing a first restriction site for a first endonuclease. Step c) then comprises releasing all or part of the localisation nucleic acids comprising i) the first (5′) constant sequence, ii) the localisation sequence, (iii) all or part of the second (3′) constant sequence, and the hybridised second nucleic acid attached to the first member of a non-covalent interacting pair. For binding to the ligand which binds to the receptor or receptors of cells or organelles, at step d), a ligand is used which is attached to the other member of the non-covalent interacting pair, such as the biotin-streptavidin pair, preferably streptavidin.

In certain embodiments, step a) comprises hybridizing a third nucleic acid by complementarity to all or part of the second constant sequence (3′ constant sequence) (see e.g. FIGS. 2-4, 6-7, 9-12). For the purpose of differentiating the different nucleic acids implemented in the method, said nucleic acid having complementarity with, or complementary to, all or part of the second constant sequence is called “third nucleic acid”, although there might be no “second nucleic acid” implemented in the method. The third nucleic acid is preferably a DNA.

Preferably, the hybridized third nucleic acid forms, together with the second (3′) constant sequence, a stretch of double stranded DNA containing a second restriction site for a second endonuclease (see e.g. FIGS. 2-4, 6-7, 9-12). The endonuclease may generate sticky ends, blunt ends, or hydrolyse only the strand of first constant sequence in the double stranded DNA (nicked DNA).

According to an embodiment, the stretch of double stranded DNA formed by the first (5′) constant sequence and second nucleic acid contains no restriction site for the second endonuclease, and the stretch of double stranded DNA formed by the second (3′) constant sequence and third nucleic acid contains no restriction site for the first endonuclease. For example, where the second (3′) constant sequence comprises or consists of GTACGGCTCGATAAGCTAGCA (SEQ ID NO: 2), the third nucleic acid may comprise or consist of sequence TGCTAGCTTATCGAGC (SEQ ID NO: 5). Said pair of second (3′) constant sequence and third nucleic acid, when hybridised, form a stretch of double stranded DNA containing a restriction site for Bmtl (as underlined in this example).

In an embodiment, the 3′ end of the hybridized third nucleic acid is extended using a non-strand displacing, template-dependent DNA polymerase and the nick between the extended third nucleic acid and the second nucleic acid is repaired by a ligase, thereby creating a fourth nucleic acid comprising, from the 5′ to 3′ end, the sequence of the third nucleic acid, the sequence complementary to that of the localization sequence, and the sequence of the second nucleic acid. In this embodiment the second nucleic acid is attached at its 3′ end, covalently or non-covalently, to i) the ligand which interacts, in the tissue sample, with a receptor or receptors at the surface, or inside the cells, including organelles, or to ii) a first member of a non-covalent interacting pair, such as biotin-streptavidin pair, preferably to biotin (see e.g. FIG. 10). In situation ii), use if made of a ligand which is attached to the other member of the non-covalent interacting pair, such as the biotin-streptavidin pair, preferably streptavidin, wherein the ligand interacts, in the tissue sample, with a receptor or receptors at the surface, or inside the cells, including organelles.

In step c), a part of the localisation nucleic acid comprising part of the second (3′) constant sequence, the localisation sequence, and the first (5′) constant sequence is released from the nucleic acid array by cleavage catalysed by the second endonuclease.

The second endonuclease is used in the method to release a part of the localisation nucleic acid from the substrate of the nucleic acid array. Accordingly, in step c), all or part of the localisation nucleic acid comprising i) the first (5′) constant sequence, ii) the localisation sequence, iii) all or part of the second (3′) constant sequence is released from the nucleic acid array by cleavage catalysed by the second endonuclease. In embodiments wherein the hybridised third nucleic acid is attached at its 5′ end to a first member of a non-covalent interacting pair, or to the ligand, the released localisation nucleic acid is hybridised to the third nucleic acid bound to the first member of a non-covalent interacting pair or ligand.

According to an embodiment, a ligand is used which is covalently or non-covalently attached to a capture nucleic acid comprising a region fully or partly complementary to the first (5′) constant sequence (see e.g. FIG. 4). In step d), the released localisation nucleic acid comprises i) the first (5′) constant sequence, ii) the localisation sequence, and iii) all or part of the second (3′) constant sequence, wherein the first (5′) constant sequence is hybridised to the complementary region of the capture nucleic acid. Step d) further includes (1) extending the 3′ end of the capture nucleic acid and the 3′ end of the complementary region of the capture nucleic acid, using a template-dependent DNA polymerase (e.g. phi29), thereby forming an extended double-stranded nucleic acid, and (2) removing 5′ mononucleotides from the extended double-stranded nucleic acid, using an exonuclease acting in the 5′ to 3′ direction (e.g. T7 exonuclease).

According to another embodiment, a ligand is used which is covalently or non-covalently attached to a capture nucleic acid comprising a strand complementary to part of the first (5′) constant sequence (see e.g. FIG. 6). Step d) comprises (1) using a template-dependent non-strand displacing DNA polymerase (e.g. Phusion) to synthesise a strand complementary to the released localisation nucleic acids comprising i) the first (5′) constant sequence, ii) the localisation sequence, and iii) all or part of the second (3′) constant sequence, thereby forming partly or fully doubled stranded nucleic acids, (2) removing partly 5′ mononucleotides using an exonuclease acting in the 5′ to 3′ direction (e.g. T7 exonuclease) thereby producing partly doubled stranded localisation nucleic acids with stick ends, and (3) ligating the localisation nucleic acids with the capture nucleic acids.

In another embodiment, the third nucleic acid forms, together with the second constant sequence, a stretch of double stranded DNA containing a second restriction site for a second endonuclease, and the double stranded DNA when cleaved by the second endonuclease forms a ligation site (see e.g. FIG. 7) or releases all or part of the localisation nucleic acid (FIG. 8). The second endonuclease is used in step c) to release all or part of the localisation nucleic acid from the substrate of the nucleic acid array, wherein released localisation nucleic acids comprise i) the first (5′) constant sequence, ii) the localisation sequence, and iii) all or part of the second (3′) constant sequence hybridised to all or part of the third nucleic acid thereby forming a ligation site. For binding the released localisation nucleic acids to cells of the tissue sample via the ligand bound to a receptor or receptors of cells or organelles, a ligand is used which is covalently or non-covalently attached to a partly doubled stranded capture nucleic acid, wherein the protruding strand is complementary to the non-hybridised region of the second constant sequence. Step d) further includes repairing with a ligase the nick between partly doubled stranded capture nucleic acid and the partly doubled stranded formed by the third nucleic acid is hybridised to the second constant sequence of the released localisation nucleic acids.

In another embodiment, the third nucleic acid forms, together with the second constant sequence, a stretch of double stranded DNA containing a second restriction site for a second endonuclease which is a nicking endonuclease (see e.g. FIG. 9). Step a) further comprises using a strand displacing, template-dependent DNA polymerase (e.g. phi29) to synthesise a strand complementary to at least part of the second (3′) constant sequence, the localisation sequence and the first (5′) constant sequence. Step c) comprises releasing the nucleic acid complementary to the localisation nucleic acid by cleaving with the nicking endonuclease and strand-displacement by the template-dependent DNA polymerase that primes at the nick, and step d) comprises hybridising the released complementary nucleic acid to a capture nucleic acid, covalently or non- covalently bound to the ligand, which comprises at least part of the second constant sequence.

According to certain aspects, the hybridized second nucleic acid forms, together with the first (5′) constant sequence, a stretch of double stranded DNA containing a first restriction site for a first endonuclease. Preferably, the stretch of double stranded DNA formed by the first (5′) constant sequence and second nucleic acid contains no restriction site for the second endonuclease, and the stretch of double stranded DNA formed by the second (3′) constant sequence and third oligonucleotide contains no restriction site for the first endonuclease. This first restriction site for a first endonuclease makes it possible to release from the labelled cells, or organelles, the nucleic acids comprising the localisation sequences or its complementary sequence that are bound thereto, by cleavage with said first endonuclease. This release may be implemented in a subsequent method of analysing the labelled individualised cells or organelles (see following section “Method of mapping and sequencing individual cells or organelles of a tissue sample”).

According to a variant of the above embodiments, a chemically- or photo-cleavable link is used instead of a stretch of double stranded DNA containing a restriction site for an endonuclease.

In some embodiments where the second or the third nucleic acid are hybridized to the first constant region (5′ constant sequence), the second or the third nucleic comprises a hairpin-loop.

According to another aspect (see e.g. FIG. 5), an alternative method is implemented which involves 3′ anchoring of localisation nucleic acids. According to this aspect, the method for labelling individual cells or organelles within a tissue sample with a localisation nucleic acid comprises:
A) providing a nucleic acid array comprising a substrate and localisation nucleic acids, wherein each localisation nucleic acid comprises, from the 5′ end to the 3′ end:
   i. a first (5′) constant sequence;
   ii. a localisation sequence that is indicative of the position of the nucleic acid on the nucleic acid array; and
   iii. a second (3′) constant sequence;
wherein the localisation nucleic acid is attached, covalently or non-covalently, to the substrate of the nucleic acid array;
B) providing a tissue sample labelled with a ligand which binds to a receptor or receptors of cells or organelles in the tissue sample, wherein the ligand is attached, covalently or non-covalently, to a capture nucleic acid which comprises at its 3′ end a sequence complementary to all or part of said second (3′) constant sequence;
C) contacting the nucleic acid array with the tissue sample so that the localisation nucleic acids are at the interface between the nucleic acid array and the tissue sample;
D) hybridising the localisation nucleic acid and capture nucleic acid, and extending the 3′ end of capture nucleic acid by polymerisation, thereby synthesising a sequence complementary to said localisation nucleic acid;
E) releasing all or part of the localisation nucleic acids comprising all or part of the first (5′) constant sequence, the localisation sequence, and all or part of the second (3′) constant sequence;
F) dissociating the tissue sample and recovering individualized cells or organelles, wherein at least a sub-population of the individualised cells or organelles is labelled with the capture nucleic acid comprising a sequence complementary to the localisation sequence.

According to this aspect of the method, the hybridised capture nucleic acid forms, together with the first (3′) constant sequence of the localisation nucleic acid, a stretch of double stranded DNA containing a first restriction site, which is a nicking site for a nicking endonuclease. The nicking endonuclease is used in the method to release, from the substrate of the nucleic acid array, a part of the localisation nucleic acid hybridised to the capture nucleic acid bound to the ligand or member of the non-covalent interacting pair.

### Kits for Labelling Individual Cells or Organelles

It is further provided a kit for labelling individual cells or organelles within a tissue sample with a localisation nucleic acid sequence, which comprises:
a) a nucleic acid array comprising localisation nucleic acids, wherein each localisation nucleic acid comprises, from the 5′ end to the 3′ end:
   a first constant sequence;
   a localisation sequence that is indicative of the position of the nucleic acid on the nucleic acid array; and
   a second constant sequence;
wherein the localisation nucleic acid is attached, covalently or non-covalently, to the substrate of the nucleic acid array;
b) at least one type of ligand that binds to a receptor of cells or organelles in the tissue, preferably a ubiquitously receptor present at the surface of the cells or organelles of a tissue sample, wherein the at least one type of ligand is optionally attached to a member of a non-covalent interacting pair; and
c) optionally both members of the non-covalent interacting pair.

According to an embodiment, the ligand is in free form, i.e. it is not attached to a member of a non-covalent interacting pair (e.g. biotin-streptavidin). The kit then does not contain the other member of non-covalent interacting pair. According to another embodiment, the kit comprises the ligand and both members of the non-covalent interacting pair; and the ligand is preferably provided attached to one member of the non-covalent interacting pair, preferably to streptavidin.

According to still another embodiment, the ligand is directly attached to the localisation nucleic acid, or to a nucleic acid that hybridises to the localisation nucleic acid.

According to an embodiment, the localisation nucleic acid is attached at the 3′ end, covalently or non-covalently, to the substrate of the nucleic acid array and the kit further comprises:
a second nucleic acid, to which is attached, covalently or non-covalently, a ligand which interacts, in the tissue sample, with a receptor or receptors at the surface, or inside the cells or organelles, that forms, together with the first (5′) constant sequence, a stretch of double stranded DNA containing a first restriction site for a first endonuclease
a third nucleic acid complementarity to all or part of the second constant sequence that forms, together with the second constant sequence, a stretch of double stranded DNA containing a second restriction site for a second endonuclease
said second endonuclease which is capable of cleaving a restriction site in a double stranded DNA formed by the third nucleic acid and second constant sequence, when hybridized; and
optionally said first endonuclease which is capable of cleaving a restriction site in a double stranded DNA formed by the second oligonucleotide and first constant sequence, when hybridized, wherein the stretch of double stranded DNA formed by the first constant sequence and second oligonucleotide contains no restriction site for the second endonuclease, and the stretch of double stranded DNA formed by the second constant sequence and third oligonucleotide contains no restriction site for the first endonuclease.

According to another embodiment, the localisation nucleic acid is attached at the 5′ end, covalently or non-covalently, to the substrate of the nucleic acid array and the kit further comprises:
a second nucleic acid complementarity to all or part of the first constant sequence that forms, together with the first constant sequence, a stretch of double stranded DNA containing a first restriction site for a first endonuclease
optionally, a third nucleic acid complementarity to all or part of the second constant sequence that forms, together with the second constant sequence, a stretch of double stranded DNA containing a second restriction site for a second endonuclease
optionally a first endonuclease which is capable of cleaving a restriction site in a double stranded DNA formed by the second nucleic acid and first constant sequence, when hybridized;
a second endonuclease which is capable of cleaving a restriction site in a double stranded DNA formed by the third oligonucleotide and second constant sequence, when hybridized, wherein the stretch of double stranded DNA formed by the first constant sequence and second oligonucleotide contains no restriction site for the second endonuclease, and the stretch of double stranded DNA formed by the second constant sequence and third oligonucleotide contains no restriction site for the first endonuclease

According to still another embodiment, the kit further comprises said second nucleic acid, first endonuclease, third nucleic acid, and second endonuclease.

In the above embodiments, the kit may further comprise a capture nucleic acid which captures the released localisation nucleic acid, or its complementary nucleic acid, by hybridisation or ligation. Preferably, the capture nucleic acid is non-covalently bound to the ligand, via the member of the non-covalent interacting pair.

The kit may also further comprise a polymerase, ligase, exonuclease, and/or any other enzyme as used in a method of the invention.

The nucleic acid array provided in the kit can be pre-hybridized with the first and the second oligonucleotide.

### Method of Spatial Mapping and Sequencing Individual Cells or Organelles of a Tissue Sample

In the above method, the hybridized third nucleic acid forms, together with the second (3′) constant sequence, a stretch of double stranded DNA containing a second restriction site for a second endonuclease. This second restriction site for a second endonuclease makes it possible to release from the labelled cells, or organelles, the nucleic acids comprising the localisation sequences or its complementary sequence that are bound thereto, by cleavage with said second endonuclease. This release may be implemented in a subsequent method of analysing the labelled individualised cells or organelles.

It is further provided a method of spatial mapping and sequencing individual cells or organelles of a tissue sample, the method comprising:
a) providing individualized cells labelled with a nucleic acid comprising a localisation sequence as obtainable or obtained by the method of labelling of the invention;
b) trapping the individualized cells or organelles labelled with the nucleic acid comprising the localisation sequence in a compartment (i.e. one or more compartments), wherein the compartment comprises a compartment-specific nucleic acid comprising a compartment-specific sequence and one or any combination of the following sequences for nucleic acid labeling and further sequencing: hybridization site, ligation site or recombination site
c) Optionally analyzing captured cells, organelles and/or molecules they secrete, using optical detection;
d) Optionally, lysing trapped cells, or cells and organelles, thereby releasing nucleic acids from the cells or organelles in the compartments;
e) Associating the compartment-specific sequence with the nucleic acids released from the cell or organelle in the compartments and the nucleic acids comprising the localisation sequence;
f) Recovering the nucleic acids produced at step e) from the compartments and sequencing recovered nucleic acids; and
g) Defining sequenced nucleic acids comprising the same compartment-specific sequence as originating from the same single cell or organelle, and mapping the position of the single cell originally on the tissue sample based on the localisation sequence(s) contained in part of the nucleic acid that comprise said same compartment-specific sequence, thereby combining mapping and sequencing information of the individual cells of the tissue sample.

Optionally, the method further comprises step h) of mapping the sequencing information back onto an image from microscopy, optionally fluorescent microscopy, of the tissue, taken before dissociation.

According to an embodiment, the optional step c) and/or d) is implemented in the above method of spatial mapping and sequencing.

In the frame of the method of mapping and sequencing individual cells or organelles of a tissue sample, the “individualized cells labelled with a nucleic acid comprising a localisation sequence” as obtainable by the method labelling of the invention, are actually labelled with a nucleic acid comprising the localisation sequence or its complementary sequence (as the complementary sequence still constitutes a sequence . indicative of the position of the nucleic acid on the nucleic acid array). Cellular nucleic acids and localisation sequences carrying the same compartment-specific sequence are derived from the same single-cell, allowing single-cell “omic” date (genomic, epigenomic, transcriptomic, proteomic) to be mapped onto the original position of the cell in the tissue.

As used herein, the term “compartments” denotes for instance droplets, microfabricated chambers separated by pneumatic valves, microfabricated chambers made with actuatable hydrogels, microfabricated wells, actuatable hydrogel cages or microplate wells.

According to an embodiment, the method of mapping and sequencing individual cells or organelles comprises implementing the method labelling of the invention to provide individualized cells or organelles labelled with a nucleic acid comprising the localisation sequence (or its complementary sequence as explained above).

In an embodiment of the method of mapping and sequencing individual cells or organelles, single cells or single organelles are trapped in a compartment.

Preferably, the nucleic acids comprised in the compartment are DNAs.

The compartment may comprise a plurality of compartment-specific nucleic acids for targeting specifically different nucleic acids. Preferably, the compartment-specific nucleic acids comprise a barcode specific to the compartment.

In an embodiment of the method, step c) is implemented and the optical detection includes imaging, including fluorescence imaging, or fluorescence detection for instance.

Reads corresponding to cellular nucleic acids and localisation sequences carrying the same compartment-specific sequence are derived from the same single-cell, allowing single-cell “omic” data (genomic, epigenomic, transcriptomic, proteomic) to be mapped onto the original position of the cell in the tissue.

According to an embodiment, triangulation of the localisation sequences carrying the same compartment-specific sequence is used to assign a unique relative position in the tissue per cell.

The compartment-specific nucleic acids may comprise, for example, a 3′-region of sequence oligo d(T) or oligo d(T)VN, for hybridization to the poly(A) tail of mRNA (for mRNA sequencing), a 3′-region of sequence complementary to that of a specific RNA (for targeted RNA sequencing) or DNA (for targeted DNA sequencing), or a 3′-region of random sequence, for example d(N)6 (for RNA sequencing or DNA sequencing). More specifically, it is a constant region of a primer containing a compartment-specific sequence that hybridizes to the released nucleic acids.

More specifically, then step e) of associating the compartment-specific sequence with either the nucleic acids released from the cell or organelle in the compartments and the nucleic acids comprising the localisation sequence comprises:
i) hybridizing the nucleic acids containing the compartment-specific sequence by complementarity to the released nucleic acids from the cell or organelle and extending the compartment-specific nucleic acid hybridized to the released nucleic acids using a DNA polymerase to create the complementary strand of the released nucleic acids with an associated compartment-specific sequence; or
ii) hybridizing the nucleic acids containing the compartment-specific sequence to the localisation nucleic acid associated with the cell or organelle and extending one or both hybridized DNA strands using a DNA polymerase to create a DNA molecule comprising both the localisation sequence, or it's complement, and the compartment-specific sequence, or its complement.

According to an embodiment, the compartment-specific nucleic acids comprising a primer sequence complementary to all or part of the second constant sequence present in the localisation nucleic acids are bound to individualized cells or organelles by ligand-receptor pairing. In this embodiment, step e) may further comprise extending the DNA strands hybridized to the localisation nucleic acids using a DNA polymerase to create the complementary strand of the localisation nucleic acids associated with a compartment specific barcode.

In some embodiments, step e) additionally comprises:
hybridizing the compartment-specific nucleic acid by complementarity to the 3′-end of cDNA produced by reverse transcription of RNA from the cell or organelle and extending the cDNA hybridized to the compartment-specific nucleic acid using a DNA polymerase to create the complementary strand of the compartment-specific nucleic acid with an associated compartment-specific sequence;
ligating the compartment-specific nucleic acid to the DNA present in the compartment; or
recombining the compartment-specific nucleic acid with the DNA present in the compartment.

The method of mapping and sequencing individual cells or organelles may comprise additional steps inserted between c) and d) or e) and f), comprising the release of the compartment-specific nucleic acids in the compartment.

According to another embodiment, the compartment-specific nucleic acids comprising a barcode are DNA, and may be wholly or partly double-stranded, and the method may comprises ligating the DNA comprising a barcode to DNA released by the cells or organelles and the nucleic acids comprising the localisation sequence. For example, the barcodes may be ligated to genomic DNA, for example after restriction digestion (for genomic DNA sequencing or analysis of DNA methylation), or after digestion with micrococcal nuclease (for metagenomic analysis using MNase-seq or ChIP-seq).

According to still another embodiment, the nucleic acids comprising a barcode are DNA, and may be wholly or partly double-stranded, and the method comprises recombining the DNA comprising barcode with DNA released by the cells or organelles and the nucleic acids comprising the localisation sequence. For example, the barcodes may be recombined with genomic DNA for genomic DNA sequencing, or epigenomic analysis of DNA methylation (using Methyl-seq or bisulfite sequencing) or chromatin structure (using transposase-accessible chromatin with sequencing, ATAC-Seq). Alternatively, the nucleic acids comprising a barcode recombine with RNA-DNA duplexes formed after first strand cDNA synthesis on RNA released by the cells or organelles and the nucleic acids comprising the localisation sequence, or recombining with double-stranded DNA formed after first and second strand cDNA synthesis on RNA released by the cells or organelles (for RNA sequencing) and the nucleic acids comprising the localisation sequence. In a preferred embodiment the oligonucleotide comprises a Mosaic End (ME) sequence which recombines with DNA catalyzed by Tn5 transposase.

According to an embodiment, the method further comprises releasing the nucleic acids comprising barcodes upon the presence of a cellular or organellar material (e.g. a surface molecule, a secreted molecule, or a lysis product) in the compartments, e.g. by a proximity ligation assay, or proximity extension assay.

Reads corresponding to cellular nucleic acids and localisation sequences carrying the same compartment-specific sequence are derived from the same single-cell, allowing single-cell “omic” date (genomic, epigenomic, transcriptomic, proteomic) to be mapped onto the original position of the cell in the tissue.

According to an embodiment, the compartments are droplets and individualized cells labelled with a nucleic acid comprising a localization sequence are encapsulated in a droplet together with single beads carrying nucleic acids with a bead-specific sequence (i.e. the compartment-specific nucleic acids).

According to an embodiment, the compartments are wells of a microplate, wherein each well comprise a plurality of oligonucleotides, said oligonucleotides comprising a compartment specific sequence specific to the well, and individualized cells labelled with a nucleic acid comprising a localisation sequence are trapped in a well.

According to another embodiment, the compartments are microfabricated chambers made with actuatable hydrogels. In this embodiment of microfabricated chambers made with actuatable hydrogels, the compartments are preferably compartments of a microfluidic device as defined hereafter.

### Microfluidic Device

In this embodiment, the compartments are compartments of a microfluidic device comprising:
a first wall comprising a first substrate on which a plurality of closed patterns is grafted,
a second wall, facing the first wall, comprising a second substrate,
a plurality of nucleic acids grafted either on the first substrate or on the second substrate, each nucleic acid comprising a barcode that encodes the position of the nucleic acid on said first or second substrate.
wherein at least the plurality of closed patterns or the second substrate is made of an actuatable hydrogel which is swellable between a retracted state and a swollen state in which the closed patterns and the second substrate come into contact.

In the swollen state, the closed patterns and the second substrate of the device are in contact. The device thus comprises a plurality of cages, each cage being delimited by a lateral wall made of the closed patterns and by end walls constituted of the first and the second substrates.

In the retracted state, the closed patterns and the second substrate are no more in contact. A gap between the closed patterns and the second substrate allows fluids and cells freely circulating inside the device.

Between the retracted and the swollen states, the device according to the invention goes through a multitude of intermediary states wherein the actuatable hydrogel is only partially swollen. In these configurations, a gap between the closed patterns and the second substrate still exists. However, the height of the gap is sufficiently reduced with respect to the retracted state so that cells, captured in the cages, are retained in the cages. These intermediary configurations may typically be used to allow a selective passage of fluids but not cells.

Each closed pattern thus defines a trapping site for cells wherein closure and opening are initiated by an external stimulus. In a preferred embodiment, the external stimulus is a change in pH, in light intensity, in temperature or in electrical current intensity. In a highly preferred embodiment, the external stimulus is a change in temperature.

The microfluidic device comprises at least 2 closed patterns. Preferably, the microfluidic device comprises a large number of closed patterns, typically 100, 1,000, 10,000, 100,000 . . .

The first wall and the second wall are made of a rigid material that is capable of resisting temperature fluctuations ranging from −20 to 100° C. According to a first embodiment, the walls (first and/or second walls) are made of a unique and homogenous material. The wall thus consists of the substrates.

According to a second embodiment, the walls further comprise a support material on which is fixed/coated the substrate. Typically, the wall consists of a support material made of glass or polydimethylsiloxane, covered with a substrate layer.

The microfluidic device may equivalently comprise two monolayer walls, two multilayer walls or one monolayer wall and one multilayer wall.

The first substrate is typically made of a material chosen from: silicon, quartz, glass, polydimethylsiloxane, thermoplastics such as cyclic olefin copolymers and polycarbonates, preferably from glass and polydimethylsiloxane. Preferably, the first substrate is made of polydimethylsiloxane.

According to an embodiment, at least part of the surface of the first substrate is structured and/or functionalized.

By “structured”, it is meant that the surface of the substrate is irregular. The surface of the substrate may be porous or microscopically structured. In particular, it can comprise microscopic streaks, pillars, etc . . .

The structuration of the substrate may be performed according to any known process. Mention may for example be made of standard soft-lithography techniques which are well documented.

By “functionalized”, it is meant that the fixation of chemical functional groups on the surface of the substrate. Typically, the surface of the first substrate is functionalized with chemical groups chosen from hydroxide groups, silanol groups and mixtures thereof, preferably silanol groups.

The structuration and/or the functionalization of the substrate(s) permit to facilitate the grafting of the closed patterns and/or of the nucleic acids on their surface.

According to a preferential embodiment, the first wall is made of a structured and/or functionalized polydimethylsiloxane substrate, preferably of a structured and functionalized polydimethylsiloxane substrate.

The closed patterns may have a large variety of shape. Preferably, the closed patterns are rectangular, square, circular or hexagonal.

Preferably, the closed patterns are covalently grafted to the first substrate.

According to a particular embodiment, the second substrate is made of the hydrogel and the closed patterns are made of a non-swellable material. Preferably, according to this particular embodiment, the second wall comprises a non-swellable support material on which is deposited the swellable hydrogel. The non-swellable support material may be structured and/or functionalized. Structuration and/or functionalization of the non-swellable material are made by analogy with what has been said above in the context of the first substrate. Thus, according to this embodiment, the closed patterns are non-swellable and it is the swelling of the second substrate that permits the closure of the cages.

Preferably, according to this embodiment, the closed patterns are made of a material chosen from silicon, quartz, glass, polydimethylsiloxane, thermoplastic materials such as cyclic olefin copolymers and polycarbonates, preferably from glass or polydimethylsiloxane.

Preferably, the closed patterns have a height ranging from 0.1 to 100 μm, preferably from 1 to 30 μm.

Preferably, the walls of closed patterns have a thickness ranging from 0.1 to 500 μm, preferably from 1 to 20 μm.

Advantageously, according to this embodiment, the second substrate has a thickness, measured in the swollen state into contact with the closed patterns, ranging from 1 to 500 μm, preferably from 1 to 100 μm.

Advantageously, still according to this embodiment, the second substrate comprising the hydrogel has a thickness, measured in the dry state, ranging from 0.5 to 150 μm, preferably from 0.5 to 50 μm.

According to a preferred embodiment, the closed patterns are made of the hydrogel and the second substrate is made of a non-swellable material. Thus, according to this embodiment, the second substrate is non-swellable and the closed patterns swell to close the cages.

Preferably, according to this preferred embodiment, the second substrate is made of a material chosen from: silicon, quartz, glass, polydimethylsiloxane, thermoplastics such as cyclic olefin copolymers and polycarbonates, preferably from glass or polydimethylsiloxane. Advantageously, the hydrogel patterns have a height, measured in the swollen state when the hydrogel patterns are in contact with the second wall, ranging from 0.1 μm to 500 μm, preferably from 1 μm to 250 μm, more preferably from 1 μm to 100 μm.

Advantageously, the hydrogel pattern has a height, measured in the dry state, ranging from 0.1 μm to 150 μm, preferably from 0.5 μm to 100 μm, more preferably from 0.5 μm to 50 μm.

Preferably, the walls of the hydrogel patterns have a resolution, measured in the swollen state when the hydrogel patterns are in contact with the second wall, ranging from 0.1 μm to 100 μm, preferably from 1 μm to 10 μm.

Preferably, the walls of the hydrogel patterns have a resolution, measured in the dry state, ranging from 0.1 μm to 100 μm, preferably from 0.5 μm to 5 μm.

By “hydrogel”, we refer in the context of a gel comprising a polymer matrix forming a three-dimensional network which is capable of swelling in the presence water, under specific physico-chemical conditions. The swelling of the hydrogel may for example be initiated by a thermal, optical, chemical or electrical stimulus.

For example, the swelling (or the deflation) of the hydrogel may be initiated by a change in temperature, in pressure or in the pH value of the medium wherein it is placed.

Preferably, the hydrogel is a temperature-responsive swellable hydrogel. By “temperature-responsive swellable hydrogel”, we refer in the context of the invention to a hydrogel which swelling or deflation is induced by varying the temperature. A temperature-responsive swellable hydrogel typically exhibits a drastic change of water-solubility with temperature.

In a specific range of temperature, the hydrogel is water-soluble and absorb large quantities of water.

Reversely, by changing the temperature of the medium, the hydrogel becomes no more water-soluble. The hydrogel then releases water and deflates.

By “swollen state”, we refer in the context of the invention to a state of the hydrogel wherein the closed patterns and the second substrate are in contact such that the device comprises a plurality of hermetically sealed cages.

By “retracted state”, we refer in the context of the invention to a state of the hydrogel wherein the closed patterns and the second substrate are not in contact: a gap between the closed patterns and the second substrate exists and permits a free circulation of fluids and cells inside the microfluidic device. The “retracted state” differs from the “dry state” define below in that the hydrogel is not completely free from water. In the retracted state, the hydrogel is still at least partially hydrated.

By “dry state”, we refer in the context of the invention to a state wherein the hydrogel is almost completely free from water. Typically, the hydrogel is in the dry state during the manufacture of the microfluidic device, notably during the grafting of the hydrogel patterns of during the coating of the second wall with the hydrogel substrate.

The temperature at which the water-solubility properties of the hydrogel drastically change is designated as the critical solution temperature (CST).

Preferably, the hydrogel has a critical solution temperature (CST) ranging from 4° C. to 98° C., more preferably from 20° C. to 50° C., even more preferably from 25° C. to 40° C.

According to a first variant, the critical solution temperature (CST) of the hydrogel is a lower critical solution temperature (LCST). At a temperature superior to the LSCT, the hydrogel is in the retracted state and at a temperature inferior to the LCST, the hydrogel is in the swollen state.

According to a second variant, the critical solution temperature (CST) of the hydrogel is an upper critical solution temperature (UCST). At a temperature superior to the UCST, the hydrogel is in the swollen state and at a temperature inferior to the USCT, the hydrogel is in the retracted state.

The polymer constituting the polymer matrix of the hydrogel is typically chosen from homopolymers, copolymers and terpolymers of acrylics, alkyl (meth)acrylates, alkyl (meth)acrylamides, oligoethylene (meth)acrylates, sulfobetaines (meth)acrylates and N-acryloyl glycinamide, preferably chosen from homopolymers copolymers and terpolymers of alkyl (meth)acrylamides and any mixtures thereof, more preferably the hydrogel comprises poly(N-Isopropylacrylamide).

The polymer may be chosen from LCST polymers, UCST polymers and mixtures thereof.

By analogy with what has been said above in the context of the hydrogel:
the expression “LCST polymer” designates a thermo-responsive polymer having a lower critical solution temperature, and
the expression “UCST polymer” designates a thermo-responsive polymer having an upper critical solution temperature.

The overall behavior of the hydrogel (UCST and/or LCST behavior) depends on the nature and on the amount of the different polymers present in the hydrogel.

When the polymer is chosen from UCST polymers, it is preferably chosen from homopolymers, copolymers and terpolymers of acrylics, alkyl (meth)acrylates, alkyl (meth)acrylamides, oligoethylene (meth)acrylates, sulfobetaines (meth)acrylates, N-acryloyl glycinamide and mixtures thereof.

Preferably, the UCST polymer is a terpolymer of methacrylamide, acrylamide and allylmethacrylate.

When the polymer is chosen from LCST polymers, it is preferably chosen from homopolymers, copolymers and terpolymers of acrylics, alkyl (meth)acrylates, alkyl (meth)acrylamides, oligoethylene (meth)acrylates and mixtures thereof, more preferably from homopolymers, copolymers and terpolymers of alkyl (meth)acrylamides, event more preferably the LCST polymer is poly(N-Isopropylacrylamide).

Preferably, the LCST polymer is poly(N-Isopropylacrylamide).

Advantageously, the polymer comprises, preferably consists of, one or several UCST or LCST polymers.

Advantageously, the microfluidic device further comprises at least one inlet and at least one outlet permitting respectively the introduction and the removal of reactants into the device.

Preferably, heating means are integrated in the device according to the invention.

According to an embodiment, each cage comprises independent heating means. This embodiment is particularly advantageous in that it permits to open and close each cage independently.

For example, local heating means may consist of nanoparticles, which heat up when irradiated with light (Plasmonic effect). The nanoparticles may for example be deposited between the hydrogel and the wall on which it is coated or dispersed in the polymer matrix of the hydrogel. Preferably, the nanoparticles are chosen from metal nanoparticles and plasmonic nanoparticles, preferably comprises gold, graphene, silver, copper and titanium nitride.

In another example, local heating is performed using microresistors; for example microresistors comprising chromium/gold bilayer or TiO₂ structures.

The microfluidic device further comprises a plurality of nucleic acids grafted either on the first substrate or on the second substrate, wherein each nucleic acid comprises a sequence barcode that encodes the position of the nucleic acid on said first or second substrate.

Advantageously, the nucleic acids are grafted so as to be placed inside the cages, when the hydrogel is in the swollen state.

More advantageously, the nucleic acids are grafted either on the first substrate, inside the closed patterns or on the second substrate, opposite the closed patterns.

Preferably, when the closed patterns are made of the hydrogel, the nucleic acids are grafted on the surface of the second substrate. Preferably, when the second substrate is made of the hydrogel, the nucleic acids are grafted on the surface of the first substrate.

The grafted nucleic acids are RNA or DNA, preferably DNA. The grafted nucleic acids can be single-stranded, double-stranded or partially double-stranded.

The grafted nucleic acids are preferably 60 to 100 nucleotide long.

The grafted nucleic acid may be attached to the substrate at the 3′-end or 5′-end, either directly, or by a linker.

According to an embodiment, grafted nucleic acids sharing the same barcode have a plurality of sequences. According to another embodiment, grafted nucleic acids sharing the same barcode have a same sequence.

According to an embodiment, all or part of the grafted nucleic acids are hybridized to another nucleic acid or a plurality of nucleic acids and form a partly or fully double stranded DNA, double stranded DNA/RNA, or double stranded RNA.

According to an embodiment, the grafted nucleic acids comprise one or any combinations of the following sequences:
1) a restriction site or a photocleavable site for nucleic acid release,
2) a sequence complementary to an amplification primer for further amplification,
3) a T7 RNA polymerase promoter sequence for further in vitro transcription (IVT),
4) a hybridization site for nucleic acid labeling, a ligation site for nucleic acid labeling or a recombination site for nucleic acid labeling, and
5) a sequence of randomized nucleotide residues that function as a unique molecular identifier (UMI).

Preferably, the grafted nucleic acids comprise at least i) a sequence barcode that encodes the position of the nucleic acid on said first or second substrate, and ii) a restriction site or a photocleavable site, and optionally further iii) a primer sequence, and/or T7 sequence and/or hybridization, ligation or recombination site.

According to an embodiment, the grafted nucleic acids of the microfluidic device comprise a constant sequence, i.e. a sequence which is present in all grafted nucleic acids. Grafted nucleic acids of the microfluidic device may be hybridized to a DNA comprising a sequence complementary to all or part of the constant sequence of the grafted nucleic acids. One, or a plurality of different DNA comprising a sequence complementary to all or part of the constant sequence, may be hybridized to the grafted nucleic acids.

The microfluidic device may further comprise structures capable of capturing a cell or an organelle. Such structures are typically chosen from publications as such as Vigneswaran N. et al, 2017, Microfluidic hydrodynamic trapping for single cell analysis: mechanisms, methods and applications, Anal. Methods,9, 3751-3772.

Preferably, the structures capable of capturing a cell or an organelle are localized either on the first substrate, inside the closed patterns or on the second substrate, opposite the closed patterns

Preferably, each cage comprises at least one structure capable of capturing a cell or an organelle.

According to a particular embodiment, a plurality of ligands is grafted, directly or indirectly, covalently or non-covalently, on the first substrate (14) and/or on the second substrate (20), opposite the closed patterns.

Advantageously, the ligands are grafted so as to be placed inside the cages, when the hydrogel is in the swollen state.

In particular, when grafted on the first substrate (14), the ligands are typically grafted inside the closed patterns (16).

Alternatively, when grafted on the second substrate (20), the ligands are facing the closed patterns.

The ligands may all be grafted on the same substrate. Alternatively, some of the ligands are grafted on the first substrate (14) and the others are grafted on the second substrate (20).

Preferably, when grafted directly on the first (14) or second (20) substrate, the plurality of ligands is covalently grafted on the first (14) or second (20) substrate.

According to a more specific embodiment, the plurality of ligands is grafted indirectly: the plurality of ligands is grafted to an intermediate structure, said intermediate structure being directly grafted on the first (14) or second (20) substrate. Thus, according to this specific embodiment, there is no direct bonding between the plurality of ligands and the substrates (14, 20).

Preferably, when grafted indirectly on the first (14) or second (20) substrate, the plurality of ligands is grafted non-covalently on the first (14) or second (20) substrate.

According to first example, the plurality of ligands is conjugated with a nucleic acid and is associated by hybridization to at least part of the grafted nucleic acids (22).

According to another example, the plurality of ligands are non-covalently grafted to an adhesion coating previously coated on the first (14) or second (20) substrate. As adhesion coatings, mentions may notably be made to streptadivin coatings.

In these embodiments, preferably, each ligand is independently chosen from the group consisting of antibodies, fragments of antibody, lectins, and aptamers.

The ligands are usually selected to bind one or more analyte(s) secreted or released by lysis of the cell(s) or organelles trapped in the cage formed by the first (14) and second (20) walls of the microfluidic device (10), and the closed pattern (16) of hydrogel in swollen state.

Said microfluidic device may be manufactured by a method that comprises the following steps:
1) Providing a first substrate,
2) Grafting on the surface of said first substrate a plurality of closed patterns,
3) Providing a second substrate,
4) Grafting a plurality of nucleic acids either on the surface of the first substrate, or on the surface of the second substrate, wherein each nucleic acid comprises a barcode that encodes the position of the nucleic acid on said first or second substrate;
5) Positioning the first substrate and the second substrate by placing the closed patterns and the nucleic acids between the first substrate and the second substrate,
6) Bonding the first and the second substrates.

The grafting of the closed patterns may be performed according to any known process.

When the closed patterns are made of a non-swellable material, the grafting of the closed patterns is typically performed by soft-lithography techniques.

According to a particular embodiment, the first substrate and the closed patterns are prepared together in a one and unique step.

When the closed patterns are made of the hydrogel, the grafting of the closed pattern is typically performed by photopatterning, preferably under UV (Ultraviolet) radiation. Photopatterning methods consists in the surface-grafting of the polymer matrix of hydrogel on the first substrate, and simultaneously by the crosslinking of the polymer matrix of the hydrogel.

Preferably, the polymers are covalently crosslinked.

More preferably, the crosslinking of the polymer is made in presence of a crosslinking agent chosen from dithiol molecules such as for example dithioerythriol.

The patterning of the hydrogel is typically performed by standard photolithographic techniques or with a direct LASER writing equipment.

These techniques are notably disclosed in Chollet, B., D'Eramo, L., Martwong, E., Li, M., Macron, J., Mai, T. Q., Tabeling, P. and Tran, Y., 2016. Tailoring patterns of surface-attached multiresponsive polymer networks. ACS applied materials & interfaces, 8(37), pp.24870-24879.

The grafting of the nucleic acids is typically performed by spotting or in-situ light directed synthesis, respectively detailed in DeRisi, J. et al. Use of a cDNA microarray to analyse gene expression. Nat. Genet 14, 457-460 (1996) and in Fodor, S. P. et al. Light-directed, spatially addressable parallel chemical synthesis. Science (80-.). 251, 767-773 (1991).

Advantageously, during step 5), the first and the second substrates are positioned in a way permitting the nucleic acids to be inside the cages, when the hydrogel is in the swollen state.

More advantageously, the nucleic acids are grafted either on the first substrate, inside the closed patterns or on the second substrate, opposite the closed patterns.

The bonding step may be performed according to any known process.

According to a first embodiment, the bonding step is performed by oxygen plasma treatment. Preferably, the oxygen plasma treatment is made at room temperature, typically at a temperature ranging from 5 to 50° C., more preferably from 10 to 40° C., even more preferably from 15 to 30° C. Preferably, the duration of the oxygen plasma treatment ranges from 10 s to 2 min, more preferably from 30 s to 1 min.

Preferably, according to this first embodiment, the process further comprises, before step 6), a preparation step of deposition on the nucleic acids of a mask capable of protecting the nucleic acids during exposure to the oxygen plasma. The mask is typically made of an adhesive tape, preferably made of a material chosen from plastic film, paper, cloth, foam or foil coated with an adhesive. The mask is finally removed after the plasma treatment, typically by peeling.

According to a second embodiment, the bonding step is performed by the application of a pressure on the surface of the device. Preferably, according to this embodiment, the pressure on the surface of the device is performed by applying a negative pressure into an external microfluidic channel surrounding the main design.

According to third embodiment, the bonding step is performed by using of a crosslinkable composition comprising at least one polymer and optionally at least one crosslinking agent. According to this third embodiment, the bonding step is performed as follows:
a) bringing together the first and the second walls,
b) depositing between the two walls a layer of a composition comprising at least one polymer and at least one crosslinking agent in order to fill the gap between the first wall and the second wall, and
c) crosslinking, preferably self-crosslinking, of the at least one polymer.

Preferably, the polymer is chosen among polyepoxides

The process may further comprise:
between steps 1) and 2), an intermediary step of structuring and/or functionalizing the surface of the first substrate, and/or
between steps 3) and 4), an intermediary step of structuring and/or functionalizing the surface of the second substrate.

When the substrate is made of a hydrogel, the functionalization of the substrate may typically be performed by following the protocol described in Chollet, B. D'eramo, L., Martwong, E., Li, M., Macron, J., Mai, T. Q., Tabeling, P. and Tran, Y., 2016. Tailoring patterns of surface-attached multiresponsive polymer networks. ACS applied materials & interfaces, 8(37), pp.24870-24879.

If the structures are not made of hydrogel, then functionalization may typically be performed following the protocol detailed in Beal, John H L et al. “A rapid, inexpensive surface treatment for enhanced functionality of polydimethylsiloxane microfluidic channels.” Biomicrofluidics vol. 6,3 36503. 30 Jul. 2012

When the substrate is made of a hydrogel, the structuration of the substrate may typically be performed by following the protocol described in Chollet, B. D'eramo, L., Martwong, E., Li, M., Macron, J., Mai, T. Q., Tabeling, P. and Tran, Y., 2016. Tailoring patterns of surface-attached multiresponsive polymer networks. ACS applied materials & interfaces, 8(37), pp.24870-24879.

When the substrate is made of a non-swellable material, the structuration of the substrate may typically be performed following standard lithography protocols, notably standard photolithography protocols.

According to a particular embodiment, the process may further comprise, before the deposition the hydrogel material, an additional step consisting of the deposition on the surface of the substrate of a nanoparticle layer, preferably a patterned chromium/gold bilayer.

The deposition of the patterned layer may for example be performed by standard photolithography.

According to a particular embodiment, the method further comprises at least one of the following steps:
a) grafting (directly) a plurality of ligands on the surface of the first substrate (14) and/or on the surface of the second substrate (20), and/or
b) grafting (indirectly) a plurality of ligands on the surface of the first substrate (14) and/or on the surface of the second substrate (20).

Step a) defined above may be performed at any time of the manufacturing method defined above. In particular, step a) may be performed before or after the grafting of the closed patterns (16), before or after the grafting of the nucleic acids (22).

According to a first embodiment, the indirect grafting of ligands is performed by associating to the plurality of grafted nucleic acids (22) a plurality of ligands by hybridization, said plurality of ligands being conjugated with a nucleic acid having complementarity with at least a part of the grafted nucleic acids (22).

According to this first embodiment, step b) is preferably performed after the grafting of the nucleic acids (22). Step b) can be performed until the conditions are modified to actuate the hydrogel into swollen state, thereby trapping cells or organelles in a cage formed by the first (14) and second (20) walls of the microfluidic device (10), and the closed pattern (16) of hydrogel in swollen state.

According to a second embodiment, the indirect grafting of the ligands comprises i) coating an adhesion coating on at least part of the surface of the first (14) and/or second (20) substrate, and ii) grafting the ligands to said adhesion coating.

Step i) can be performed before or after the grafting of the closed patterns (16), before or after the grafting of the nucleic acids (22).

Step ii) is preferably performed after the deposition of the adhesion coating. Step ii) can be performed until the conditions are modified to actuate the hydrogel into swollen state, thereby trapping cells or organelles in a cage formed by the first (14) and second (20) walls of the microfluidic device (10), and the closed pattern (16) of hydrogel in swollen state.

The method for the manufacture of a microfluidic device further comprises one or more of the following steps:
1) Hybridizing a DNA comprising a sequence complementary to all or part of a constant sequence present in grafted nucleic acids, in particular present in all or part of the grafted nucleic acids ; in particular, one or a plurality of different DNA comprising a sequence complementary to all or part of the constant sequence may be used;
2) Extending the hybridizing DNA by polymerisation (e.g. using Maxima, SuperScript RT, Phusion or Q5 polymerase);
3) Ligating the grafted nucleic acids, in particular grafted DNA, with a or another DNA sequence; and/or
4) Releasing all or part of the grafted nucleic acid, possibly previously modified by hybridization, extension or ligation according to 1), 2) or 3), from the surface of the first or second substrate, by cleavage (for instance by photocleavage or cleavage catalyzed by an endonuclease).

In some embodiments, the hybridizing DNA forms together with the grafted nucleic acid, a double stranded DNA containing a restriction site for an endonuclease.

The process may also comprise a further step of fixing structures capable of capturing a cell or an organelle.

This supplemental step is typically realized by standard photolithography.

The microfluidic device of the invention can be used in methods of sequencing cells or cell organelles, with the possibility of combining phenotypic information from optical imaging and -omics information for a single cell or organelle, or for e.g. two or more cells in interaction, and this for thousands of cells simultaneously.

The method of performing analysis of cell or organelles comprises:
a) Providing the microfluidic device and a preparation of cells or organelles labelled with a nucleic acid comprising a localisation sequence as obtainable by the method of the invention;
b) Optionally, associating all or part of the cells or organelles labelled with the nucleic acid comprising the localisation sequence in a compartment with a common labeling nucleic acid sequence or with a plurality of different labeling nucleic acid sequences;
c) Injecting in the microfluidic device the cells or organelles labelled with the nucleic acid comprising the localisation sequence in suspension under conditions in which the hydrogel is in retracted state;
d) Modifying the conditions to actuate the hydrogel into swollen state, thereby trapping cells or organelles in a cage formed by the first and second walls of the microfluidic device, and the closed pattern of hydrogel in swollen state;
e) Optionally analyzing captured cells or organelles and/or molecules they secrete, using optical imaging;
f) Optionally releasing in the cage, the grafted nucleic acids from the surface of the first or second substrate of the microfluidic device;
g) Optionally, lysing trapped cells or organelles, thereby releasing cellular or organellar nucleic acids in the cages;
h) Associating the barcode of the nucleic acids with either the released cellular or organellar nucleic acids and/or labeling nucleic acid sequence(s) thereby forming barcoded nucleic acids;
i) Modifying the conditions to actuate the hydrogel into the retracted state;
j) Releasing the grafted nucleic acids from the first or second substrate of the microfluidic device, if not released in f);
k) Recovering and sequencing the barcoded nucleic acids; and
l) Optionally mapping the barcoded sequencing data onto the data from optical imaging obtained in e).

At step c), injecting in the microfluidic device the cells or organelles labelled with the nucleic acid comprising the localisation sequence in suspension under conditions in which the hydrogel is in retracted state is typically performed by setting the temperature, pressure or pH—depending on the nature of the actuatable hydrogel—so that the hydrogel is in retracted state. For instance, if the microfluidic device comprises a lower critical solution temperature (LCST) temperature-responsive hydrogel, the temperature of the microfluidic device is raised above the lower critical solution temperature (LCST) to retract the hydrogel. For a temperature-responsive hydrogel comprising or consisting of poly(N-isopropylacrylamide (PNIPAM), the hydrogel is fully expanded at ≤28° C., fully retracted at ≥36° C., and partially expanded between these temperatures, allowing cages to be fully open at 37° C. for cell or organelle loading (D'Eramo et al., Microsystems & Nanoengineering (2018) 4, 17069). For instance, if the microfluidic device comprises an upper critical solution temperature (UCST) temperature-responsive hydrogel, the temperature of the microfluidic device is decreased below the upper critical solution temperature (UCST) to retract the hydrogel. For a temperature-responsive hydrogel comprising or consisting of P(MA-AM-AMA) the hydrogel is fully retracted at ≤10° C., fully expanded at ≥50° C., and partially expanded between these temperatures, allowing cages to be fully open at 10° C. for cell or organelle loading. According to an embodiment, at step d), single cells or single organelles are trapped in the cages. According to another embodiment two (or more) interacting cells are trapped in the cages, for example plasma cell and reporter cell; cytotoxic T cell (or CAR T cell) and target cell (e.g. tumor cell); T cell and antigen-presenting cell.

To actuate the hydrogel into swollen state, the temperature, pressure or pH—depending on the nature of the actuatable hydrogel—is modified so that the hydrogel swells and comes into contact with the second substrate. For instance, if the microfluidic device comprises a lower critical solution temperature (LCST) temperature-responsive hydrogel, the temperature of the microfluidic device is reduced below the lower critical solution temperature (LCST) to swell the hydrogel. For a temperature-responsive hydrogel comprising or consisting of poly(N-isopropylacrylamide) (PNIPAM), the temperature can typically be set at ≤28° C., where the hydrogel is fully expanded (D'Eramo et al., Microsystems & Nanoengineering (2018) 4, 17069). For instance, if the microfluidic device comprises an upper critical solution temperature (UCST) temperature-responsive hydrogel, the temperature of the microfluidic device is raised above the upper critical solution temperature (UCST) to expand the hydrogel. For a temperature-responsive hydrogel comprising or consisting of P(MA-AM-AMA) the hydrogel is fully expanded at ≥50° C.

The method may further comprise, between steps d) and h), changing surrounding conditions of the cells or organelles. Changing surrounding conditions includes circulating in the microfluidic device an aqueous phase containing, e.g. salts, detergents, proteins, and/or nucleic acid sequences. Changing surrounding conditions includes exchanging molecules, such as salts, that pass through the hydrogel of closed cages, by fully opening cages in the case that cages also comprise structures capable of capturing a cell or an organelle, or partially opening the cages.

According to an embodiment, the method further comprises, for example after step e) and before step f), but not necessarily:
e1) Binding of an analyte or of analytes secreted or released by the captured cells or organelles to ligands grafted directly or indirectly to the surface of the first substrate (14) and/or on the surface of the second substrate (20);
e2) Detecting the analyte or analytes bound to the grafted ligand by binding with a labeled second ligand or labeled ligands that is/are specific for the bound analyte or analytes.

According to a first embodiment, at step e2, detecting is performed directly with a second ligand or ligands fluorescently labeled.

According to a second embodiment, at step e2, detecting is performed indirectly, with a second ligand or ligands labeled with a ligand identification nucleic acid to the analyte or analytes bound to the grafted ligand, wherein the sequence of said ligand identification nucleic acid allows identification of the ligand and the analyte or analytes bound to the grafted ligand.

According to this second embodiment, the method may further comprise amplifying the sequence of said ligand identification nucleic acid. Amplification preferably consists of a linear amplification, more preferably by using at least one polymerase and at least one restriction or nicking enzyme.

According to this second embodiment of the method, in step h), the method may further comprise associating the barcode of the nucleic acids (22) with the ligand identification nucleic acid, thereby forming barcoded nucleic acids.

According to an embodiment, the common labeling DNA sequence or plurality of different labeling DNA sequences provided at step b) are used for DNA-toolbox reactions (or dynamic DNA reaction network) for phenotype sorting of cells or organelles, thereby actuating the release of the grafted nucleic acids in step f) or j). The principles of DNA-toolbox reactions are described for instance in the international patent applications WO2017141068 and WO2017141067.

According to an embodiment, at step g), trapped cells or organelles are lysed by osmotic shock. This may be readily implemented by the skilled person by circulating in the microfluidic device a hypo- or hyper-osmotic aqueous phase. The cages may be retained closed for this operation.

According to an embodiment, step h) comprises hybridizing the nucleic acids comprising barcodes, which may be still grafted the surface of the first or second substrate of the microfluidic device or released from the surface of the first or second substrate of the microfluidic device, by complementarity to the released cellular or organellar nucleic acids and/or to labeling nucleic acid sequence(s). In particular, where the nucleic acids comprising a barcode are DNA, step h) [or the method between steps i) and j)] may additionally comprise extending the DNA comprising a barcode hybridized to the released cellular or organellar nucleic acids (or labeling nucleic acid sequence(s)) using a DNA polymerase to create the complementary strand of the released cellular or organellar nucleic acids (or labeling nucleic acid sequence(s)) which comprises a barcode. The nucleic acids may comprise, for example, a 3′-region of sequence oligo d(T) or oligo d(T)VN, for hybridization to the poly(A) tail of mRNA (for mRNA sequencing), a 3′-region of sequence complementary to that of a specific RNA (for targeted RNA sequencing) or DNA (for targeted DNA sequencing), a 3′-region of random sequence, for example d(N)6 (for RNA sequencing or DNA sequencing), a 3′-region with three ribo(G) nucleotides for reverse transcriptase template switching (for RNA sequencing), or a 3′-region complementary to a nucleotide sequence introduced by recombination, for example after “tagmentation” catalyzed by Tn5 transposase. The latter can be used, for example, for genomic DNA sequencing, or epigenomic analysis of DNA methylation (using Methyl-seq or bisulfite sequencing) or chromatin structure (using transposase-accessible chromatin with sequencing, ATAC-Seq), or for RNA sequencing after tagmentation of RNA-DNA duplexes formed after first strand cDNA synthesis or double-stranded DNA formed after first and second strand cDNA synthesis on RNA released by the cells or organelles.

According to another embodiment, the nucleic acids comprising a barcode are DNA, and may be wholly or partly double-stranded, and step h) comprises ligating the DNA comprising a barcode to DNA released by the cells or organelles. For example, the barcodes may be ligated to genomic DNA, for example after restriction digestion (for genomic DNA sequencing or analysis of DNA methylation), or after digestion with micrococcal nuclease (for metagenomic analysis using MNase-seq or ChIP-seq).

According to still another embodiment, the nucleic acids comprising a barcode are DNA, and may be wholly or partly double-stranded, and step h) comprises recombining the DNA comprising barcode with DNA released by the cells or organelles. For example, the barcodes may be recombined with genomic DNA for genomic DNA sequencing, or epigenomic analysis of DNA methylation (using Methyl-seq or bisulfite sequencing) or chromatin structure (using transposase-accessible chromatin with sequencing, ATAC-Seq). Alternatively, the nucleic acids comprising a barcode recombine with RNA-DNA duplexes formed after first strand cDNA synthesis on RNA released by the cells or organelles, or recombining with double-stranded DNA formed after first and second strand cDNA synthesis on RNA released by the cells or organelles (for RNA sequencing). In a preferred embodiment the oligonucleotide comprises a Mosaic End (ME) sequence which recombines with DNA catalyzed by Tn5 transposase.

According to an embodiment, the method further comprises between steps d) and h), releasing a the nucleic acids comprising barcodes upon the presence of a cellular or organellar material (e.g. a surface molecule, a secreted molecule, or a lysis product) in the cages, e.g. by a proximity ligation assay, or proximity extension assay

### Kits for Mapping and Sequencing Individual Cells or Organelles

The invention further relates to a kit for implementing the above method of mapping and sequencing which comprises the constituent of the kit for labelling individual cells or organelles as defined above and a compartment as defined above.

The invention is further exemplified by the following figures.

### FIGURES

FIG. 1: Example of single cell mapping-sequencing (scMap-seq) procedure combining microcontact printing of the tissue to be analyzed coupled to single cell analysis by in a microfluidic device. A DNA chip comprising position-specific biotinylated sequences is transferred (optionally via a hydrogel buffer) to a section of tissue coated with streptavidin ligands by microcontact printing. This generates a tissue with different marked areas. The marked tissue is then dissociated. The labelled and dissociated cells are transferred to a single cell analysis system by hydrogel cages.

FIG. 2: Example of single stranded biotinylated localisation nucleic acid with 3′ anchoring for spatial mapping, and scRNA-seq/CITE-seq like experiment, comprising from the 5′ end to the 3′ end: a first constant sequence (‘PCR primer’), a localisation sequence (‘barcode’), a second constant sequence (‘Capture primer’), and a Spacer sequence bound by its 3′ end to the substrate of the nucleic acid array. The localisation nucleic acid is partly released from the nucleic acid array by hybridizing a second nucleic acid (‘Bmtl’) to the 3′-second constant sequence, thereby creating a restriction target site, and cleaving with said restriction endonuclease. After transfer, the released biotinylated oligonucleotide binds via streptavidin ligands to the tissue surface. The Capture primer is designed to hybridize to oligonucleotides of a single cell analysis system, wherein some of the mRNA-specific primer sequences (poly-dT(V)N hybridizing to poly(A) mRNA) are replaced by a sequence complementary to the Capture primer.

FIG. 3: Other example of single stranded biotinylated localisation nucleic acid with 3′ anchoring for spatial mapping, and scRNA-seq/CITE-seq like experiment, wherein the .anchored nucleic acid is biotinylated at its 5′ end.

FIG. 4: Example of single stranded biotinylated localisation nucleic acid with 3′ anchoring for spatial mapping, and scRNA-seq/CITE-seq like experiment, involving elongation. The localisation nucleic acid is partly released from the nucleic acid array by hybridizing a second nucleic acid (‘Bmtl’) to the 3′-second constant sequence, thereby creating a restriction target site, and cleaving with said restriction endonuclease. After transfer, the released localisation nucleic acid hybridises to a capture nucleic acid which is attached to the ligand bound to a receptor of cells of the tissue sample. The capture nucleic acid comprises a sequence complementary to the first constant sequence (‘PCR primer’) of the localisation nucleic acid, and the 3′ end of the strand of the capture nucleic acid is elongated by polymerisation. 5′ mononucleotides are then removed from the extended double-stranded nucleic acid, using an exonuclease acting in the 5′ to 3′ direction.

FIG. 5: Example of single stranded biotinylated localisation nucleic acid with 3′ anchoring for spatial mapping, and scRNA-seq/CITE-seq like experiment, involving elongation. A part of localisation nucleic acid (“PCR primer”) is hybridised to the capture nucleic acid (“Nicking Site”), and the 3′ end of capture nucleic acid is extended by polymerisation, thereby synthesising a sequence complementary to said localisation nucleic acid. The hybridised capture nucleic acid forms, together with the first (3′) constant sequence of the localisation nucleic acid, a stretch of double stranded DNA containing a first restriction site, which is a nicking site for a nicking endonuclease. The nicking endonuclease is used to release, from the substrate of the nucleic acid array, a part of the localisation nucleic acid hybridised to the capture nucleic acid bound to the ligand or member of the non-covalent interacting pair. 5′ mononucleotides are then removed from the extended double-stranded nucleic acid, using an exonuclease acting in the 5′ to 3′ direction.

FIG. 6: Example of single stranded biotinylated localisation nucleic acid with 3′ anchoring for spatial mapping, and scRNA-seq/CITE-seq like experiment, involving elongation. The localisation nucleic acid is partly released from the nucleic acid array by hybridizing a second nucleic acid (‘Bmtl’) to the 3′-second constant sequence, thereby creating a restriction target site, and cleaving with said restriction endonuclease. A template-dependent DNA polymerase (e.g. Phusion) is used to synthesise a strand complementary to the released localisation nucleic acids, extending the second nucleic acid (‘Bmtl’), thereby forming partly or fully doubled stranded nucleic acids. Partly 5′ mononucleotides are removed using an exonuclease acting in the 5′ to 3′ direction (e.g. T7 exonuclease) thereby producing partly doubled stranded localisation nucleic acids with stick ends, before ligating the localisation nucleic acids with the capture nucleic acids.

FIG. 7: Example of single stranded biotinylated localisation nucleic acid with 3′ anchoring for spatial mapping, and scRNA-seq/CITE-seq like experiment where the third nucleic acid forms, together with the second constant sequence, a stretch of double stranded DNA containing a second restriction site for a second endonuclease, and the double stranded DNA when cleaved by the second endonuclease forms a ligation site. For binding the released localisation nucleic acids to cells of the tissue sample via the ligand bound to a receptor or receptors of cells or organelles, a ligand is used which is covalently or non-covalently attached to a partly doubled stranded capture nucleic acid, wherein the protruding strand is complementary to the non-hybridised region of the second constant sequence. A ligase repair the nick between partly doubled stranded capture nucleic acid and the partly doubled stranded formed by the third nucleic acid is hybridised to the second constant sequence of the released localisation nucleic acids

FIG. 8: Example of single stranded biotinylated localisation nucleic acid with 3′ anchoring for spatial mapping, and scRNA-seq/CITE-seq like experiment where the capture nucleic acid forms, together with the second constant sequence, a stretch of double stranded DNA containing a restriction site for an endonuclease, and the double stranded DNA when cleaved by the endonuclease releases all or part of the localisation nucleic acid.

FIG. 9: Example of single stranded biotinylated localisation nucleic acid with 3′ anchoring for spatial mapping, and scRNA-seq/CITE-seq like experiment. The third nucleic acid (“nick”) forms, together with the second constant sequence (“nick”), a stretch of double stranded DNA containing a restriction site for an endonuclease which is a nicking endonuclease. A strand displacing, template-dependent DNA polymerase (e.g. phi29) synthesise a strand complementary to at least part of the second (3′) constant sequence (“PCR primer”), the localisation sequence (“Sp. barcode”), and the first (5′) constant sequence (“Capture Seq.”). The nucleic acid complementary to the localisation nucleic acid are released by cleaving with the nicking endonuclease and strand-displacement by the template-dependent DNA polymerase that primes at the nick. The released complementary nucleic acid hybridized to a capture nucleic acid, covalently or non-covalently bound to the ligand, which comprises at least part of the second constant sequence (“PCR Primer”).

FIG. 10: Example of double stranded biotinylated localisation nucleic acid with 3′ anchoring for spatial mapping, and scATAC-seq/scCHIP-seq like experiment. The 3′ end of the hybridized third nucleic acid is extended using a non-strand displacing, template-dependent DNA polymerase and the nick between the extended third nucleic acid and the second nucleic acid is repaired by a ligase, thereby creating a fourth nucleic acid comprising, from the 5′ to 3′ end, the sequence of the third nucleic acid, the sequence complementary to that of the localization sequence, and the sequence of the second nucleic acid.

FIG. 11: Example of single stranded localisation nucleic acid with 3′ anchoring for spatial mapping, and scRNA-seq/CITE-seq like experiment wherein the second nucleic acid is attached, covalently or non-covalently to the ligand which binds to a receptor or receptors of the cells or organelles in the tissue sample.

FIG. 12: Example of single stranded biotinylated localisation nucleic acid with 5′ anchoring for spatial mapping, and scRNA-seq/CITE-seq like experiment wherein the second nucleic acid is covalently bound at its 3′ end to a biotin, while the ligand binds to a streptavidin. The hybridized second nucleic acid forms, together with the first (5′) constant sequence, a stretch of double stranded DNA containing a first restriction site, which is a nicking site for a nicking endonuclease. The nicking endonuclease releases, from the substrate of the nucleic acid array, a part of the localisation nucleic acid hybridised to the second nucleic acid bound to the biotin.

FIG. 13: Example of single stranded biotinylated localisation nucleic acid with 5′ anchoring for spatial mapping, and scRNA-seq/CITE-seq like experiment. A ligand is used which is covalently or non-covalently attached to a capture nucleic acid comprising a region fully or partly complementary to the second (3′) constant sequence. The released localisation nucleic acid comprises i) all or part of the first (5′) constant sequence, ii) the localisation sequence, and iii) the second (3′) constant sequence hybridised to the complementary region of the capture nucleic acid. The 3′ end of the second (3′) constant sequence and the 3′ end of the complementary region of the capture nucleic acid is extended, using a template-dependent DNA polymerase (e.g. phi29), thereby forming an extended double-stranded nucleic acid. The 5′ mononucleotides are removed from the extended double-stranded nucleic acid, using an exonuclease acting in the 5′ to 3′ direction (e.g. T7 exonuclease).

FIG. 14: Example of double stranded biotinylated localisation nucleic acid with 5′ anchoring for spatial mapping, and scATAC-seq/scCHIP-seq like experiment, involving hybridising the localisation nucleic acid and capture nucleic acid, and extending the 3′ end of capture nucleic acid by polymerisation, thereby synthesising a sequence complementary to said localisation nucleic acid. A stretch of double stranded DNA containing a restriction site for a second endonuclease is formed, allowing the release of the localisation nucleic acids. 5′ mononucleotides are then removed from the extended double-stranded nucleic acid, using an exonuclease acting in the 5′ to 3′ direction.

FIG. 15: Example of single stranded biotinylated localisation nucleic acid with 5′ anchoring for spatial mapping, and scRNA-seq/CITE-seq like experiment. A ligand is used which is covalently or non-covalently attached to a capture nucleic acid comprising a strand complementary to part of the first (5′) constant sequence. The released localisation nucleic acids comprising i) all or part of the first (5′) constant sequence, ii) the localisation sequence, and iii) the second (3′) constant sequence hybridise to the complementary region of the capture nucleic acid. Using a template-dependant non-strand displacing DNA polymerase (e.g. Phusion) a strand complementary to the capture nucleic acids is synthetised and the localisation nucleic acids is ligated with the capture nucleic acids.

FIG. 16: Example of single stranded biotinylated localisation nucleic acid with 5′ anchoring for spatial mapping, and scRNA-seq/CITE-seq like experiment. The first endonuclease is used to release all or part of the localisation nucleic acids from the substrate of the nucleic acid array, wherein released localisation nucleic acid comprises i) all or part of the first (5′) constant sequence, ii) the localisation sequence, and iii) the second (3′) constant sequence. For binding the released localisation nucleic acids to cells of the tissue sample via the ligand bound to a receptor or receptors of cells, a ligand is used which is covalently or non-covalently attached to a partly double stranded capture nucleic acid, wherein the protruding strand is complementary to 3′ end of the second constant sequence. A ligase is used to repair the nick between partly double stranded capture nucleic acid and the partly doubled stranded nucleic acid formed by the protruding strand of the capture nucleic acid hybridised to the second constant sequence of the released localisation nucleic acids.

FIG. 17: Example of double stranded biotinylated localisation nucleic acid with 5′ anchoring for spatial mapping, and scATAC-seq/scCHIP-seq like experiment, wherein the second nucleic acid is covalently bound at its 5′ end to a biotin and hybridized to the 3′-second constant sequence, while the ligand binds to a streptavidin. The localisation nucleic acid is partly released from the nucleic acid array by hybridizing a third nucleic acid (‘Nhel) to the 5′-second constant sequence, thereby creating a restriction target site, and cleaving with said restriction endonuclease. The released localisation nucleic acid comprises i) all or part of the first (5′) constant sequence (“Capture primer”), ii) the localisation sequence (“Sp. Barcode”), and iii) the second (3′) constant sequence (“PCR primer”. The 3′ end of the second (3′) constant sequence and the 3′ end of the complementary region of the capture nucleic acid is extended, using a template-dependent DNA polymerase (e.g. phi29), thereby forming an extended double-stranded nucleic acid.

FIG. 18: Example of double stranded biotinylated localisation nucleic acid with 5′ anchoring for spatial mapping, and scATAC-seq/scCHIP-seq like experiment. A ligand is used which is attached to a capture nucleic acid comprising a region fully or partly complementary to the second (3′) constant sequence. The localisation nucleic acid is partly released from the nucleic acid array by hybridizing a second nucleic acid (‘Nhel) to the 5′-second constant sequence, thereby creating a restriction target site, and cleaving with said restriction endonuclease. The released localisation nucleic acid comprises i) all or part of the first (5′) constant sequence (“Capture primer”), ii) the localisation sequence (“Sp. Barcode”), and iii) the second (3′) constant sequence (“PCR primer”) hybridised to the complementary region of the capture nucleic acid (“PCR primer”). The 3′ end of the second (3′) constant sequence and the 3′ end of the complementary region of the capture nucleic acid is extended, using a template-dependent DNA polymerase (e.g. phi29), thereby forming an extended double-stranded nucleic acid.

FIG. 19: Example of double stranded biotinylated localisation nucleic acid with 5′ anchoring for spatial mapping, and scATAC-seq/scCHIP-seq like experiment. The first endonuclease is used to release all or part of the localisation nucleic acids from the substrate of the nucleic acid array, wherein released localisation nucleic acid comprises i) all or part of the first (5′) constant sequence (“PCR primer”), ii) the localisation sequence (“Sp. barcode”), and iii) the second (3′) constant sequence (“Ligation site”). For binding the released localisation nucleic acids to cells of the tissue sample via the ligand bound to a receptor or receptors of cells, a ligand is used which is covalently or non-covalently attached to a double stranded capture nucleic acid, wherein the protruding strand is complementary to 3′ end of the second constant sequence. A ligase is used to repair the nick between partly double stranded capture nucleic acid and the partly doubled stranded nucleic acid formed by the protruding strand of the capture nucleic acid hybridised to the second constant sequence of the released localisation nucleic acids and the 3′ end of the complementary region of the second (3′) constant sequence (“Ligation site”) is extended, using a template-dependent DNA polymerase (e.g. phi29), thereby forming an extended double-stranded nucleic acid.

FIG. 20: Example of double stranded biotinylated localisation nucleic acid with 5′ anchoring for spatial mapping, and scATAC-seq/scCHIP-seq like experiment. The first endonuclease is used to release all or part of the localisation nucleic acids from the substrate of the nucleic acid array, wherein released localisation nucleic acid comprises i) all or part of the first (5′) constant sequence (“Capture seq.”), ii) the localisation sequence (“Sp. barcode”), and iii) the second (3′) constant sequence (“Ligation site”). For binding the released localisation nucleic acids to cells of the tissue sample via the ligand bound to a receptor or receptors of cells, a ligand is used which is covalently or non-covalently attached to a double stranded capture nucleic acid, wherein the protruding strand is complementary to 3′ end of the second constant sequence. A ligase is used to repair the nick between partly double stranded capture nucleic acid and the partly doubled stranded nucleic acid formed by the protruding strand of the capture nucleic acid hybridised to the second constant sequence of the released localisation nucleic acids and the 3′ end of the complementary region of the second (3′) constant sequence (“Ligation site”) is extended, using a template-dependent DNA polymerase (e.g. phi29), thereby forming an extended double-stranded nucleic acid.

FIG. 21: Example of single stranded biotinylated localisation nucleic acid with 5′ anchoring for spatial mapping, and scRNA-seq/CITE-seq like experiment wherein the second nucleic acid is covalently bound at its 3′ end to a ligand bound to a receptor or receptors of cells. The hybridized second nucleic acid forms, together with the first (5′) constant sequence, a stretch of double stranded DNA containing a first restriction site, which is a nicking site for a nicking endonuclease. The nicking endonuclease releases, from the substrate of the nucleic acid array, a part

FIG. 22: DNA micro-array structure. a. general structure of a section of the micro array. The arrow indicates the position of a DNA strand. Each DNA spot is made up of a set of DNA strands b. 5′ to 3′ DNA construction of a strand of a specific spot. Strands from different sports carry different barcode sequences. Up to 1 million spot specific barcodes has been designed.

FIG. 23: DNA micro array structure for scCHIP-seq applications. a. General structure of a section of the micro array. The arrow indicates the position of a DNA strand. Each DNA spot is made up of a set of DNA strands b. 5′ to 3′ DNA construction of a strand of a specific spot. Strands from different spots carry different barcode sequences. Up to 1 million spot specific barcodes has been designed.

FIG. 24: DNA micro array structure for scATAC-seq applications. a. General structure of a section of the micro array. The arrow indicates the position of a DNA strand. Each DNA spot is made up of a set of DNA strands b. 5′ to 3′ DNA construction of a strand of a specific spot. Strands from different spots carry different barcode sequences. Up to 1 million spot specific barcodes has been designed.

FIG. 25: schematic representation of the closed patterns of a microfluidic device usable according to the invention. The microfluidic device 10 comprises a plurality of closed patterns 16 arranged in the form of a table. The closed patterns 16 represented are in the form of squares but may equivalently in the form of rectangles, circles or even hexagons. The closed patterns 16 have a thickness e and a height h.

FIG. 26: schematic representation of a microfluidic device wherein the patterns are made of an actuatable hydrogel. The microfluidic device 10 comprises a first wall 12 comprising a first substrate 14 on which a plurality of closed patterns 16 is grafted. A second wall 18, facing the first wall 12, comprises a second substrate 20. A plurality of nucleic acids 22 are grafted on the second substrate 20. The closed patterns 16 are made of an actuatable hydrogel which is swellable between a retracted state and a swollen state in which the closed patterns 16 and the second substrate 20 come into contact. The microfluidic device 10 further comprises an inlet 24 and an outlet 26 permitting respectively the introduction and the removal of reactants into the device 10.

On scheme A, the closed patterns 16 are in the retracted state. A gap 28 between the closed patterns 14 and the second substrate 20 allows fluids and cells present inside the device freely circulating inside the device 10.

Placed in specific physico-chemical conditions, the closed patterns 16 begin to absorb water and swell. The closed patterns 16 thus elongates until contacting the second substrate 20.

On scheme B, the closed patterns 16 are in the swollen state, in contact with the second substrate 20. The device 10 thus comprises a plurality of cages 30, each cage 30 being delimited by a lateral wall made of one of the closed patterns 16 and by end walls constituted of a portion of the first 14 and second 20 substrates.

FIG. 27: schematic representation of a microfluidic device wherein the second substrate is made of an actuatable hydrogel. The microfluidic device 10 comprises a first wall 12 comprising a first substrate 14 on which a plurality of closed patterns 16 is grafted. A second wall 18, facing the first wall 12, comprises a second substrate 20. A plurality of nucleic acids 22 are grafted on the first substrate 14. The second substrate 20 is made of an actuatable hydrogel which is swellable between a retraced state and a swollen state in which the closed patterns 16 and the second substrate 20 come into contact. The microfluidic device 10 further comprises an inlet 24 and an outlet 26 permitting respectively the introduction and the removal of reactants into the device 10.

On scheme A, the second substrate 20 is in the retraced state. A gap 28 between the closed patterns 14 and the second substrate 20 allows fluids and cells present inside the device freely circulating inside the device 10.

Placed in specific physico-chemical conditions, the second substrate 20 begins to absorb water and swells. The thickness of the second substrate 20 thus increases until contacting the closed patterns 16.

On scheme B, the second substrate 20 is in the swollen state, in contact with the closed patterns 16. The device 10 thus comprises a plurality of cages 30, each cage 30 being delimited by a lateral wall made of one of the closed patterns 16 and by end walls constituted of a portion of the first 14 and second 20 substrates.

FIG. 28 is a schematic representation of the main stages of the method of performing analysis of cells or organelles according to the invention. Stages are identified in ascending order from top to bottom.
Stage 1: Dissociated cells are injected into the microfluidic device at a temperature A, allowing the cages to be in the retracted state.

Stage 2: Cages are swollen by changing the temperature of the microfluidic device to temperature B allowing the capture of the cells. The cells are then lysed by changing the surrounding buffer, for example, using a low salt buffer. Untrapped cells are washed away.

Stage 3: Still at temperature B, the buffer is changed again to allow nucleic acid hybridization on the grafted oligonucleotides at the bottom of each cage.

Stage 4: Cages are retracted by changing the temperature of the microfluidic device to temperature A. This allows the injection and the incubation of a reaction mix in order to associate the barcode specific of each cage, present on the grafted oligonucleotides, to the hybridized nucleic acids released from the cell.

Stage 5: Still at temperature A, a new reaction mix is injected in order to release the grafted or hybridized barcoded cDNA and/or tags from the microfluidic device. The recovered sample is then purified, amplified and sequenced.

FIG. 29: Jurkat or Ramos cell line is labeled with fluorescent ubiquitous cell surface markers and mixed with unstained population for 30 min. Flow cytometry fluorescent measurements show separation between stained and unstained cells, also after mixing them, indicating an absence of cross-contamination after staining.

FIG. 30: Jurkat or Ramos cell line is labeled with ubiquitous cell surface markers carrying a fluorescent oligonucleotide and mixed for 30 min with a population stained with ubiquitous cell surface markers carrying a non-fluorescent oligonucelotide. Flow cytometry fluorescent measurements show separation between fluorescent and non-fluorescent cells, also after mixing them, indicating an absence of cross-talk after staining.

FIG. 31: Flow cytometry fluorescent measurement of unstained tissue (negative control), tissue stained before tissue dissociation (pre-dissociation staining) and tissue stained after tissue dissociation (post-dissociation staining). Bottom figures correspond to a labeling with ubiquitous and fluorescent cell surface markers. Top figures correspond to a labeling with ubiquitous cell surface markers carrying a fluorescent oligonucleotide (ASO: Antibody Streptavidin Oligonucleotide). We observe a separation between stained fluorescent and non-stained cells, indicating a resistance of the staining to the dissociation.

### EXAMPLES

### Example 1: Materials and Methods

The method for spatially resolving single-cell analysis comprises steps of tissue preparation (using fixed tissue or fresh tissue), DNA array preparation (using lyophilized or liquid enzyme), stamping (cut or cut and polymerization), tissue digestion (Collagenase I, Dnas I, Hyalurodinase), single-cell sequencing (in wells, droplets or actuatable cages) and analysis (with spatial reconstruction).

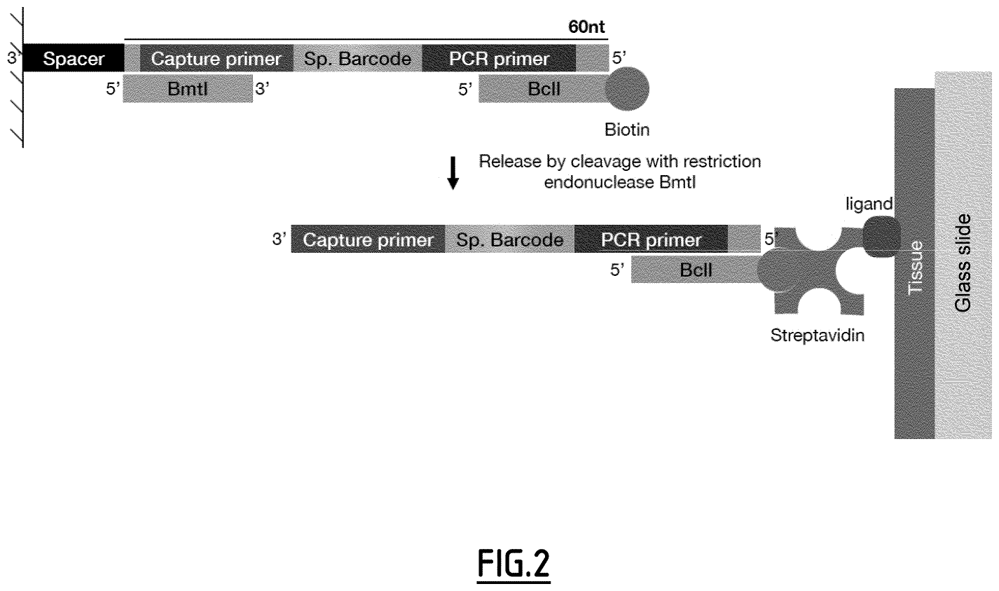

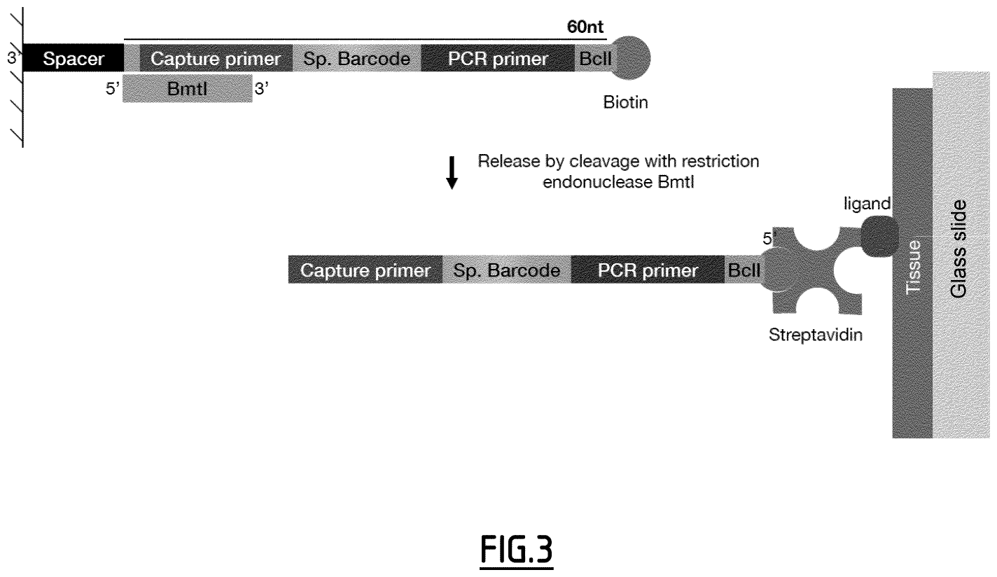

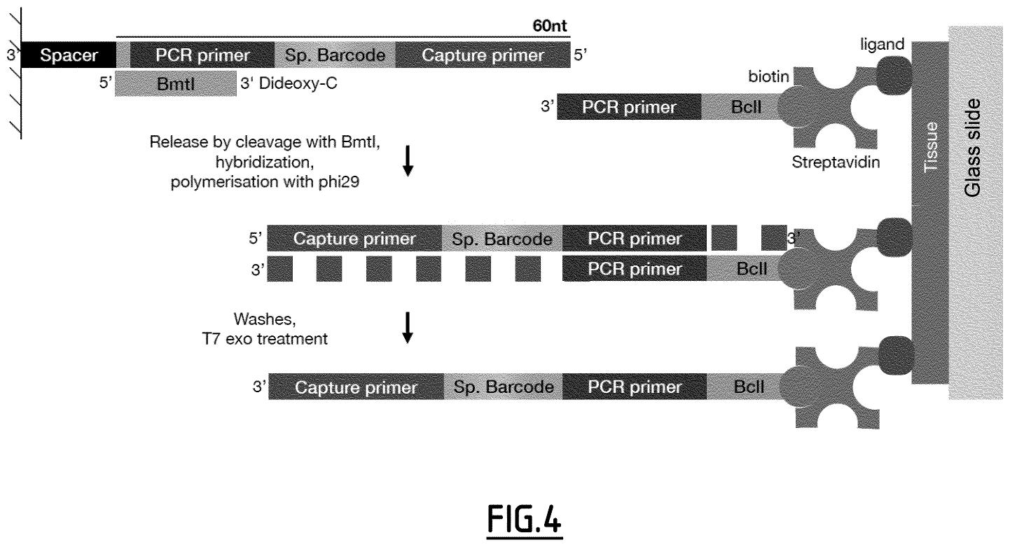

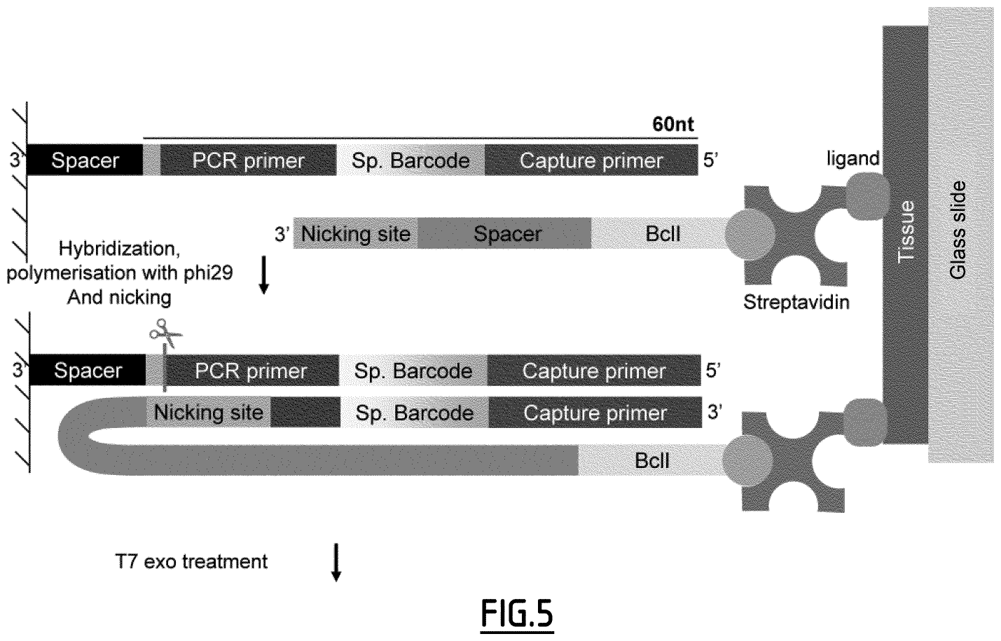

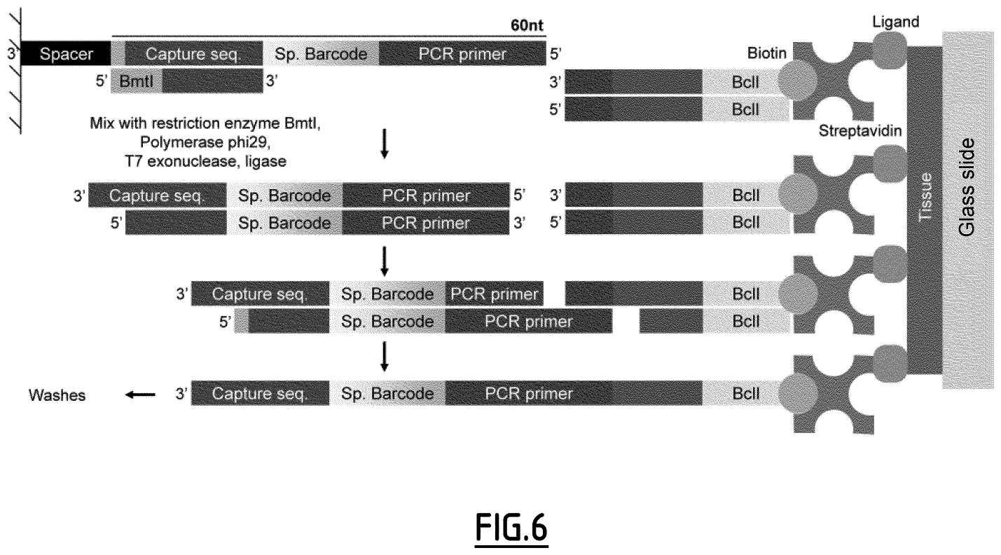

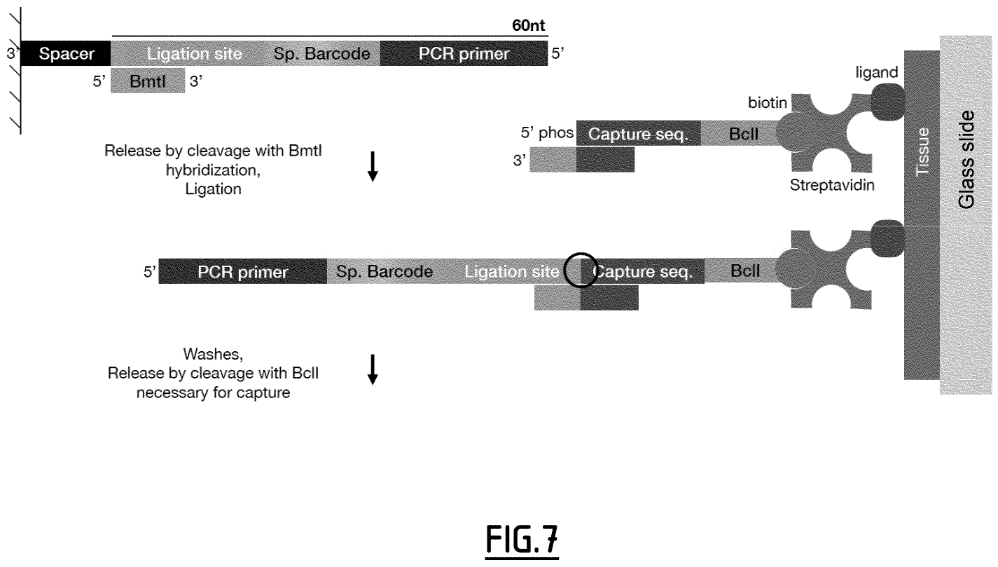

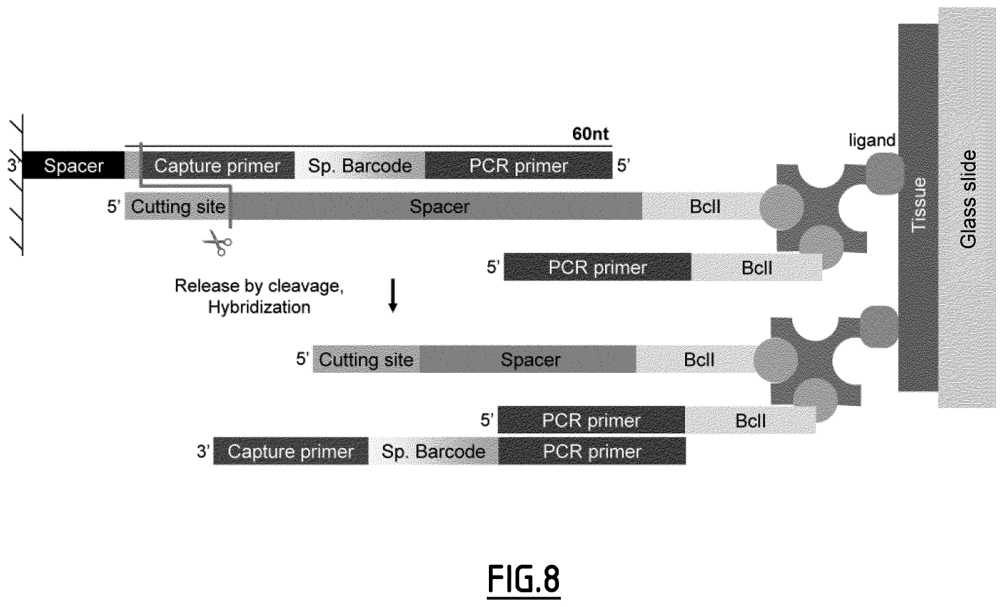

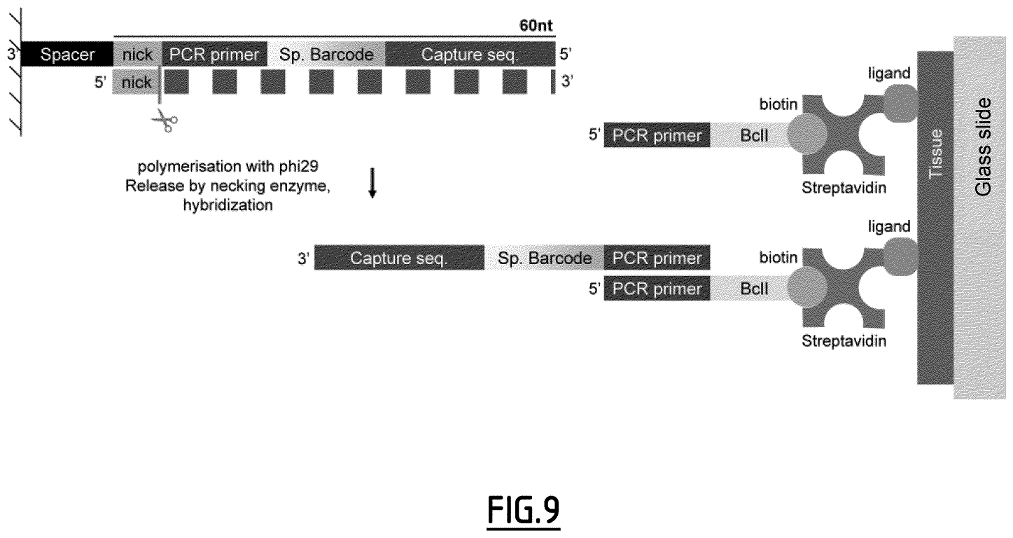

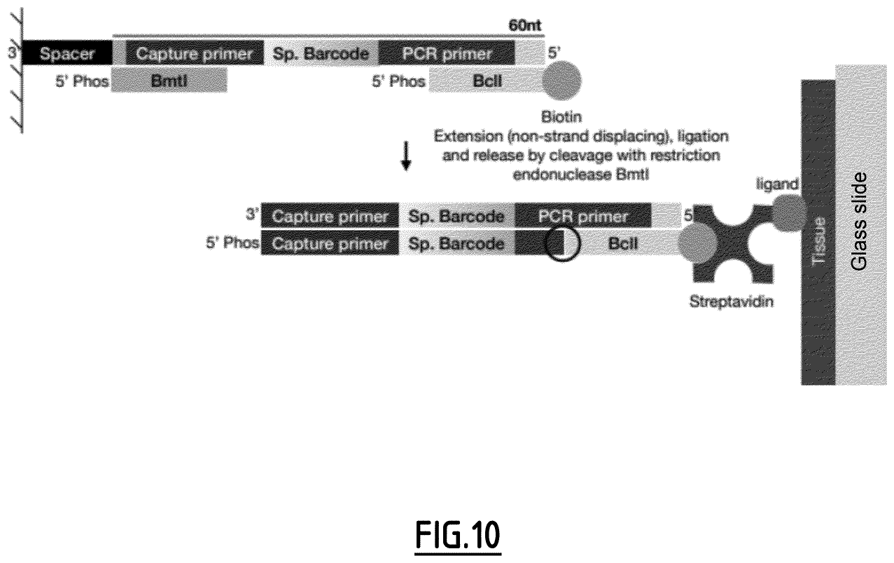

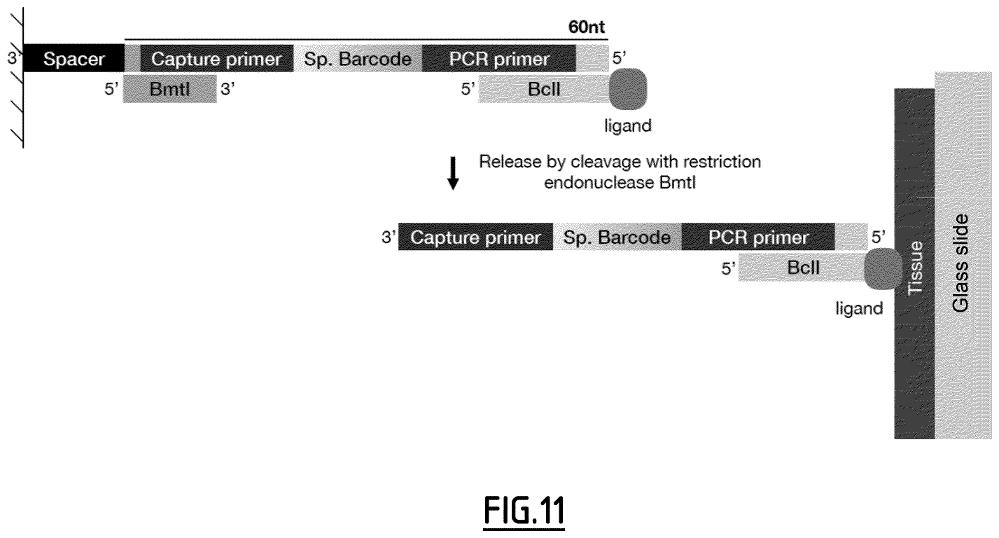

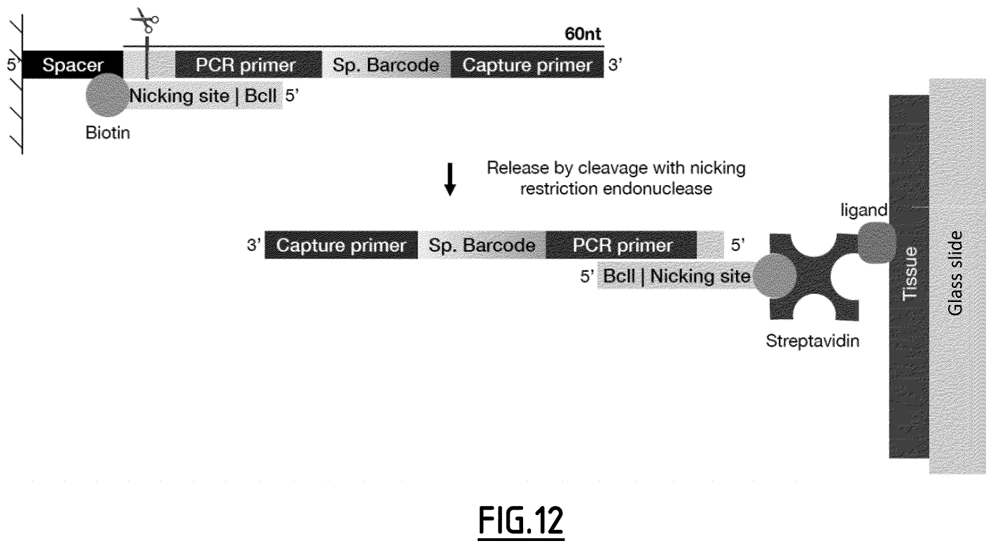

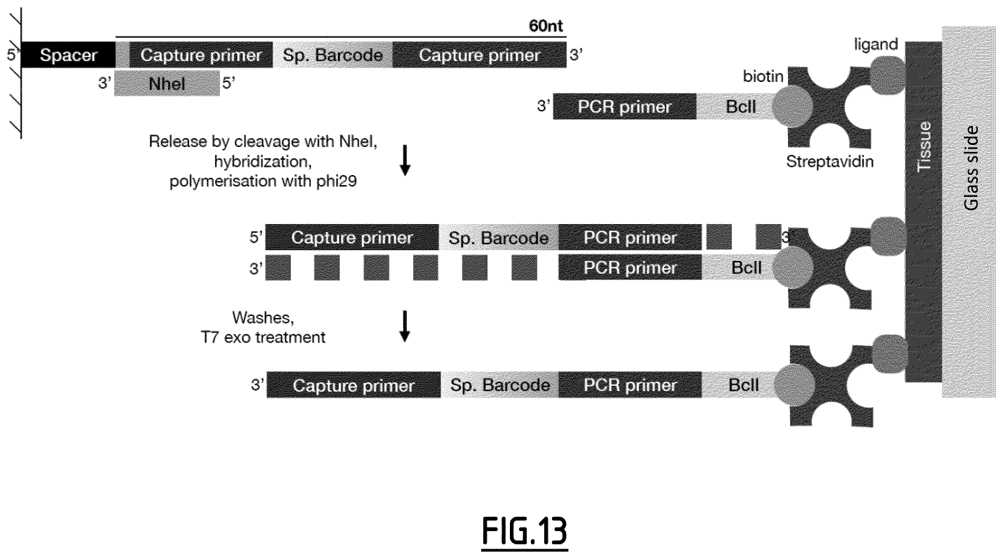

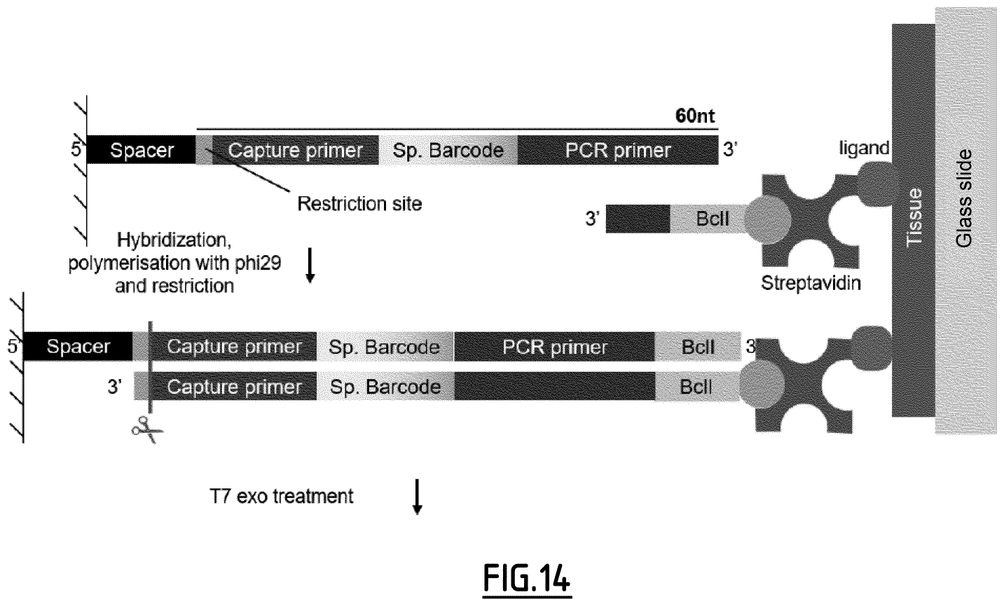

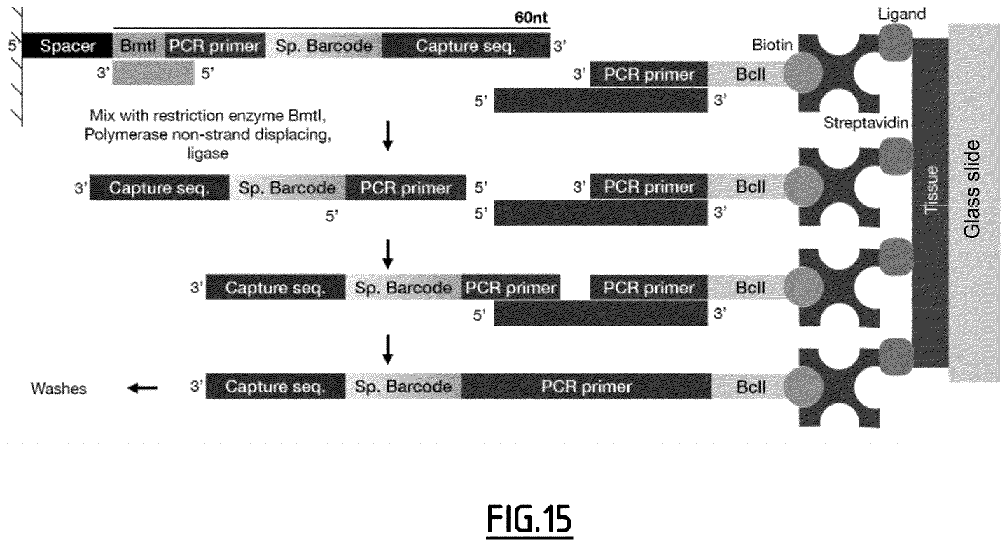

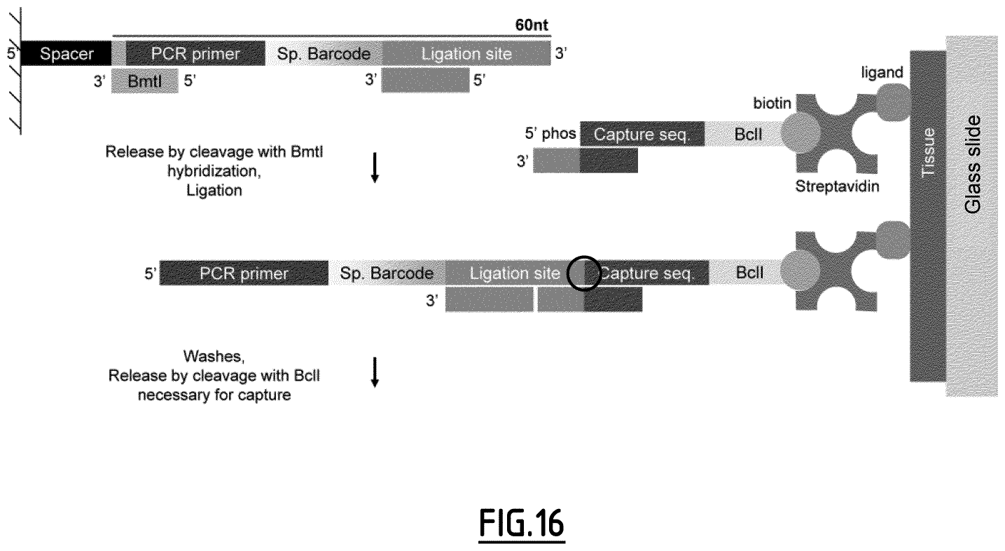

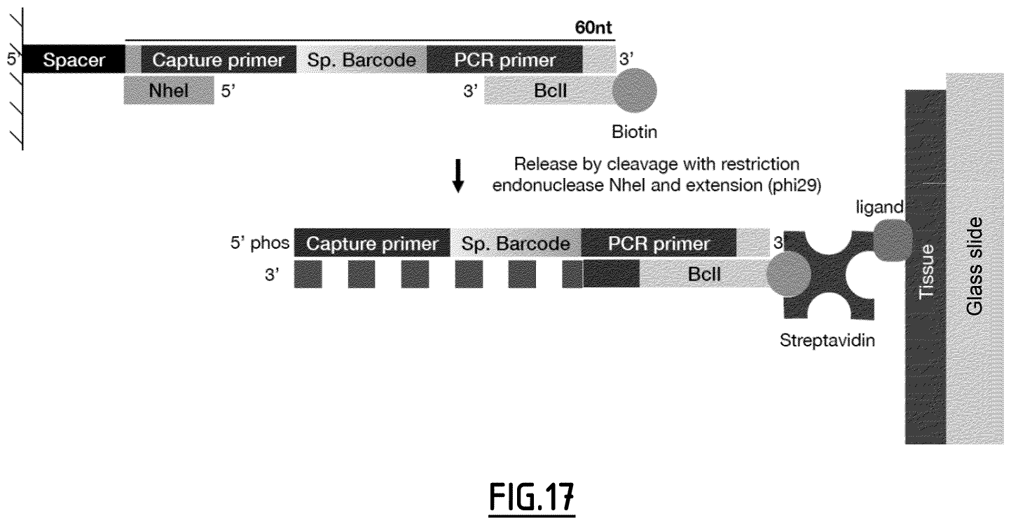

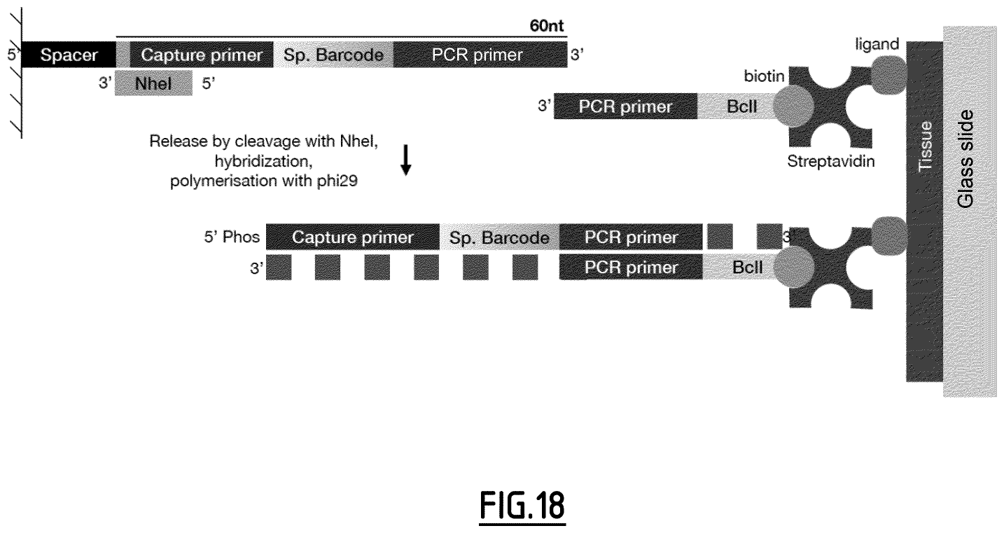

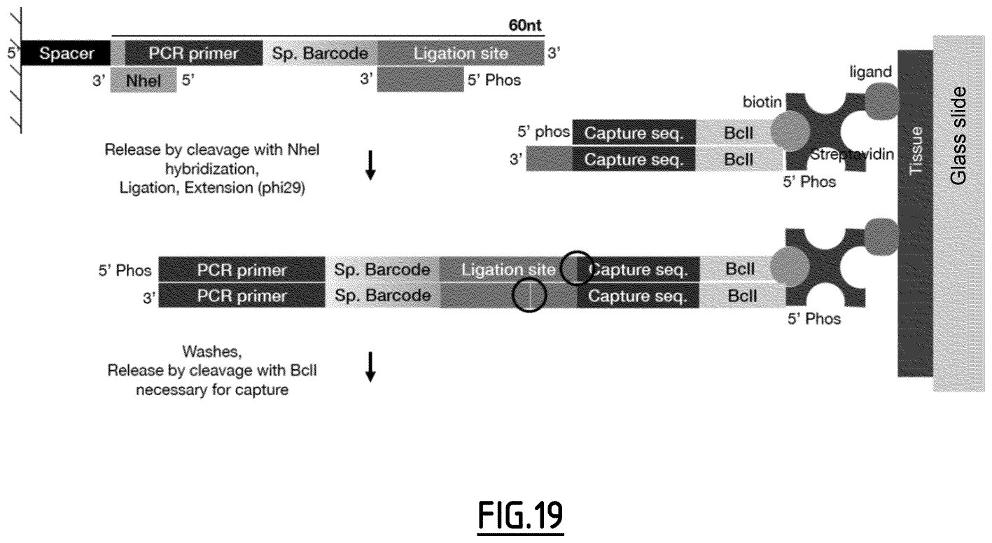

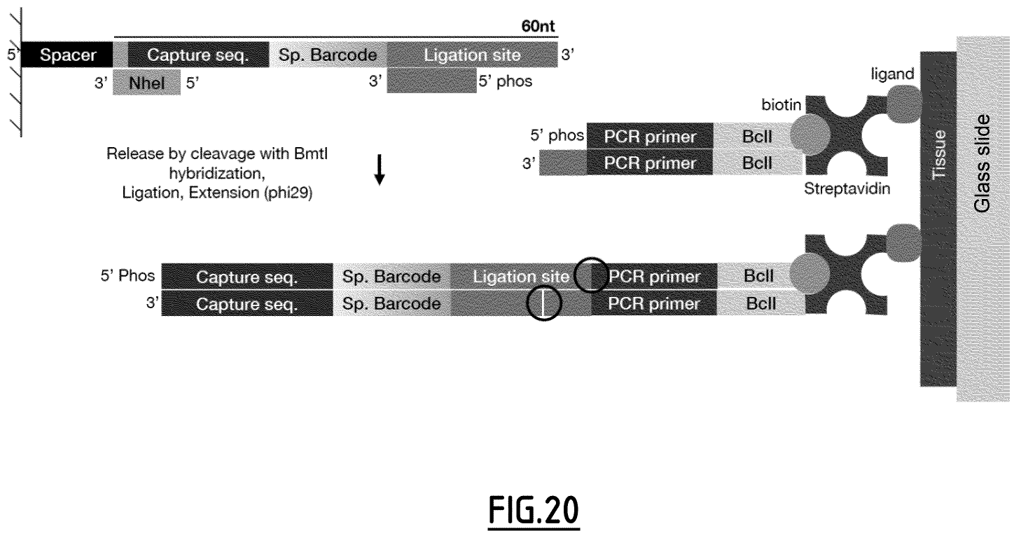

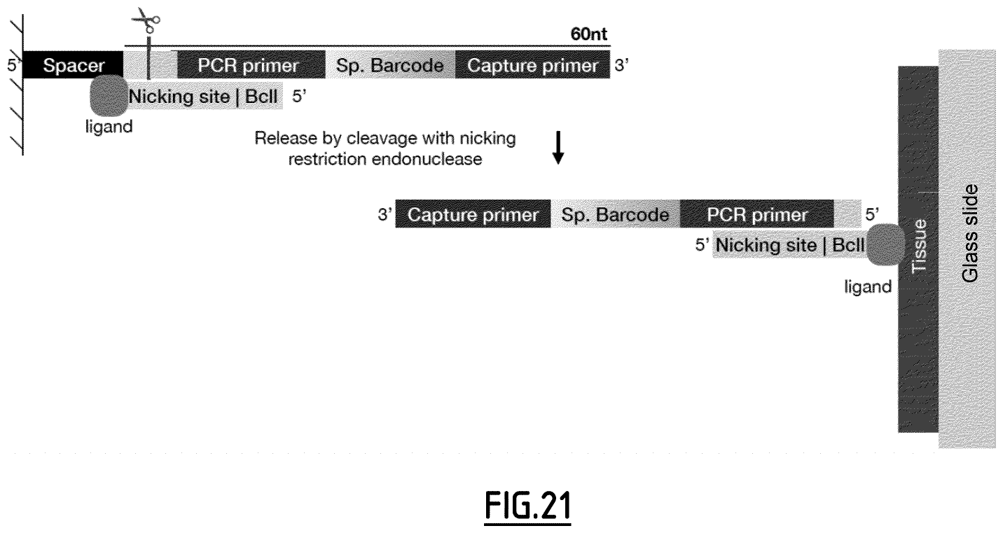

In examples 2 to 6, the molecular biology strategy used for cell labelling is according to the principles shown on FIG. 2.

In examples 7 to 8, the molecular biology strategy used for cell labelling is according to the principles shown on FIG. 2.

### Oligonucleotides

The oligonucleotides used in the example have the structures shown in Table 1.

TABLE 1
Oligonucleotide sequences

| Code | Name | Sequence (5′ to 3′) |
|---|---|---|
| A | SSP3c | CTGCGCGAACTGGCTAG |
| | | (SEQ ID NO: 7) |
| B | U2 | GATCACACTCGGCCATGCCGCC |
| | | (SEQ ID NO: 8) |
| C | BcII_U2c_ | ATGGCCGAGTGTGATCAGGT/3Bio/ |
| | 3Bio | (SEQ ID NO: 9) |
| D | Bmtl_SSP3 | TGCTAGCCAGTTCGC |
| | | (SEQ ID NO: 10) |
| E | refA_ | /5TYE563/AGCATGCGTCTATGC |
| | 5TYE563 | (SEQ ID NO: 14) |
| H | 5Bio_Ran2_ | /5Biosg/CAATCACTGACCGCCC |
| | BcII_U3 | TGAATGATCACTAGCGGCAGCCGC |
| | | GGATCGTT |
| | | (SEQ ID NO: 15) |
| I | Bmtl_U3_ | TCGATGCTAGCGGCAG/3ddC/ |
| | 3ddC | (SEQ ID NO: 16) |
| J | U3 | AACGATCCGCGGCTGCCGCTAG |
| | | (SEQ ID NO: 17) |

### DNA Microarray Structure

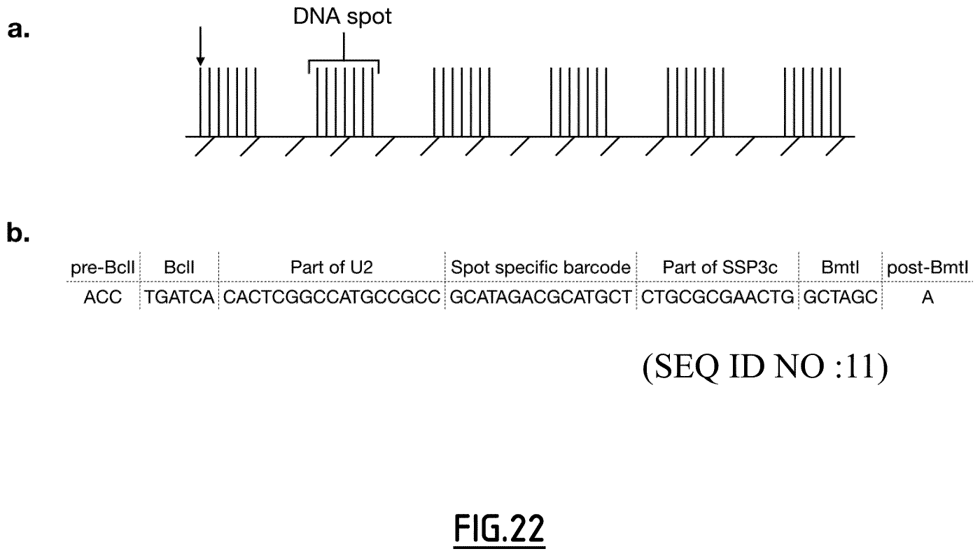

The DNA microarray general structure comprises an array substrate harboring ‘localisation’ nucleic acids forming spots, separated by inter-spot spaces where no nucleic acid is attached to the substrate. Nucleic acids from different spots carry different barcode sequences. The general structure of DNA micro-array and of an exemplary ‘localisation’ nucleic acid is shown in FIG. 22.

### Tissue Preparation

Fresh human biopsy of 20 to 200 μm thick, 8 mm diameter, adapted to clinical preparations.

### Example 2: Fresh Tissue Stamping and Dissociation

Anti-human β2-microglobulin Antibody (clone 2M2, AB_492835) is conjugated with streptavidin (ab102921).

Tissue slide is washed in a cell staining buffer (Biolegend 420201) before incubation with a mix of streptavidin conjugated and FITC (Biolegend 316304) anti-human β2-microglobulin antibodies at 5 μg/ml for 1 h at room temperature. The slide is then washed with TBS 1× (Sigma T5912). One bright field and two fluorescence (Absorbance Max: 495 nm, Emission Max: 521 nm and Absorbance Max: 549 nm, Emission Max: 563 nm) images can be taken at this step as a control for the staining and for oligonucleotide transfer, as well as for future alignment with the sequencing data.

In parallel, oligonucleotides C, D and E are hybridized to a DNA array (Agilent G4860A) comprising sequences as described in FIG. 22. A solution of oligonucleotides C, D and E at 20 μM in hybridization buffer (e.g. 100 mM Potassium Acetate; 30 mM HEPES, pH 7.5) is spread on the surface of the DNA array. In saturated humidity conditions, the slide is heated to 94° C. for 2 minutes and gradually cooled before being rinsed in TBS.

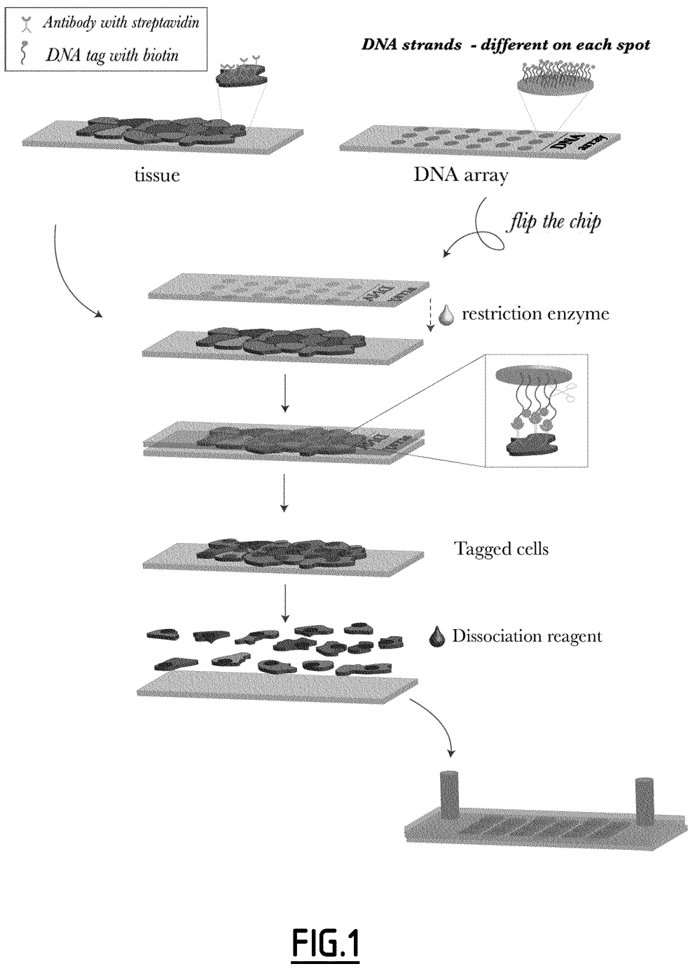

At 4° C., a solution containing Bmtl enzyme (NEB R3658) in 1× CutSmart buffer is spread at the surface of the DNA array and any liquid excess is discarded before contacting the previously prepared biopsy on the same surface. Without moving one part relative to the other, the DNA array, the enzymatic solution and the tissue are brought to 37° C. for 10 min before removing the tissue for dissociation (procedure presented in FIG. 1).

One bright field and two fluorescence (Absorbance Max: 495 nm, Emission Max: 521 nm and Absorbance Max: 549 nm, Emission Max: 563 nm) images can be taken at this step as a control tissue status, staining and level of oligonucleotide transfer, and for future alignment with the sequencing data.

After washing in HBSS (Thermo 14175053)+5% FBS (Thermo 16140071), the biopsy is minced as small as possible (around 1 mm).

The sample is then transfer to a tube with 200 μL of 20 mg/mL collagenase I (final: 2 mg/mL, Sigma C0130), 5 μL of 10 mg/mL Dnase I (final: 25 μg/mL, Sigma 11284932001), 80 μL of 50 mg/mL hyaluronidase (final: 2 mg/mL, Sigma H3506). The final volume is adjusted to 2 mL with HBSS. After 50 minutes of smooth agitation at 37° C. with regular up and down pipetting, the digested sample is filtered with a cell strainer (40 μm) and washed using TBS 1× (Thermo 14190169) with 1% HS (Thermo 26050088) and 2 mM EDTA (Thermo 15575020). Cells are resuspended in TBS 1×.

### Example 3: Fresh Tissue Stamping with a Lyophilized Stamp Followed by Dissociation

First steps of preparation of the DNA array are detailed in example 2.

At 4° C., a solution containing a custom glycerol free Bmtl enzyme (NEB R3658 type), 1-10% w/v of trehalose (Merck, USA), (6 mM MgCl2 and 2 mM dNTPs) is spread at the surface of the DNA array. The conditions of spin-coating are fixed at an angular velocity varying between 500 rpm and 3000 rpm for a spinning time of 30 s and kept at 4° C. Lyophilization is performed for 4 h in Modulyo Freeze Dryers (Thermo Electron Corporation, USA). The resulting DNA arrays are kept in a dry storage compartment.

Tissue sample is wetted in TBS 1× before being deposited on the lyophilized DNA array without moving one part relative to the other and brought to 37° C. for 10 min before removing the tissue for dissociation.

We then proceed with the same steps detailed in example 2.

### Example 4: Spatially Resolved scRNA-seq on Drop-seq Platform

In order to get a spatially resolved scRNA-seq, Drop-seq is performed as described in (Macosko, E. Z. et al. Cell 161, 1202-1214 (2015)) with modifications using the labeled cell from example 1. 10% of the oligonucleotide on the hydrogel beads are carrying at their 3′ end the reverse complementary of sequence A for cell position tag capture. Cell position tags are thus associated with the same droplet barcode as the RNA. Bcll endonuclease can be added to the emulsion mix to release the localization sequence from the cell.

cDNAs are separated and amplified as described in Stoeckius, M et al. Simultaneous epitope and transcriptome measurement in single cells. Nat Methods 14, 865-868 (2017) with modifications. Barcoded cell position tags are amplified using supplementary primers with sequence B at their 3′end.

After sequencing, the cell position can be identified through the cell position tags and link to mRNA transcript of the same cell thanks to the droplet barcode.

### Example 5: Spatially Resolved scRNA-seq in Cages

In order to get a spatially resolved scRNA-seq through the use thermo-actuable cages, single-cell isolation, barcoding and sequencing is performed as described in patent BV19034 with modifications using the labeled cell from example 1. 10% of the oligonucleotide on each spot are carrying at their 3′ end the reverse complementary of sequence A for cell position tag capture. Those oligonucleotides for cell position tag capture are hybridized along with RNA capture sequence during chip preparation.

After cell lysis, cell position tags are thus associated with the same droplet barcode as the RNA.

After collection of the cDNAs, an amplification is performed using supplementary primers with sequence B at their 3′end to amplify barcoded cell position tags.

After sequencing, the cell position can be identified through the cell position tags and link to mRNA transcript of the same cell thanks to the droplet barcode.

### Example 6: Spatially Resolved scCHIP-seq

Anti-human β2-microglobulin Antibody (clone 2M2, AB_492835) is conjugated with streptavidin (ab102921).

Oligonucleotides H is mixed with the streptavidin conjugated antibody in a 12:1 molar ratio.

Tissue slide is washed in a cell staining buffer (Biolegend 420201) before incubation with a mix of Oligonucleotides H with streptavidin conjugated and FITC (Biolegend 316304) anti-human β2-microglobulin antibodies at 5 μg/ml for 1 h at room temperature. The slide is then washed with TBS 1× (Sigma T5912). One bright field and two fluorescence (Absorbance Max: 495 nm, Emission Max: 521 nm and Absorbance Max: 549 nm, Emission Max: 563 nm) images can be taken at this step as a control for the staining and for oligonucleotide transfer, as well as for future alignment with the sequencing data.

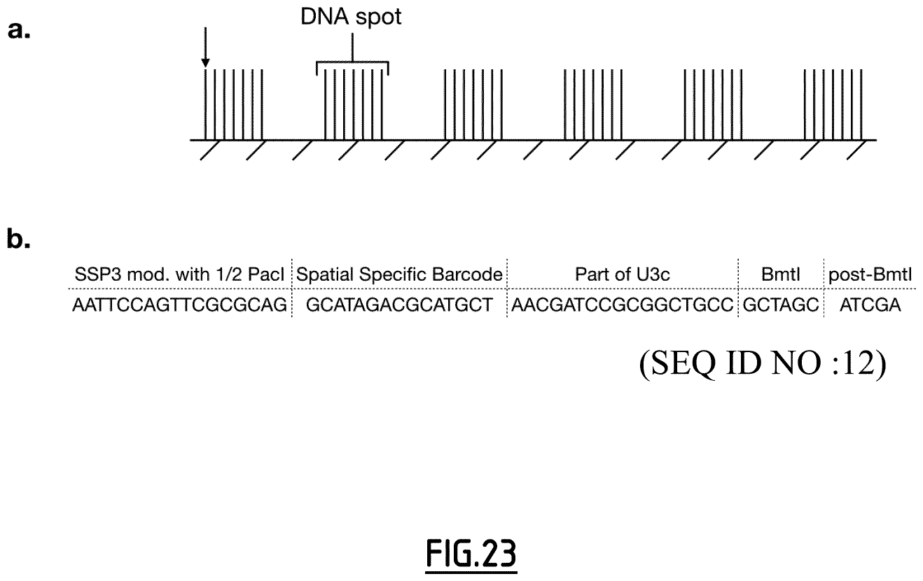

A solution containing the enzyme T4 Polynucleotide Kinase (NEB M0201) in 1× T4 Polynucleotide Kinase Reaction Buffer is spread on a DNA array (Agilent G4860A), comprising sequences as described in FIG. 23, and incubated for 30 min at 37° C. to proceed to 5′ phosphorylation of the strands anchored on the DNA array.

Oligonucleotides I and E are then hybridized to the DNA array. A solution of oligonucleotides I and E, at 20 μM in hybridization buffer (e.g. 100 mM Potassium Acetate; 30 mM HEPES, pH 7.5) is spread on the surface of the DNA array. In saturated humidity conditions, the slide is heated to 94° C. for 2 minutes and gradually cooled before being rinsed in TBS.

At 4° C., a solution containing Bmtl enzyme (NEB R3658) and phi29 (NEB. M0269) in 1× CutSmart buffer is spread at the surface of the DNA array and any liquid excess is discarded before contacting the previously prepared biopsy on the same surface. Without moving one part relative to the other, the DNA array, the enzymatic solution and the tissue are brought to 37° C. for 10 min before removing the tissue for dissociation (procedure presented in FIG. 1).

The following steps for imaging and dissociation are described in example 2.

After dissociation the labeled cells are used for the scChIP-seq procedure as described in Grosselin, K., Durand, A., Marsolier, J. et al. High-throughput single-cell ChIP-seq identifies heterogeneity of chromatin states in breast cancer. Nat Genet 51, 1060-1066 (2019).

After collection of the dsDNA, an amplification is performed using supplementary primers with sequence J at their 3′end to amplify barcoded cell position tags.

### Example 7: Spatially Resolved scATAC-seq in Cages

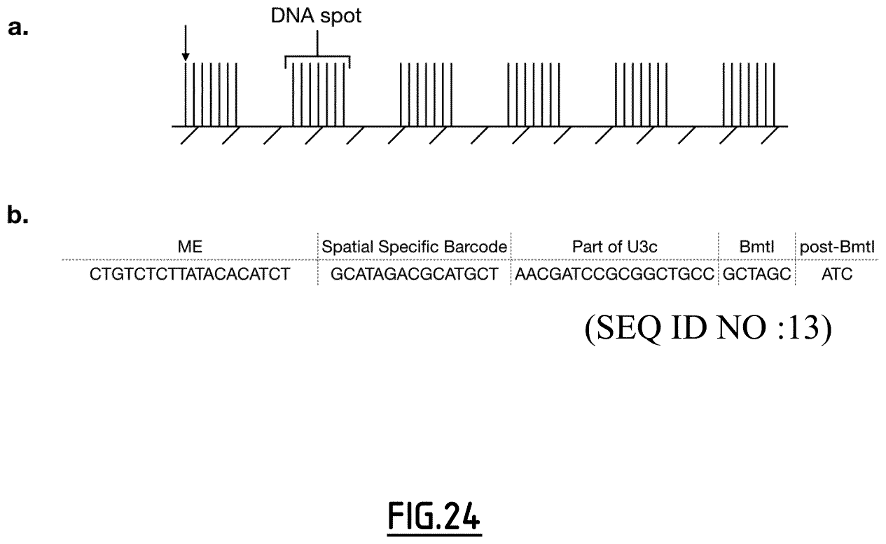

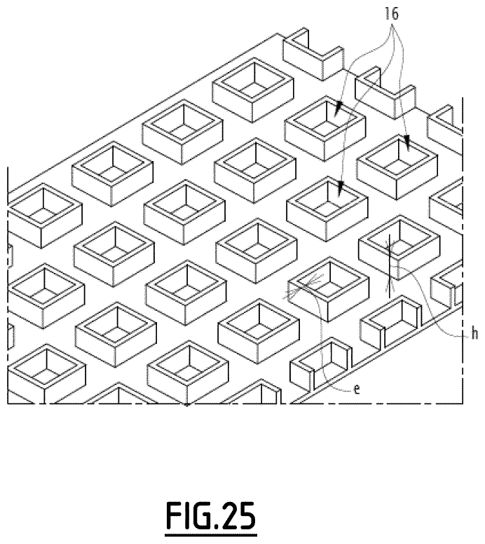

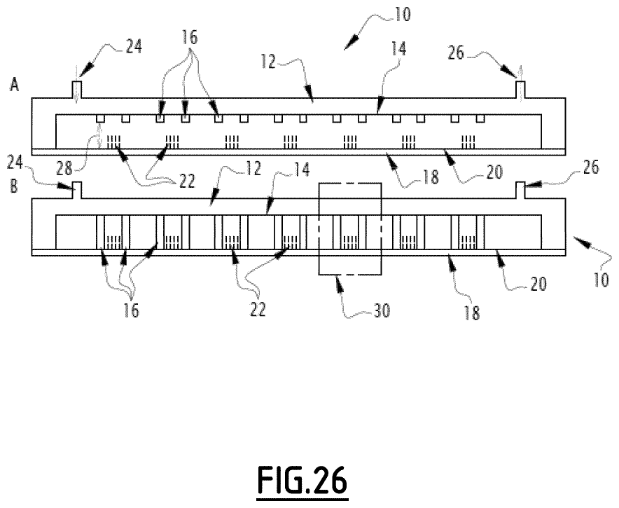

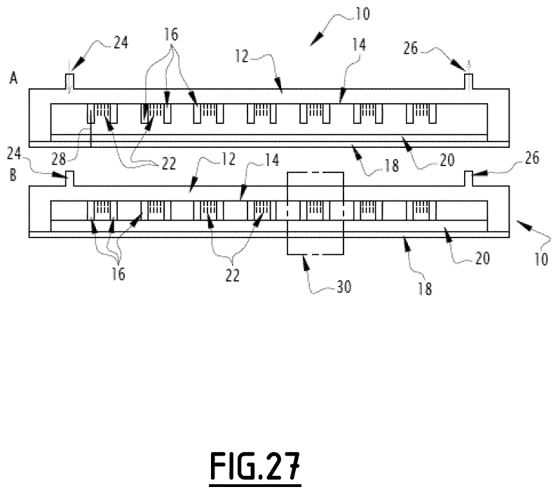

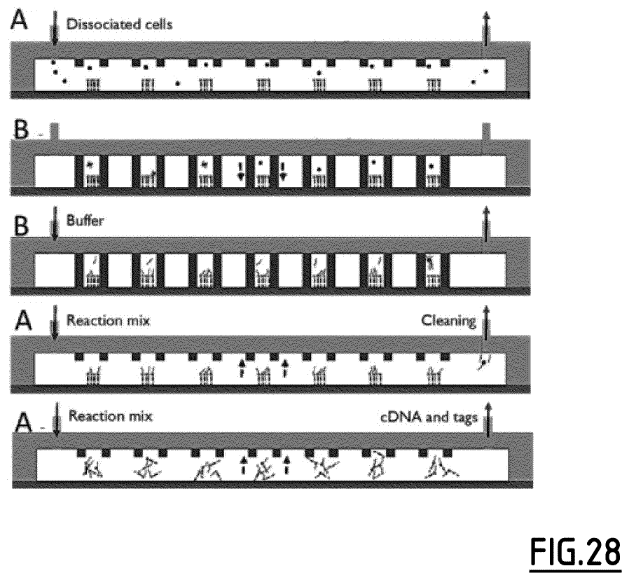

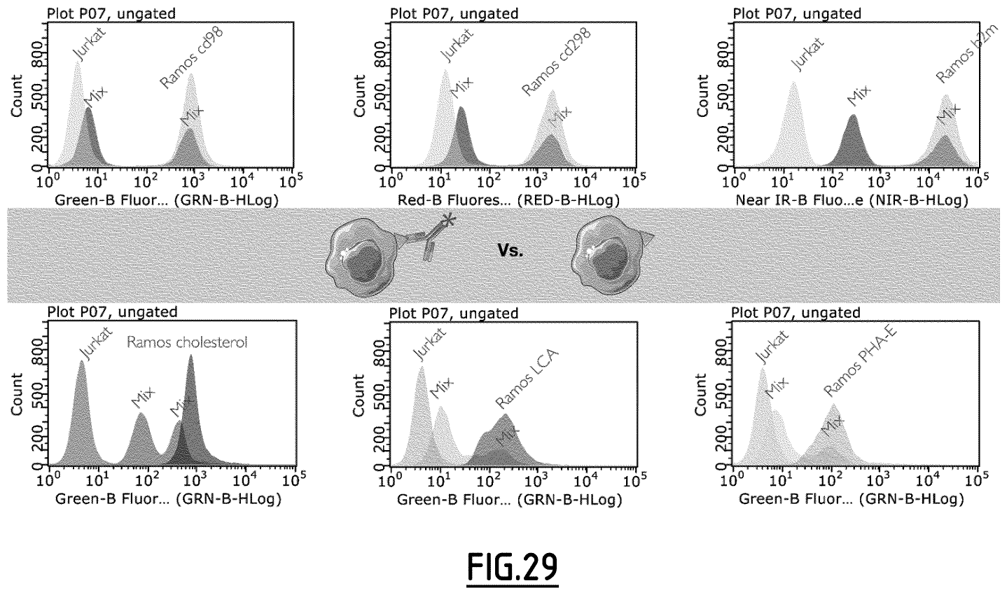

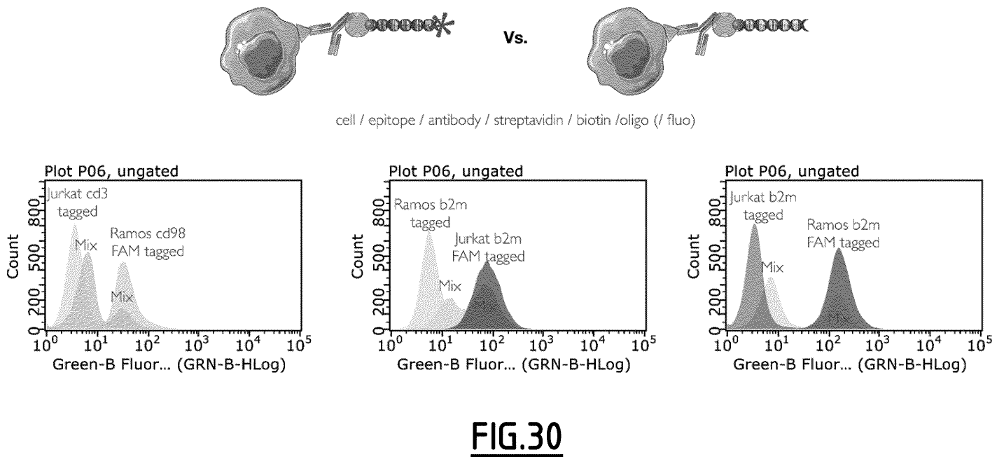

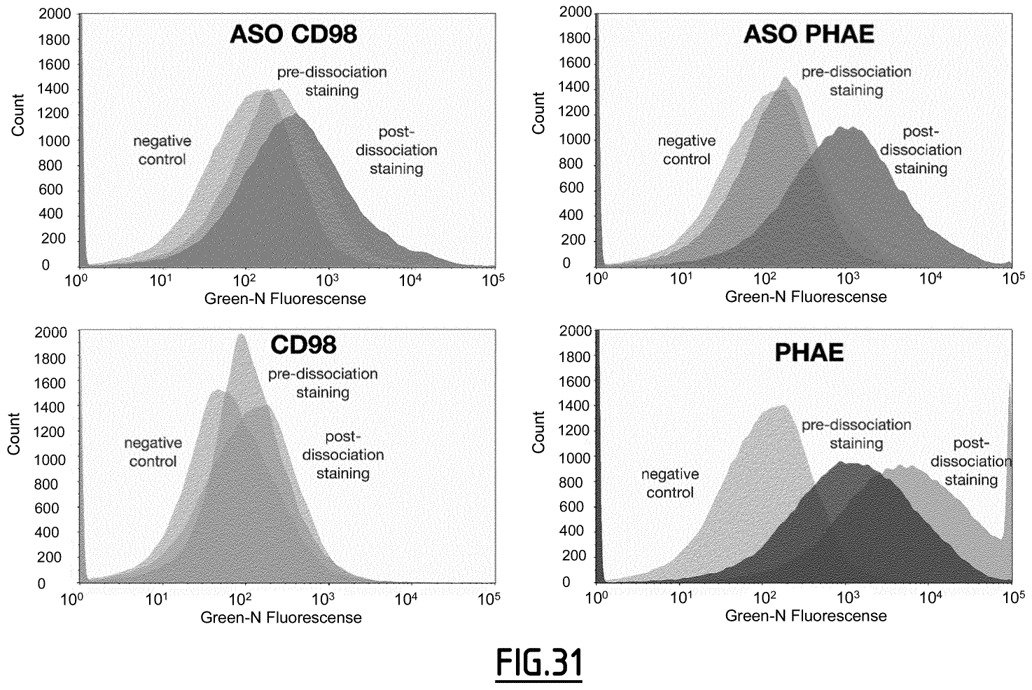

In order to get a spatially resolved scATAC-seq through the use thermo-actuable cages, single-cell isolation, barcoding and sequencing is performed as described FIG. 28 using labeled cells. Cells are labeled according to example 6 using a DNA array comprising sequences as presented in FIG. 24.

### Synthesis of the Thermos-Actuatable Hydrogel Showing UCST Behavior

A ene-functionalized UCST polymer is synthesized by free radical polymerization of methacrylamide (MA), acrylamide (AM) and allylmethacrylate (AMA) with 90:5:5 molar ratio using 2,2′-azobis(2-methylpropionamidine)dihydrochloride (V50) as thermal radical initiator. MA (8 g, 94 mmol), AM (0.371 g, 5.2 mmol), AMA (0.659 g, 5.2 mmol) and V50 (0.071 g, 0.3 mmol) were mixed in 247 mL of water and 123 mL of formamide. The solution was deoxygenated by nitrogen bubbling under the reflux condition at 55° C. for 1 h. The medium was allowed to proceed under refluxed condition at 55° C. under nitrogen for 24 h. The polymer solution was then dialyzed in pure water for four days at 70° C. The ene-functionalized UCST P(MA-AM-AMA) terpolymer was finally recovered by freeze-drying.

The swelling properties of the obtained polymer, as a function of temperature, are evaluated in a phosphate saline buffer.

### Preparation of the First Substrate

The first substrate, made of polydimethylsiloxane (PDMS), is prepared by standard soft lithography techniques, containing microstructures and a chamber. The height of the structures and the chamber depends on the objective and can be a couple of a tenth of a micron up to 100 microns tall.

### Functionalization of the First Substrate

The PDMS substrate is exposed to Oxygen plasma for 50s after being cleaned with isopropanol. Immediately following the surface activation, a solution of anhydrous toluene with a 3 vol % of mercaptopropyltrimethoxysilane (ABCR Gelest) is put in contact with the substrate for 3 h inside a reactor under nitrogen. Following the thiol-modification of the surface, the substrate is rinsed with toluene and finally dried with nitrogen flow.

### Photo-Patterning of Hydrogel Films onto the First Substrate

P(MA-AM-AMA) terpolymer (ene-reactive UCST polymer) is spin-coated on the thiol-modified & micro-structured PDMS substrate with dithiol cross-linkers at a temperature of 40° C. at least. A microvolume of a couple of 100 uL of a solution of acetic acid (V/V=1/1) containing P(MA-AM-AMA) polymer at a concentration between 3 and 15% wt and dithioerythritol (purchased from Sigma Aldrich, CAS number 3483-12-3) cross-linkers at a concentration between 3 and 10% wt is deposited onto the substrate. The conditions of spin-coating are fixed at an angular velocity varying between 500 rpm and 3000 rpm for a spinning time of 30 s. The spread films are dried by heat in a 90° C. oven for 5 minutes, in a water saturated environment. The resulting layer thickness varies from a few tenth of a micron to 15 microns.

Chromium masks presenting numerous microstructured cages are aligned with the design of the chamber and placed under UV lamp for deep UV exposure (8 watts, 250 nm wavelength). After exposure, free polymer chains are rinsed off by washing the substrate in an ultrapure water bath for 5 minutes. The hydrogel-patterned substrates are dried with nitrogen flow.

### Preparation of the Second Substrate

The second substrate used is a glass slide spotted with DNA strands (purchased from Agilent, referenced as an Agilent Microarray Format).

Presenting up to 1 million unique spots with different DNA strands grafted on it, this item provides different barcode on each spot. Each spot containing millions of DNA strands.

The micro array structure of the DNA is represented on FIGS. 22-24, each spot having a different barcode.

A localization system is integrated in the design of the array. Among the numerous unique spots, some of them carry a specific sequence for fluorescent labels capture (2 or more). They are placed in a way they form multiple shapes comprising triangle, square and circle.

### Closure of the Device

Bonding between the first and the second substrates is achieved by using an O2 plasma treatment for 50 s. A protective layer is tapped onto the area of interest avoiding Oxygen plasma to be active there. After the termination of the exposure, the PDMS substrate is placed on top of the DNA array so that the region of interest faces the hydrogel structures. A curing step is applied, for 30 minutes at least, by storing the chip inside a 70° C. oven.

### Preparation of the Chip for Specific Capture: scATAC-seq

A solution containing the enzyme T4 Polynucleotide Kinase (NEB M0201) in 1× T4 Polynucleotide Kinase Reaction Buffer is injected inside the microfluidic chamber at 20° C. (cages open) and incubated for 30 min at 37° C. to proceed to 5′ phosphorylation of the strands anchored on the DNA array.

The chamber is then rinsed with PBS solution at 20° C.

A solution containing the enzyme phi29 in 1× phi29 DNA Polymerase Reaction Buffer with 20 μm of oligonucleotide complementary to the 3′ end of DNA array nucleic acids is injected inside the microfluidic chamber at 20° C. (cages open) to create a double stranded DNA adaptor (barcoded MEDS).

After 10 min incubation at 37° C., the chamber is rinsed with PBS solution at 20° C.

### Capture, Lysis and Library Preparation

A suspension of cells at a concentration of 10 million per ml with 1% Pluronic f68, 15% Optiprep and 1% BSA in TBS is prepared. Cells are injected inside the chip at 100 μl/h and at 20° C. Once the cells are circulating around and above the cages, the flow is stopped. The cages are closed by heating the chip higher than 37° C.

To perform the lysis, a solution of low salted water is injected inside the chamber through additional inlets that are not obstructed by the swollen hydrogel cages.

The cleaning step is performed in this case at room temperature.

A mix containing enzyme Bmtl in CutSmart buffer 1× is injected at 37° C. and then stopped inside the chamber and incubated for 10 min at 37° C. to release the dsDNA from the DNA array.

A mix including Nextera Tn5 Transposase (TDE1) in 1× TD reaction buffer is then injected inside the chamber at 37° C., followed by an immobilization of the flow for 30 min at 37° C. to proceed to transposition.

Cell position tags are thus associated with the same cage barcode as the dsDNA.

The inner volume is then collected by flowing a solution of water at 20° C.

After an Exol treatment, a PCR amplification is performed on the collected sample.

The PCR product is then purified and quantified before being sequenced.

After collection of the dsDNA, an amplification is performed using supplementary primers with sequence J at their 3′end to amplify barcoded cell position tags.

After sequencing, the cell position can be identified through the cell position tags and link to dsDNA transcript of the same cell thanks to the cage barcode.

Reference: Buenrostro, J., Wu, B., Litzenburger, U. et al. Single-cell chromatin accessibility reveals principles of regulatory variation. Nature 523, 486-490 (2015)

### Example 8

To achieve a proof of concept applicable to a cohort of biological samples, we demonstrated that we could preserve the coupling of a DNA nucleic acids with cells, using antibodies, lectins or cholesterol-teg as ligand, from a tissue section during dissociation independently of the cell type.

### Cell Culture

Jurkat human T lymphocyte ATCC® TIB-152 and Ramos human B lymphocyte ATCC® CRL-1923 are cultivated in RPMI 1640 Medium (Gibco 61870044) supplemented with 10% heat inactivated Fetal Bovine Serum (Gibco 10082147) and 1% Penicillin-Streptomycin (Gibco 15140122). Cells are seeded on 25 cm2 or 75 cm2 culture flask at 37° C. with 5% CO2 following ATCC recommendations. On reaching 75-80% confluence the cells are diluted. After retrieving from cell culture, the cells are finally re-suspended in TBS 1× at the concentration of 2.10⁶ cells·mL⁻¹.

### Antibody and Lectin Conjugation

Purified antibody (Biolegend) and lectin (Eurobio Scientific) are firstly conjugated with streptavidin using Streptavidin Conjugation Kit Protocol (ab102921). Conjugated markers are then mixed in 1× tris buffered saline (TBS, VWR CAYM600232-500) with biotinylated oligonucleotides in 1:12 ratio and stored protected from light in a room with controlled temperature between 20 and 25° C. for over 12 h (over-night). Biotinylated oligonucleotides are purchased from IDT, in a 100 μM concentration in IDTE Buffer, pH 8.0, with standard desalting. The sequence of the biotinylated fluorescent oligonucleotide is: /56-FAM/CACAGGGTGATCAGGT/3Bio/. 56-FAM stands for a fluorescein fluorescent dye attached at the 5′ end of the oligo, 3Bio for a biotin attached at the 3′ end of the oligo.

### Tissue Dissociation

About 2 g of fresh colon sample were cut in pieces of about a mm². Before proceeding to staining, pieces of tissue were washed three times in 10 ml of 1× phosphate buffered saline (PBS, Gibco 10010023) followed by three washes in Cell Staining Buffer (Biolegend 420201) with DSS (Sigma D8906) at 400 μg/ml and 5 mM EDTA (Sigma 03690). Tissue was stained with 1 to 10 μg of antibodies or lectins conjugated with a fluorophore or a fluorescent oligonucleotide, in 500 μl of Cell Staining Buffer (Biolegend 420201) for 30 min at 4° C. Pieces were then washed in 10 ml of 1× phosphate buffered saline before proceeding to dissociation using gentleMACS Octo Dissociator and Tumor Dissociation Kit (Miltenyi Biotec 130-095-929). After dissociation cells were filtered at 40 μm, washed 10 ml of tris buffered saline (TBS, VWR CAYM600232-500) and resuspended in 1 ml TBS. Optionally, cells were stained with DAPI to distinguish living and dead cells.

### Cell Staining

200,000 cells are resuspended in 100 μL of Cell Staining Buffer (Biolegend 420201) with DSS (Sigma D8906) at 400 μg/ml and 5 mM EDTA (Sigma 03690). Cells were incubated with 5 μL of Fc Receptor Blocking Solution (Biolegend 422301) in the dark at 4° C. for 10 min followed by the addition of 0.2 to 2 μg of antibodies or lectins conjugated with a fluorophore or fluorescent oligonucleotides or equivalent quantity of cholesterol modified oligonucleotides. Cells were incubated for 30 min in the dark at 4° C. before being rinse twice in the Cell Staining Buffer mix previously described and twice in Tris buffered saline (TBS, VWR CAYM600232-500). For each wash, cells were centrifugated for 5′ at 130 rcf and 4° C., supernatant was removed except 50 μl before 200 μl of clean buffer was added. After the last wash, cells were resuspended in 200 μl TBS.

Cell staining is usually performed after tissue dissociation, however in order to labeled cells according to their initial position in the tissue, the staining needs to be done prior to dissociation.

We selected universal external cell markers in order to label every cell of the tissue, without need of permeabilization.

We firstly demonstrated using flow cytometry (Guava easyCyte 12HT) markers un-specificity and absence of marker exchange after staining on Jurkat and Ramos cell lines. Universal cell markers were chosen among anti-human CD98 (BioLegend 315603, 315602), anti-human CD298 (BioLegend 341709) or anti-human β2-microglobulin (BioLegend 316317, 316302), lectin jacalin, lectin LCA, lectin PHA-E and a cholesterol modification (3CholTeg at IDT with HPLC purification) at the 3′ end of the oligo in place of the biotin modification.

We stained each population with one of the markers before mixing a part of each stained population together for 30 min and analyzing them through flow cytometry. It appears that we were still able to distinguish each population after mixing, whatever the type of labeling (FIG. 29), indicating an absence of cross-contamination after staining. When using the oligo with the cholesterol modification, the separation is less important than for the antibodies or lectins but still present,

We also demonstrated that antibody conjugated oligonucleotide do not exchange their oligonucleotide via the biotin streptavidin linkage by mixing a population stained with markers conjugated with fluorescent oligonucleotides and a population stained with markers conjugated with non-fluorescent oligonucleotides (FIG. 30).

Finally, we demonstrated the resistance of selected cell markers to tissue dissociation. Each time, we compared unstained tissue with tissue stained before and after dissociation, and we assessed the presence of the staining by flow cytometry (Guava easyCyte 12HT).

With antibodies or lectins, using a fluorophore or a conjugation with a fluorescent oligonucleotide, the labeling was partially kept during the tissue dissociation (FIG. 31). The difference of staining between the pre and post dissociation conditions may be explained by the inability of markers to fully stain the inner part of tissue blocks.

In parallel, we confirmed that we could recover both transcriptomic and labeling nucleic acid information associated with the same cell, with a method similar to Cite-seq method (Stoeckius et al., « Simultaneous Epitope and Transcriptome Measurement in Single Cells ».).

1. A method for labelling individual cells or organelles within a tissue sample with a localisation nucleic acid, the method comprising:
a) providing a nucleic acid array comprising a substrate and localisation nucleic acids, wherein each localisation nucleic acid comprises, from the 5′ end to the 3′ end:
i. a first constant sequence;
ii. a localisation sequence that is indicative of the position of the nucleic acid on the nucleic acid array; and
iii. a second constant sequence;
wherein the localisation nucleic acid is attached, covalently or non-covalently, to the substrate of the nucleic acid array;
b) contacting the nucleic acid array with a tissue sample so that the localisation nucleic acids are at the interface between the nucleic acid array and the tissue sample:
c) releasing all or part of the localisation nucleic acids comprising all or part of the first constant sequence, the localisation sequence, and all or part of the second constant sequence or a nucleic acid complementary thereto;
d) binding of the released nucleic acids comprising the localisation sequence or its complementary sequence, to cells or organelles of the tissue sample via a ligand bound to a receptor or receptors of cells or organelles in the tissue sample;
e) dissociating the tissue sample and recovering individualized cells or organelles, wherein at least a sub-population of the individualised cells or organelles is labelled with the nucleic acid comprising the localisation sequence or its complementary sequence.

2. A method for labelling individual cells or organelles within a tissue sample with a localisation nucleic acid, the method comprising:
a) providing a nucleic acid array comprising a substrate and localisation nucleic acids, wherein each localisation nucleic acid comprises, from the 5′ end to the 3′ end:
iv. a first constant sequence;
v. a localisation sequence that is indicative of the position of the nucleic acid on the nucleic acid array; and
vi. a second constant sequence;
wherein the localisation nucleic acid is attached, covalently or non-covalently, to the substrate of the nucleic acid array;
b) contacting the nucleic acid array with a polymer stamp so that the localisation nucleic acids are at the interface between the nucleic acid array and the polymer stamp;
c) releasing all or part of the localisation nucleic acids comprising all or part of the first constant sequence, the localisation sequence, and all or part of the second constant sequence or a nucleic acid complementary thereto;
d) contacting the polymer stamp with a tissue sample:
e) binding of the released nucleic acids comprising the localisation sequence or its complementary sequence, to cells or organelles of the tissue sample via a ligand bound to a receptor or receptors of cells or organelles in the tissue sample;
f) dissociating the tissue sample and recovering individualized cells or organelles, wherein at least a sub-population of the individualised cells or organelles is labelled with the nucleic acid comprising the localisation sequence or its complementary sequence.

3. A method for labelling individual cells or organelles within a tissue sample with a localisation nucleic acid, the method comprising:
A) providing a nucleic acid array comprising a substrate and localisation nucleic acids, wherein each localisation nucleic acid comprises, from the 5′ end to the 3′ end:
i. a first (5′) constant sequence;
ii. a localisation sequence that is indicative of the position of the nucleic acid on the nucleic acid array; and
iii. a second (3′) constant sequence;
wherein the localisation nucleic acid is attached, covalently or non-covalently, to the substrate of the nucleic acid array;
B) providing a tissue sample labelled with a ligand which binds to a receptor or receptors of cells or organelles in the tissue sample, wherein the ligand is attached, covalently or non-covalently, to a capture nucleic acid which comprises at its 3′ end a sequence complementary to all or part of said second (3′) constant sequence;
C) contacting the nucleic acid array with the tissue sample so that the localisation nucleic acids are at the interface between the nucleic acid array and the tissue sample;
D) hybridising the localisation nucleic acid and capture nucleic acid, and extending the 3′ end of capture nucleic acid by polymerisation, thereby synthesising a sequence complementary to said localisation nucleic acid;
E) releasing all or part of the localisation nucleic acids comprising all or part of the first (5′) constant sequence, the localisation sequence, and all or part of the second (3′) constant sequence;
F) dissociating the tissue sample and recovering individualized cells or organelles, wherein at least a sub-population of the individualised cells or organelles is labelled with the capture nucleic acid comprising a sequence complementary to the localisation sequence.

4. The method according to claim 1, wherein the ligand binds to a receptor or receptors at the surface of or inside the cells, or the surface of or inside an organelle of the cells.

5. The method according to claim 1, wherein the 5′ end or the 3′ end of the localisation nucleic acid is attached, covalently or non-covalently, optionally via a spacer sequence and/or linker, to the substrate of the nucleic acid array.

6. The method according to claim 1, wherein the receptor or receptors at the surface or inside the cells or any organelles of the cells bound by the ligand is ubiquitously present at the surface or inside all or most of the cells or any organelles of the cells in the tissue sample.

7. The method according to claim 1, wherein the localisation nucleic acids form spots at the surface of the nucleic acid array, and all localisation nucleic acids of a same spot comprise a same localisation sequence which is specific for one or more spots.

8. A method of mapping and sequencing individual cells or organelles of a tissue sample, the method comprising:
a) providing individualized cells labelled with a nucleic acid comprising a localisation sequence as obtainable by the method of claim 1;
b) trapping the individualized cells or organelles labelled with the nucleic acid comprising the localisation sequence in a compartment, wherein the compartment comprises a compartment-specific nucleic acid and one or any combination of the following sequences for nucleic acid labeling and further sequencing: hybridization site, ligation site or recombination site;
c) optionally analysing captured cells, organelles and/or molecules they secrete, using optical detection;
d) optionally, lysing trapped cells, or cells and organelles, thereby releasing nucleic acids from the cells or organelles in the compartments;
e) associating the compartment-specific sequence with the nucleic acids released from the cells or organelles in the compartments and the nucleic acids comprising the localisation sequence;
f) recovering the nucleic acids produced at step e) from the compartments and sequencing recovered nucleic acids; and
g) defining nucleic acids comprising the same compartment-specific sequence as originating from the same single cell, and mapping the position of the single cell originally on the tissue sample based on the localisation sequence(s) contained in part of the nucleic acid that comprises said compartment-specific sequence, thereby combining mapping and sequencing information of the individual cells of the tissue sample;
h) optionally, mapping the sequencing information back onto an image from microscopy, optionally fluorescent microscopy, of the tissue, taken before dissociation.

9. The method of mapping and sequencing according to claim 8, wherein the compartments are droplets, microfabricated chambers separated by pneumatic valves, microfabricated wells, actuatable hydrogel cages or microplate wells.

10. The method of mapping and sequencing according to claim 1, wherein the compartment is a compartment of a microfluidic device comprising:
a first wall comprising a first substrate on which a plurality of closed patterns is grafted,
a second wall, facing the first wall, comprising a second substrate,
a plurality of nucleic acids grafted either on the first substrate or on the second substrate, each nucleic acid comprising a barcode that encodes the position of the nucleic acid on said first or second substrate,
wherein at least the plurality of closed patterns or the second substrate is made of an actuatable hydrogel which is swellable between a retracted state and a swollen state in which the closed patterns and the second substrate come into contact.

11. The method of mapping and sequencing according to claim 10, wherein a plurality of ligands is grafted on the first substrate and/or on the second substrate, or a plurality of ligands conjugated with a nucleic acid is associated by hybridization to at least part of the grafted nucleic acids.

12. The method of mapping and sequencing according to claim 10, wherein each ligand is independently chosen from the group consisting of antibodies, fragments of antibody, lectins, and aptamers.

13. The method of mapping and sequencing according to claim 1, which comprises:
a) providing the microfluidic device and a preparation of cells or organelles labelled with a nucleic acid comprising a localisation sequence as obtainable by the method of the invention;
b) optionally, associating all or part of the cells or organelles labelled with the nucleic acid comprising the localisation sequence in a compartment with a common labeling nucleic acid sequence or with a plurality of different labeling nucleic acid sequences;
c) injecting in the microfluidic device the cells or organelles labelled with the nucleic acid comprising the localisation sequence in suspension under conditions in which the hydrogel is in retracted state;
d) modifying the conditions to actuate the hydrogel into swollen state, thereby trapping cells or organelles in a cage formed by the first and second walls of the microfluidic device, and the closed pattern of hydrogel in swollen state;
e) optionally analyzing captured cells or organelles and/or molecules they secrete, using optical imaging;
f) optionally releasing in the cage, the grafted nucleic acids from the surface of the first or second substrate of the microfluidic device;
g) optionally, lysing trapped cells or organelles, thereby releasing cellular or organellar nucleic acids in the cages;
h) associating the barcode of the nucleic acids with either the released cellular or organellar nucleic acids and/or labeling nucleic acid sequence(s) thereby forming barcoded nucleic acids;
i) modifying the conditions to actuate the hydrogel into the retracted state;
j) releasing the grafted nucleic acids from the first or second substrate of the microfluidic device, if not released in f);
k) recovering and sequencing the barcoded nucleic acids; and
i) optionally mapping the barcoded sequencing data onto the data from optical imaging obtained in e).

14. The method of mapping and sequencing according to claim 13, wherein said method further comprises:
α) Binding of an analyte or of analytes secreted or released by the captured cells or organelles to ligands grafted directly or indirectly to the surface of the first substrate and/or on the surface of the second substrate;
β) Detecting the analyte or analytes bound to the grafted ligand by binding with a labeled second ligand or labeled ligands that is/are specific for the bound analyte or analytes.

15. The method of mapping and sequencing according to claim 14, wherein at step β), detecting is performed
i) directly with a second ligand or ligands fluorescently labeled; or
ii) indirectly, with a second ligand or ligands labeled with a ligand identification nucleic acid that is specific to the analyte or analytes that is(are) bound to the grafted ligand, wherein the sequence of said ligand identification nucleic acid allows identification of the ligand and the analyte or analytes bound to the grafted ligand and becomes associated with the barcode of the nucleic acid, thereby forming barcoded nucleic acids.

16. The method of mapping and sequencing according to claim 8, wherein single cells or single organelles are trapped in a compartment.

17. The method of mapping and sequencing according to claim 8, wherein the compartment-specific nucleic acid comprised in the compartment is DNA.

18. The method of mapping and sequencing according to claim 8, wherein step e) comprises:
i) hybridizing the compartment-specific nucleic acid by complementarity to the released nucleic acids from the cell or organelle;
ii) hybridizing the compartment-specific nucleic acid by complementarity to the released nucleic acids from the cell or organelle and extending the compartment-specific nucleic acid hybridized to the released nucleic acids using a DNA polymerase to create the complementary strand of the released nucleic acids with an associated compartment-specific sequence;
iii) hybridizing the compartment-specific nucleic acid by complementarity to the 3′-end of cDNA produced by reverse transcription of RNA from the cell or organelle and extending the cDNA hybridized to the compartment-specific nucleic acid using a DNA polymerase to create the complementary strand of the compartment-specific nucleic acid with an associated compartment-specific sequence;
iv) ligating the compartment-specific nucleic acid to the DNA present in the compartment; or
v) recombining the compartment-specific nucleic acid with the DNA present in the compartment.

19. The method of mapping and sequencing according to claim 8, wherein:
the compartment-specific nucleic acids comprising a primer sequence complementary to all or part of the second constant sequence present in the localisation nucleic acids
step e) additionally comprises hybridizing the compartment-specific nucleic acids to all or part of the second constant sequence present in the localisation nucleic acids and extending one or both hybridized DNA strands using a DNA polymerase to create a DNA molecule comprising both the localisation sequence, or its complement, and the compartment-specific sequence, or its complement.

20. A kit for labelling individual cells or organelles within a tissue sample with a localisation nucleic acid sequence, which comprises:
a) a nucleic acid array comprising nucleic acids, wherein each nucleic acid comprises, from the 5′ end to the 3′ end:
a first constant sequence;
a localisation sequence that is indicative of the position of the nucleic acid on the nucleic acid array; and
a second constant sequence;
wherein the nucleic acid is attached, covalently or non-covalently, to the substrate of the nucleic acid array;
b) at least one type of ligand that binds to a receptor of cells or organelles in the tissue, preferably a ubiquitously receptor present at the surface of the cells or organelles of a tissue sample, wherein the at least one type of ligand is optionally attached to a member of a non-covalent interacting pair; and
c) optionally both members of the non-covalent interacting pair.

21. A kit for mapping and sequencing individual cells or organelles of a tissue sample which comprises, as constituents of the kit:
a) the constituents of the kit according to claim 20; and,
b) trapping the individualized cells or organelles labelled with the nucleic acid comprising the localisation sequence in a compartment, wherein the compartment comprises a compartment-specific nucleic acid and one or any combination of the following sequences for nucleic acid labeling and further sequencing: hybridization site, ligation site or recombination site, wherein single cells or single organelles are trapped in the compartment.
